SEQUENCE LISTING (14 sequences)

SEQ ID NO: 1 - DNA, 2211 bp, adeno-associated virus rh.91
  CDS (1)..(2211)
atggctgccgatggttatcttccagattggctcgaggacaacctctctgagggcattcgcgagtggtgggcgctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaagcagctaagacggctcctggaaagaaacgtccggtagagcagtcgccccaagaaccagactcctcctcgggcattggcaaatcaggccagcagcccgccaaaaagagactcaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtacggctacctaacgctcaacaatggcagccaggccgtaggacgttcatccttttattgcctggaatatttcccatctcaaatgctgagaacgggcaacaactttaccttcagctacacctttgaagatgtgcctttccacagcagttacgcgcacagccagagcctggacaggctaatgaatcctctaatcgaccagtacctgtattacctaaacagaactcagaatcaatccggaagtgcacaaaacaaggacttgctgtttagccgggggtctccagctggcatgtctgttcagcccaaaaactggctacccgggccctgttaccgacagcagcgtgtttctaaaacaaaaacagacaacaacaacagcaactttacctggactggtgcctccaaatacaatctgaacggacgtgaatccatcattaaccctggcaccgctatggcatcccacaaggacgacgaagacaaattttttcccatgagcggtgttatgatttttggcaaagaaaatgcaggagcatcaaacactgcattagacaatgttatgattacagatgaagaggaaattaaagctaccaaccccgtggccaccgagagatttggaactgtggcagtcaatctccaaagcagcaatacagaccctgcaacaggagacgtgcatgtcatgggggctttacctggcatggtgtggcaagacagagacgtgtacctgcagggtcccatttgggccaagattcctcacacggatggacactttcacccgtctcctcttatgggcggctttggacttaagcacccgcctcctcagatcctcatcaaaaacacgcctgttcctgcgaatcctccggcagagttttcggctacaaagtttgcttcattcatcacccagtactccacaggacaagtgagcgtggaaattgaatgggagctgcagaaagaaaacagtaagcgctggaatcctgaagtgcagtacacctccaactacgcgaaatctgccaacgttgatttcactgtggacaacaatggactttatactgagcctcgccccattggcacccgttaccttacccgtcccctttaa

SEQ ID NO: 2 - protein, 736 aa, adeno-associated virus rh.91
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEAAKTAPGKKRPVEQSPQEPDSSSGIGKSGQQPAKKRLNFGQTGDSESVPDPQPLGEPPETPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKENAGASNTALDNVMITDEEEIKATNPVATERFGTVAVNLQSSNTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 3 - DNA, 2211 bp, Artificial Sequence
  synthetic construct
  CDS (1)..(2211)
atggctgctgacggttatcttccagattggctcgaggacaacctttctgaaggcattcgtgagtggtgggctctgaaacctggagcccctaaacccaaagcgaaccaacaaaagcaggacgacggccggggtcttgtgcttccgggttacaaatacctcggacccttcaacggactcgacaaaggagagccggtcaacgcggcggacgcggcagccctcgaacacgacaaagcttacgaccagcagctcaaggccggtgacaacccgtacctccggtacaaccacgccgacgccgagtttcaggagcgtcttcaagaagatacgtcttttgggggcaaccttggcagagcagtcttccaggccaaaaagagggttcttgagccttttggtctggttgaggaagcagctaaaacggctcctggaaagaagaggcctgtagagcagtctcctcaggaaccggactcatcatctggtattggcaaatcgggccagcagcctgccaaaaaaagactaaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtatggatacctcaccctgaacaacggaagtcaagcggtgggacgctcatccttttactgcctggagtacttcccttcgcagatgctaaggactggaaataacttcaccttcagctataccttcgaggatgtaccttttcacagcagctacgctcacagccagagtttggatcgcttgatgaatcctcttattgatcagtatctgtactacctgaacagaacgcaaaatcaatctggaagtgcacaaaacaaggacctgctttttagccgggggtctcctgctggcatgtctgttcagcccaaaaattggctacctgggccctgctaccggcaacagagagtttcaaagactaaaacagacaacaacaacagtaactttacctggacaggtgccagcaaatataatctcaatggccgcgaatcgatcattaatccaggaaccgctatggccagtcacaaggacgatgaagacaaatttttccctatgagcggcgttatgatatttggcaaagaaaatgcaggagcaagtaacactgcattagataatgtaatgattacggatgaagaagagattaaagctaccaatcctgtggcaacagagagatttggaactgtggcagtcaacttgcagagctcaaatacagaccccgcaactggagacgtccatgtcatgggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcctcatcaaaaatactccggtaccggcaaatcctccggcagagttcagcgctacaaagtttgcttcatttatcactcagtactccactggacaggtcagcgtggaaattgagtgggagctacagaaagaaaacagcaaacgttggaatccagaggtgcagtacacttccaactacgcgaagtctgccaatgtggactttactgtagacaacaatggtctttatactgaacctcgccctattggaacccggtatctcacacgacccttgtaa

SEQ ID NO: 4 - protein, 736 aa, Artificial Sequence; identical to SEQ ID NO: 2
  Synthetic Construct

SEQ ID NO: 5 - DNA, 2211 bp, Artificial Sequence
  AAV6 mutant
  CDS (1)..(2211)
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaacctcttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 6 - protein, 736 aa, Artificial Sequence
  Synthetic Construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - DNA, 2211 bp, adeno-associated virus 1
  CDS (1)..(2211)
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 8 - protein, 736 aa, adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 9 - DNA, 26 bp, Artificial Sequence
  primer sequence
gctgcgtcaactggaccaatgagaac

SEQ ID NO: 10 - DNA, 28 bp, Artificial Sequence
  primer sequence
cgcagagaccaaagttcaactgaaacga

SEQ ID NO: 11 - DNA, 25 bp, Artificial Sequence
  primer sequence
ggcgaacagcggacaccgatatgaa

SEQ ID NO: 12 - DNA, 25 bp, Artificial Sequence
  primer sequence
ggctctcgtcgcgtgagaatgagaa

SEQ ID NO: 13 - DNA, 22 bp, Artificial Sequence
  miRNA target sequence
agtgaattctaccagtgccata

SEQ ID NO: 14 - DNA, 24 bp, Artificial Sequence
  miRNA target sequence
agtgtgagttctaccattgccaaa

(19) United States
(12) Patent Application Publication
(10) Pub. No.: US 2023/0287451 A1
(43) Pub. Date: Sep. 14, 2023

(54) NOVEL AAV CAPSIDS AND COMPOSITIONS CONTAINING SAME

(71) Applicant: The Trustees of the University of Pennsylvania, Philadelphia, PA (US)

(72) Inventors: James M. Wilson, Philadelphia, PA (US); Kalyani Nambiar, New York, NY (US); Qiang Wang, Philadelphia, PA (US); Jenny Agnes Sidrane, Phoenixville, PA (US); Richard Alexander Martino, Philadelphia, PA (US)

(21) Appl. No.: 18/040,648

(22) PCT Filed: Aug. 13, 2021
(86) PCT No.: PCT/US21/45945

§ 371 Date: Feb. 5, 2023

Related U.S. Application Data
(60) Provisional application No. 63/109,734, filed on Nov. 4, 2020.
(60) Provisional application No. 63/065,616, filed on Aug. 14, 2020.

(51) Int. Cl. C12N 15/86; A61K 9/00

(52) U.S. Cl. CPC C12N 15/86; A61K 9/0019; C12N 2750/14122; C12N 2750/14143; C12N 2750/14152; C12N 2750/14145; C12N 2750/14171; A61K 48/00

(57) ABSTRACT

Provided herein are novel AAV capsids and recombinant AAV vectors comprising the same. In one embodiment, vectors employing a novel AAV capsid show increased transduction of a selected target tissue as compared to a prior art AAV.

### BACKGROUND OF THE INVENTION

Adeno-associated viral (AAV) vectors are safe and effective gene transfer vehicles used for several clinical indications. Treatment approaches based on AAV vectors have been approved by the US Food and Drug Administration and other worldwide regulatory authorities for the treatment of Leber congenital amaurosis, lipoprotein lipase deficiency, and spinal muscular atrophy. These approved gene therapy products utilize AAV capsids isolated from natural sources as the delivery vehicle. The sequence and structural diversity of AAV capsid genes contribute to variability in viral tropism, antigenicity, and packaging efficiency that is observed between viral clades. Discovering novel capsids with an array of tissue tropisms is necessary to advance and expand the gene therapy platform.

In the last two decades, AAV engineering through modification of capsid proteins to confer increased tropism to a particular tissue type or to evade anti-AAV neutralizing antibodies has been a major avenue of capsid development. However, isolation of AAV from natural sources such as from tissue, blood, or viral preparations cultured from different animals has remained the primary method for identifying novel ATVs that are suitable for clinical applications. Anti-AAV antibodies have been found in a variety of mammalian sources indicating that the AAV reservoir is vast.

AAVs are among the most effective vector candidates for gene therapy due to their low immunogenicity and non-pathogenic nature. However, despite allowing for efficient gene transfer, the AAV vectors currently used in the clinic can be hindered by preexisting immunity to the virus and restricted tissue tropism. New and more effective AAV vectors are needed.

### SUMMARY OF THE INVENTION

In one aspect, provided herein is a method of delivering of a transgene to one or more target cells of the central nervous system (CNS) of a subject comprising administering to the subject a recombinant adeno-associated virus (AAV) vector comprising an AAVrh91 capsid and a vector genome comprising the transgene operably linked to regulatory sequences that direct expression of the transgene in the target cells of the CNS. In certain embodiments, the target cells of the CNS are parenchymal cells, cells of the choroid plexus, ependymal cells, astrocytes, and/or and neurons, optionally neurons of the cortex, hippocampus, and/or striatum. In certain embodiments, the transgene encodes a secreted gene product. In certain embodiments, the AAV vector is delivered intrathecally, optionally via intra-cisterna magna (ICM) injection. In certain embodiments, the AAV vector is delivered via intraparenchymal administration.

In one aspect, provided herein is a method for improving delivery of a transgene to the liver of a subject following intrathecal administration of an AAV vector comprising the transgene comprising administering to the subject via ICM injection a recombinant AAV vector comprising an AAVrh91 capsid and a vector genome comprising the transgene operably linked to regulatory sequences that direct expression of the transgene in the target cells of the liver, wherein the levels of transduction of the liver are increased relative to those achieved with an AAV vector having an AAV1, AAV9, and/or AAV6.2 capsid.

In one aspect, provided herein is a method for detargeting the liver and/or reducing liver toxicity following systemic administration of an AAV vector to a subject comprising administering to the subject via intravenous injection a recombinant AAV vector comprising an AAVrh91 capsid and a vector genome comprising a transgene operably linked to regulatory sequences that direct expression of the transgene in cells of the liver, wherein levels of transduction of the liver and/or liver toxicity observed following administration of the AAV vector are reduced relative to an AAV vector having an AAV1, AAV8, and/or AAV9 capsid. In certain embodiments, the AAVrh91 capsid comprises a capsid protein comprising the amino acid sequence of SEQ ID NO: 2. In certain embodiments, the AAVrh91 capsid comprises a capsid protein produced by expression of a nucleotide sequence of SEQ ID NO: 1 or 3, or a sequence sharing at least 90%, at least 95%, at least 97%, at least 98% or at least 99% a nucleotide sequence of SEQ ID NO: 1 or 3. In certain embodiments, the AAVrh91 capsid comprises a capsid protein wherein the capsid protein is encoded by a nucleotide sequence of SEQ ID NO: 1 or 3. In certain embodiments, the AAVrh91 capsid comprises capsid proteins comprising: (1) a heterogeneous population of AAVrh91 vp1 proteins selected from: vp1 proteins produced by expression from a nucleic acid sequence which encodes the predicted amino acid sequence of 1 to 736 of SEQ ID NO: 2, vp1 proteins produced from SEQ ID NO: 1 or 3, or vp1 proteins produced from a nucleic acid sequence at least 70% identical to SEQ ID NO: 1 or 3 which encodes the predicted amino acid sequence of 1 to 736 of SEQ ID NO: 2, a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 2, vp2 proteins produced from a sequence comprising at least nucleotides 412 to 2208 of SEQ ID NO: 1 or 3, or vp2 proteins produced from a nucleic acid sequence at least 70% identical to at least nucleotides 412 to 2208 of SEQ ID NO: 1 or 3 which encodes the predicted amino acid sequence of at least about amino acids 138 to 736 of SEQ ID NO: 2, a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 2, vp3 proteins produced from a sequence comprising at least nucleotides 607 to 2208 of SEQ ID NO: 1 or 3, or vp3 proteins produced from a nucleic acid sequence at least 70% identical to at least nucleotides 607 to 2208 of SEQ ID NO: 1 or 3 which encodes the predicted amino acid sequence of at least about amino acids 203 to 736 of SEQ ID NO: 2; and/or (2) a heterogeneous population of vp1 proteins which are the product of a nucleic acid sequence encoding the amino acid sequence of SEQ ID NO: 2, a heterogeneous population of vp2 proteins which are the product of a nucleic acid sequence encoding the amino acid sequence of at least about amino acids 138 to 736 of SEQ ID NO: 2, and a heterogeneous population of vp3 proteins which are the product of a nucleic acid sequence encoding at least amino acids 203 to 736 of SEQ ID NO: 2, wherein: the vp1, vp2 and vp3 proteins contain subpopulations with amino acid modifications comprising at least two highly deamidated asparagines (N) in asparagine-glycine pairs in SEQ ID NO: 2 and optionally further comprising subpopulations comprising other deamidated amino acids, wherein the deamidation results in an amino acid change.

In one aspect, provided herein is a recombinant AAV production system useful for producing a recombinant AAV, wherein the production system comprises: (a) a nucleotide sequence encoding an AAV capsid protein having an amino acid substitution at one or more of position 418, 547, 584, 588, 598, and/or 642 (when aligned with SEQ ID NO: 2); (b) a nucleic acid molecule suitable for packaging into an AAV capsid, said nucleic acid molecule comprising at least one AAV inverted terminal repeat (ITR) and a non-AAV nucleic acid sequence encoding a gene product operably linked to sequences which direct expression of the product in a host cell; and (c) sufficient AAV rep functions and helper functions to permit packaging of the nucleic acid molecule into the AAV capsid. In certain embodiments, the nucleotide sequence of (a) encodes a clade A capsid protein having a substitution at one or more of the recited positions. In certain embodiments, the nucleotide sequence of (a) encodes an AAV1, AAVhu48R3, AAVhu48, AAVhu44, AAV.VR-355, AAV.VR-195, AAV6, or AAV6.2 capsid protein having an amino acid substitution at one or more of the recited positions. In certain embodiments, the nucleotide sequence of (a) encodes the amino acid sequence of a capsid protein having one or more amino substitutions selected from: Asp at position 418, Asn at position 547, Leu at position 584, Asn at position at 588, Val at position 598, and His at position 642. In certain embodiments, the nucleotide sequence of (a) encodes the amino acid sequence of SEQ ID NO: 8 (AAV1) having amino acid substitutions at Glu418, Ser547, Phe584, Ser588, Ala598, and/or Asn642, and wherein the encoded amino acid sequence is at least 95% identical or at least 99% identical to SEQ IN NO: 8. In certain embodiments, the nucleotide sequence of (a) encodes the amino acid sequence of SEQ ID NO: 8 (AAV1) having one or more amino substitutions selected from: Asp at position 418, Asn at position 547, Leu at position 584, Asn at position at 588, Val at position 598, and His at position 642, and wherein the encoded amino acid sequence is at least 95% identical or at least 99% identical to SEQ IN NO: 8. In certain embodiments, the production system comprises human embryonic kidney 293 cells.

In one aspect, provided herein is a method of generating a recombinant AAV comprising the steps of culturing a host cell containing: (a) a nucleic acid molecule encoding an AAV capsid protein having an amino acid substitution at one or more of position 418, 547, 584, 588, 598, and 642 (when aligned with SEQ ID NO: 2); (b) a functional rep gene; (c) a minigene comprising an AAV 5′ ITR, an AAV 3′ ITR, and a transgene; and (d) sufficient helper functions to permit packaging of the minigene into an AAV capsid. In certain embodiments, the generated recombinant AAV has improved production yields and/or altered cell or tissue tropism relative to an unmodified capsid protein. In certain embodiments, the generated recombinant AAV transduces cells of the CNS at higher levels relative to an unmodified capsid protein. In certain embodiments, the nucleotide sequence of (a) encodes an clade A capsid protein having a substitution at one or more of the recited positions. In certain embodiments, the nucleotide sequence of (a) encodes an AAV1, AAVhu48R3, AAVhu48, AAVhu44, AAV.VR-355, AAV.VR-195, AAV6, or AAV6.2 capsid having one or more of the recited substitutions. In certain embodiments, the nucleotide sequence of (a) encodes the amino acid sequence of a capsid protein having one or more amino substitutions selected from: Asp at position 418, Asn at position 547, Leu at position 584, Asn at position at 588, Val at position 598, and His at position 642. In certain embodiments, the nucleotide sequence of (a) encodes the amino acid sequence of SEQ ID NO: 8 (AAV1) having amino acid substitutions at Glu418, Ser547, Phe584, Ser588, Ala598, and/or Asn642, and wherein the encoded amino acid sequence is at least 95% identical or at least 99% identical to SEQ IN NO: 8. In certain embodiments, the nucleotide sequence of (a) encodes the amino acid sequence of SEQ ID NO: 8 (AAV1) having one or more amino substitutions selected from: Asp at position 418, Asn at position 547, Leu at position 584, Asn at position at 588, Val at position 598, and His at position 642, and wherein the encoded amino acid sequence is at least 95% identical or at least 99% identical to SEQ IN NO: 8.

In one aspect, provided herein is a recombinant AAV vector for use in delivering a transgene to one or more target cells of the central nervous system (CNS) of a subject, wherein the recombinant AAV vector comprises an AAVrh91 capsid and a vector genome comprising the transgene operably linked to regulatory sequences that direct expression of the transgene in the target cells of the CNS. In certain embodiments, the target cells of the CNS are parenchymal cells, cells of the choroid plexus, ependymal cells, astrocytes, and/or and neurons, optionally neurons of the cortex, hippocampus, and/or striatum. In certain embodiments, the transgene encodes a secreted gene product. In certain embodiments, the AAV vector is administered intrathecally, optionally via intra-cistema magna (ICM) injection. In certain embodiments, the AAV vector is delivered via intraparenchymal administration.

In one aspect, provided herein is a recombinant AAV vector for use in delivering a transgene to the liver of a subject following intrathecal administration of an AAV vector comprising the transgene, wherein the recombinant AAV vector comprises an AAVrh91 capsid and a vector genome comprising the transgene operably linked to regulatory sequences that direct expression of the transgene in the target cells of the liver, wherein the levels of transaction of the liver are increased relative to those achieved with a AAV vector having an AAV1, AAV9, and/or AAV6.2 capsid

In one aspect, provided herein is a recombinant AAV vector for use in detargeting the liver and/or reducing liver toxicity following systemic administration of the AAV vector to a subject, wherein the recombinant AAV vector comprises an AAVrh91 capsid and a vector genome comprising a transgene operably linked to regulatory sequences that direct expression of the transgene in cells of the liver, wherein levels of transduction of the liver and/or liver toxicity observed following administration of the AAV vector are reduced relative to an AAV vector having an AAV1, AAV8, and/or AAV9 capsid.

Other aspects and advantages of these compositions and methods are described further in the following detailed description.

### BRIEF DESCRIPTION OF THE FIGURES

FIG. 1 shows a diagram for an AAV-SGA workflow. Genomic DNA was isolated from rhesus macaque tissue samples and screened for the presence of AAV capsid genes. AAV-positive DNA was endpoint diluted and subjected to a further round of PCR. According to a Poisson distribution, the DNA dilution that yields PCR products in no more than 30% of wells contains one amplifiable DNA template per positive PCR 80% of the time. Positive amplicons were sequenced using the Illumina MiSeq 2×150 or 2×250 paired end sequencing platforms and resulting reads were de novo assembled using the SPAdes assembler.

FIG. 2 is a diagram showing the neighbor-joining phylogeny of DNA genome sequences of novel AAV natural isolates and representative clade controls.

FIG. 3A-FIG. 3D show an alignment for nucleic acid sequences for AAVrh91 (SEQ ID NO: 1), AAVrh91eng (SEQ ID NO: 3), AAV6.2 (SEQ ID NO: 5), and AAV1 (SEQ ID NO: 7) capsids.

FIG. 4A-FIG. 4B show an alignment of the amino acid sequences for AAVrh91 (SEQ ID NO: 2), AAV6.2 (SEQ ID NO: 6), and AAV1 (SEQ ID NO: 8) capsids.

FIG. 5A-FIG. 5B show analyses of in vitro transduction of Huh7 (FIG. 15A) and HEK293 cells (FIG. 15B) to evaluate vector yields. AAV.CB7.CI.ffLuc transgene expression was assayed by luciferase activity assay. Vector was administered to cells at a concentration of 1×10¹⁰ GC/mL. n=3. Data depicted as mean and SD; *p<0.01.

FIG. 6A-FIG. 6B show analyses of vector production yields after purification. (FIG. 6A) Average vector yields based on capsid. (FIG. 6B) Clade A capsid vector yields based on transgene. Data depicted as mean and SEM; *p<0.01.

FIG. 7A-FIG. 7C show results of mass spectrometry analyses of AAVrh91 vector preparations.

FIG. 8A-FIG. 8D show eGFP transgene biodistribution in mouse tissues 14 days post injection. (FIG. 8A and FIG. 8B) C57BL/6 mice were injected IV at a dose of 1×10¹² GC per mouse with AAV capsids containing CB7.CI.eGFP.WPRE.RBG (n=5). (FIG. 8C and FIG. 8D) C57BL/6 mice were injected intracerebroventricularly ICV at a dose of 1×10¹¹ GC per mouse with various AAV capsids (clade A vectors dosed at 6.9×10¹⁰ GC/mouse) containing CB7.CI.eGFP.WPRE.RBG (n=5). Values are expressed as mean±SD; *p<0.01, **p<0.001.

FIG. 9A-FIG. 9B show analysis of β-galactosidase expression in skeletal muscle following IM delivery of AAV vectors. Mice were administered 3×10⁹ GC of vectors having various capsids and containing the pAAV.CMV.LacZ transgene. On day 20, muscle tissue was harvested, and transgene expression was evaluated by X-gal staining (darker staining).

FIG. 10 shows levels of mAb in serum following IM delivery of various AAV vectors. B6 mice were administered 1×10¹¹ GC of vector expressing the 3D6 antibody under the tMCK promoter.

FIG. 11 shows yields (relative to AAV8) for vectors expressing 3D6 or LacZ transgenes.

FIG. 12 shows experimental designs for the pooled barcoded vector studies in NHP (data shown in FIG. 13A-FIG. 13D). Five novel capsids and five controls (AAVrh.90, AAVrh91, AAVrh.92, AAVrh.93, AAVrh91.93, AAV8, AAV6.2, AAVrh32.33, AAV7, and AAV9) were packaged with a modified ATG-depleted GFP transgene with unique 6 bp barcodes. Vectors were pooled at equal quantities and injected IV or ICM in cynomolgous macaques (total doses: 2×10¹³ GC/kg IV and 3×10¹³ GC ICM). The IV injected animal was seronegative for AAV6, AAV8, and AAVrh32.33 at baseline and had neutralizing antibody titers of 1:5 and 1:10 against AAV7 and AAV9, respectively.

FIG. 13A-FIG. 13D are graphs showing RNA expression analysis of barcoded capsids after IV delivery (FIG. 13A and FIG. 13B) and ICM delivery (FIG. 13C and FIG. 13D). IV Administration—2×10¹³ GC/kg total dose, necropsy at day 30. ICM Administration −3×10¹³ GC/animal, necropsy at day 30. Barcode frequencies in each tissue RNA sample were normalized to frequencies in injection input material such that each barcode had an equivalent representation (10%) in the mixtures. Input quantities of ten vectors ranged from 8.5-12% Values are expressed as mean±SEM, **p<0.001.

FIG. 14 shows GFP expression microscopy analysis following IV delivery of AAVrh91 and AAV6.2 vectors in mice at 14 dpi. C57BL/6 mice were injected IV with vectors containing the pAAV.CB7.CI.eGFP.WPRE.RBG transgene at a dose of 1×10¹² GC/mouse, n=5. Representative images from liver, heart, brain, and muscle showing GFP transgene expression by direct fluorescence.

FIG. 15 shows GFP expression microscopy analysis of novel AAV vectors following ICV delivery in mice at 14 dpi. C57BL/6 mice were injected ICV with vectors containing the pAAV.CB7.CI.eGFP.WPRE.RBG transgene, n=5. Representative images of GFP fluorescence of clade A vector transduced ependymal cells and choroid plexus are shown. Scale bars: 100 μm.

FIG. 16A-FIG. 16C show immunohistochemistry for GFP transgene expression after ICM delivery in rhesus macaque CNS tissues. Animals were administered 1.6×10¹³ GC vectors containing the pAAV.CB7.CI.eGFP.WPRE.rBG transgene via ICM injection. Transduction of vector in brain (FIG. 16A) lateral ventricle (FIG. 16B) and spinal cord (FIG. 16C) was evaluated 28-31 dpi. n=2 per group. Animal IDs in top right corner. Scale bars: 100 μm.

FIG. 17 shows immunohistochemistry for GFP transgene expression after ICM delivery in rhesus macaque liver and heart tissues. Animals were administered 1.6×10¹³ GC of vectors containing the pAAV.CB7.CI.eGFP.WPRE.rBG transgene via ICM injection. Transduction of vector in liver and heart was evaluated at 28-31 dpi. n=2 per group. Animal IDs in top right corner. Scale bars: 100 μm.

FIG. 18A-FIG. 18E show an analysis of cellular tropism of novel vector AAVrh91 after ICM delivery in NHP. Animals were given 1.6×10¹³ GC vectors containing the pAAV.CB7.CI.eGFP.WPRE.rBG transgene via ICM injection. Transduction of vector was evaluated 28-31 dpi. n=2 NHP per capsid. Quantification of average GFP expression relative to AAV9 in astrocytes (FIG. 18C) and neurons (FIG. 18D) in brain sections identified in FIG. 18A and FIG. 18B. (FIG. 18E) Quantification of AAVrh91 and AAV9 neuron transduction in subregions of the brain. Ctx.: cortex, Fr.: frontal, Temp.: temporal, Par.: parietal, Occ.: occipital, Str.: striatum, Thal.: thalamus, Hip.: hippocampus. AAVrh91 transduces a higher proportion of neurons and astrocytes than AAV9 in the NHP brain following ICM delivery.

FIG. 19A-FIG. 19C show biodistribution of vector transgenes following ICM delivery of AAVrh91, AAV1, and AAV9 capsids to NHP. Animals were administered 1.6×10¹³ GC vectors containing the pAAV.CB7.CI.eGFP.WPRE.rBG transgene. n=2 NHP per capsid. Biodistribution of vector was evaluated by qPCR 28-31 dpi in the cortex (FIG. 19A) and non cortical regions of the brain (FIG. 19B), and spinal cord (FIG. 19C). Values reported as mean and SEM. Animals: AAVrh91 (1409201 and 1407088), AAV1 (RA3654 and RA3583), AAV9 (1408266 and 1409029).

FIG. 20 shows average GC levels in all CNS tissues reported in FIG. 19A-FIG. 19C grouped by animal. *p<0.05, **p<0.01, ****p<0.0001. Animals: AAVrh91 (1409201 and 1407088), AAV1 (RA3654 and RA3583), AAV9 (1408266 and 1409029).

FIG. 21A-FIG. 21B show seroprevalence of AAVrh91 in the human population. Anti-capsid neutralizing antibodies (NAbs) against AAV2, AAV8, AAVrh32.33, and AAVrh91 were evaluated on 50 random human serum samples. (FIG. 21A) Seroprevalence of NAbs to the various capsids and (FIG. 21B) magnitude of NAb response.

FIG. 22A-FIG. 22F show biodistribution of AAV1, AAV8, AAV9, and AAVrh91 following IV administration in C57BL/6J mice. Adult C57BL/6J mice (n=5/group) were injected by IV with 10¹¹ or 10¹² GC/mouse of AAV1, AAV8, AAV9, or AAVrh91 expressing GFP from the CB7 promoter. Mice were necropsied at 21 days post-vector administration. Liver (FIG. 22A), spleen (FIG. 22B), heart (FIG. 22C), skeletal muscle (gastroenemius; FIG. 22D), brain (FIG. 22E), and spinal cord (FIG. 22F) were harvested for evaluation of vector genome copies and vector-derived RNA transcript by qPCR.

FIG. 23A-FIG. 23D show enhanced heart and skeletal muscle, and reduced liver, gene expression with AAVrh91 compared to AAV9 in rhesus macaques. Rhesus macaques were administered IV with 5×10¹³ GC/kg of AAV9 or AAVrh91.CB7.eGFP (n=3/group). Animals were necropsied at day 21 post-vector administration. (FIG. 23A) DNA and (FIG. 23B) RNA were extracted and vector-derived sequences were quantified by qPCR. GFP protein expression was determined by ELISA (FIG. 23C) or IHC with images quantified (FIG. 23D).

FIG. 24A-FIG. 24C show enhanced skeletal muscle transduction with AAVrh91 across the majority of muscle groups in rhesus macaques. Rhesus macaques were administered IV with 5×10¹³ GC/kg of AAV9 or AAVrh91.CB7.eGFP (n=3/group). Animals were necropsied at day 21 post-vector administration and samples from 13 skeletal muscle groups were harvested. (FIG. 24A) DNA and (FIG. 24B) RNA were extracted and vector-derived sequences were quantified by qPCR. GFP protein expression was determined by ELISA (FIG. 24C). For each muscle group, left set of dots is AAV9 and the right set of dots is AAVrh91.

FIG. 25 shows liver degeneration and individual cell necrosis following IV administration with AAV9 and AAVrh91. Rhesus macaques were administered IV with 5×10¹³ GC/kg of AAV9 or AAVrh91.CB7.eGFP (n=3/group).

FIG. 26A-FIG. 26B show clinical pathology evaluation of rhesus macaques following IV administration with AAV9 and AAVrh91. Rhesus macaques were administered IV with 5×10¹³ GC/kg of AAV9 or AAVrh91.CB7.eGFP (n=3/group). Blood samples for evaluation of (FIG. 26A) ALT, AST, alkaline phosphatase, GGT, total bilirubin, (FIG. 26B) prothrombin time (PT), APTT, and platelet count were taken throughout the in-life phase of the study. Animals were necropsied at day 21 post-vector administration.

FIG. 27A-FIG. 27B show clinical pathology evaluation of rhesus macaques following ICM administration with AAV9 and AAVrh91. Rhesus macaques were administered ICM with 3×10¹³ GC/kg of AAV9 or AAVrh91.CB7.eGFP (n=3/group). Blood samples for evaluation of (FIG. 27A) AST, ALT, alkaline phosphatase, GGT, total bilirubin, (FIG. 27B) prothrombin time (PT), APTT, platelet count, CSF white blood cell count were taken throughout the in-life phase of the study. Animals were necropsied at day 14 post-vector administration.

FIG. 28A-FIG. 28C show biodistribution following ICM administration with AAV9 and AAVrh91. Rhesus macaques were administered ICM with 3×10¹³ GC/kg of AAV9 or AAVrh91.CB7.eGFP. Necropsy was performed on day 14.

FIG. 29A-FIG. 29I show quantification of GFP positive sensory neurons in DRG. Rhesus macaques were administered ICM with 3×10¹³ GC/kg of AAV9 or AAVrh91.CB7.eGFP (n=3/group). (FIG. 29A) Immunohistochemistry was performed to detect GFP in tissue sections, and slides were evaluated using imaging software. Data for analysis of cervical segment DRG is shown as GFP+ cells/mm² (FIG. 29B and FIG. 29C) and % GFP positive area (FIG. 29D and FIG. 29E). Data for analysis of lumbar segment DRG is shown as GFP+ cells/mm² (FIG. 29F and FIG. 29G) and % GFP positive area (FIG. 29H and FIG. 29I).

FIG. 30A-FIG. 30G show quantification of GFP positive motor neurons in spinal cord segments. Rhesus macaques were administered ICM with 3×10¹³ GC/kg of AAV9 or AAVrh91.CB7.eGFP (n=3/group). (FIG. 30A) Immunohistochemistry was performed to detect GFP in tissue sections, and slides were evaluated using imaging software. GFP positive neurons were manually counted in ventral horn of cervical segment (FIG. 30B and FIG. 30C), ventral horn of thoracic segment (FIG. 30D and FIG. 30E), and ventral horn of lumbar segment (FIG. 30F and FIG. 30G).

FIG. 31A-FIG. 31C show quantification of GFP positive cell in neurons in occipital cortex. Rhesus macaques were administered ICM with 3×10¹³ GC/kg of AAV9 or AAVrh91.CB7.eGFP (n=3/group). (FIG. 31A) Immunohistochemistry was performed to detect GFP in tissue sections, and slides were evaluated using imaging software. GFP positive neurons were manually counted (FIG. 31B and FIG. 31C).

FIG. 32 shows nerve conduction velocity evaluations following ICM administration with AAV9 and AAVrh91. Rhesus macaques were administered ICM with 3×10¹³ GC/kg of AAV9 or AAVrh91.CB7.2.10mAb (n=3/group). Evaluations were performed at baseline and during in-life phase.

FIG. 33A-FIG. 33B show concentrations of transgene in CSF and serum following ICM administration with AAV9 and AAVrh91. Rhesus macaques were administered ICM with 3×10¹³ GC/kg of AAV9 or AAVrh91.CB7.2.10mAb (n=3/group). Serum (FIG. 33A) and CSF (FIG. 33B) were monitored for 2.10mAb expression.

FIG. 34A-FIG. 34B show biodistribution following ICM administration with AAV9 and AAVrh91. Rhesus macaques were administered ICM with 3×10¹³ GC/kg of AAV9 or AAVrh91.CB7.2.10mAb (n=3/group). Necropsy was performed on day 90 post-vector administration.

FIG. 35A-FIG. 35F shows structural differences between the AAVrh91 and AAV1 capsids. The structure for AAVrh91 was solved using cryoEM to a resolution of 2.33 Å and compared to a published structure for AAV1 (6JCR). Amino acids in VP3 which differ between the two capsids are located at position (FIG. 35A) 418, (FIG. 35B) 547, (FIG. 35C) 584, (FIG. 35D) 588, (FIG. 35E) 598, and (FIG. 35F) 642, and are shown in their structural context. Amino acids at a specified position are colored black, and all other amino acids are colored grey. Amino acids forming close contacts with the specified residue receive a text label. Intra-chain and inter-chain contacts for each residue are shown with a letter designation of A or B. FIG. 35A shows the AAVrh91 Asp 418 amino acid residue in its structural context, as compared to the AAV1 Glu 418 in the same position. Amino acids Arg 308, Lys 310, and Glu 686, which are in close proximity to the residues at position 418 are also labeled. Amino acids at position 418 are colored black, and all other amino acids are colored grey. All labeled amino acids are located on the same polypeptide chain, and form intra-chain contacts with the residue at position 418. FIG. 35B shows the AAVrh91 Asn 547 amino acid residue in its structural context, as compared to the AAV1 Ser 547 in the same position. Amino acids at position 547 are colored black, and all other amino acids are colored grey. FIG. 35C shows the AAVrh91 Leu 584 amino acid residue in its structural context, as compared to the AAV1 Phe 584 in the same position. Amino acids Arg 485, Arg 488, Lys 528, Glu 531, Phe 534, Thr 574, and Glu575, which are in close proximity to the residues at position 584 are also labeled. Amino acids at position 584 are colored black, and all other amino acids are colored grey. Inter-chain contacts are shown with a letter designation of A or B. Amino acids at position 584 are labeled with an A, and the surrounding residues, labeled with a B, are found on an adjacent polypeptide chain. FIG. 35D shows the AAVrh91 Asn 588 amino acid residue in its context at the tip of the 3-fold spike structure, as compared to the AAV1 Ser 588 in the same position. Amino acids at position 588 are colored black, and all other amino acids are colored grey. FIG. 35E shows the AAVrh91 Val 598 amino acid residue in its structural context, as compared to the AAV1 Ala 598 in the same position. Amino acids Tyr 484, Val 580, Val 596, Met 599, and Leu 602, which are in close proximity to the residues at position 598 are also labeled. Amino acids at position 598 are colored black, and all other amino acids are colored grey. Intra-chain and inter-chain contacts for each residue are shown with a letter designation of A or B. Amino acids labeled with an A are on the same polypeptide chain as the amino acid at position 598, and amino acids labeled with a B are on an adjacent chain. FIG. 35F shows the AAVrh91 His 642 amino acid residue in its structural context, as compared to the AAV1 Asn 642 in the same position. Amino acids Tyr 349, Tyr 414, Glu 417, and Lys 641, which are in close proximity to the residues at position 642 are also labeled. Amino acids at position 642 are colored black, and all other amino acids are colored grey. All labeled amino acids are located on the same polypeptide chain, and form intra-chain contacts with the residue at position 642.

FIG. 36A-FIG. 36B show AAV vector yield comparisons for trans plasmids having the AAVrh91 coding sequence and an engineered AAVrh91 coding sequence (AAVrh91eng). For each construct, plasmids were re-transformed, and four clones were randomly picked for individual triple-transfections in 12-well plates. Vector yields were determined by two methods—(FIG. 36A) qPCR for production titers (FIG. 36B) Huh7 transduction for infectious titers. Each experiment was conducted twice—Repeat 1 and Repeat 2.

FIG. 37A-FIG. 37C show AAV vector yield comparisons for trans plasmids having additional regulatory elements. Trans plasmids that included a WPRE and/or bGH polyA were generated (FIG. 37A). Vector yields were determined by two methods—qPCR (FIG. 37B) for production titers and Huh7 (FIG. 37C) transduction for infectious titers. Each experiment was conducted twice—Repeat 1 and Repeat 2.

### DETAILED DESCRIPTION OF THE INVENTION

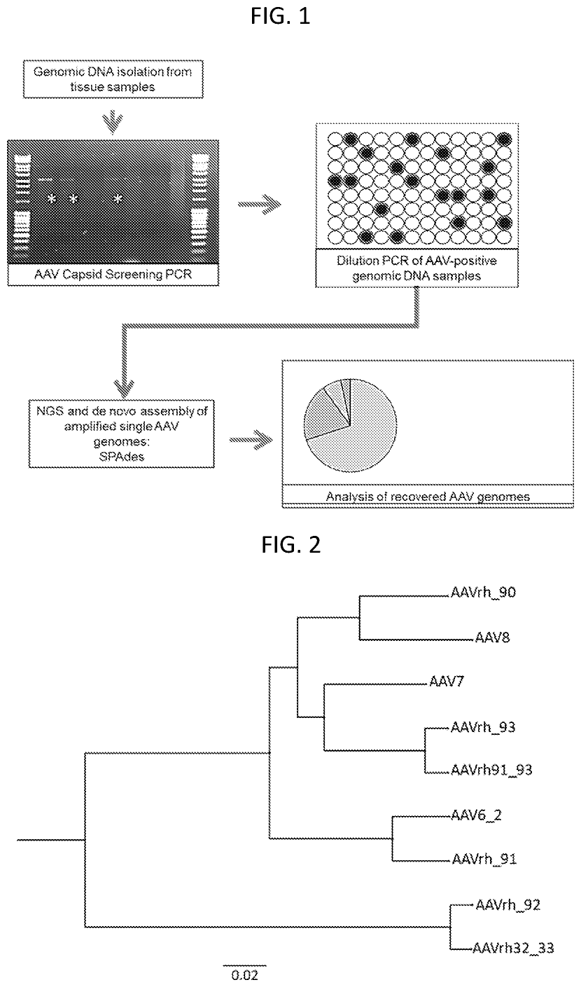

The genetic variation of AAVs in their natural mammalian hosts was explored by using AAV single genome amplification, a technique used to accurately isolate individual AAV genomes from within a viral population (FIG. 1). Described herein is the isolation of novel AAV sequences from rhesus macaque tissues that can be categorized in various clades. The novel capsid sequences described were used to produce gene delivery vectors. We assessed the biological properties of the natural isolate-derived AAV vectors in mice after intravenous (IV) and intracerebroventricular (ICV) delivery, and in NHP following IV and intra-cistema magna (ICM) delivery. The results identified both clade-specific and variable transduction patterns of the new AAV variants when compared to their prototypical clade member controls.

Provided herein is a recombinant AAVrh91 vector having an AAVrh91 capsid and a nucleic acid encoding a transgene under the control of regulatory sequences, which direct expression thereof following delivery to a subject. The rAAVrh91 capsid contains proteins independently having the amino acid sequence of SEQ ID NO: 2. Compositions containing these vectors are provided. The methods described herein are directed to use of rAAV to target tissues of interest for treatment of various conditions.

In certain embodiments, provided herein is a vector comprising an AAVrh91 capsid well suited for delivery of a transgene to the central nervous system. In certain embodiments, intrathecal delivery is desired, including, e.g., delivery to the brain via ICM delivery. In certain embodiments, vectors comprising the AAVrh91 capsid are well suited for delivery of a transgene to the smooth muscle. In certain embodiments, vectors comprising the AAVrh91 capsid are well suited for delivery of a transgene to heart tissue. In other embodiments, vectors comprising the AAVrh91 capsid are well suited for delivery to skeletal (striated) muscle. In certain embodiments, AAVrh91 vectors may be delivered systemically or targeted via a route of administration suitable to target these tissues. In certain embodiments, administration of a AAVrh91 vector to the CNS also results in transgene delivery to one or more peripheral organs (including, e.g., the heart and/or liver).

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. The following definitions are provided for clarity only and are not intended to limit the claimed invention. As used herein, the terms “a” or “an”, refers to one or more, for example, “a host cell” is understood to represent one or more host cells. As such, the terms “a” (or “an”), “one or more,” and “at least one” are used interchangeably herein. As used herein, the term “about” means a variability of 10% from the reference given, unless otherwise specified. While various embodiments in the specification are presented using “comprising” language, under other circumstances, a related embodiment is also intended to be interpreted and described using “consisting of” or “consisting essentially of” language.

With regard to the following description, it is intended that each of the compositions herein described, is useful, in another embodiment, in the methods of the invention. In addition, it is also intended that each of the compositions herein described as useful in the methods, is, in another embodiment, itself an embodiment of the invention.

A “recombinant AAV” or “rAAV” is a DNAse-resistant viral particle containing two elements, an AAV capsid and a vector genome containing at least non-AAV coding sequences packaged within the AAV capsid. Unless otherwise specified, this term may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. In certain embodiments, the ITRs are from AAV2, a different source AAV than the capsid, or other than full-length ITRs may be selected. In certain embodiments, the ITRs are from the same AAV source as the AAV which provides the rep function during production or a transcomplementing AAV. Further, other ITRs may be used. Further, the vector genome contains regulatory sequences which direct expression of the gene products. Suitable components of a vector genome are discussed in more detail herein. The vector genome is sometimes referred to herein as the “minigene”.

The term “expression cassette” refers to a nucleic acid molecule which comprises a transgene sequences and regulatory sequences therefore (e.g., promoter, enhancer, polyA), which cassette may be packaged into the capsid of a viral vector (e.g., a viral particle). Typically, such an expression cassette for generating a viral vector contains the transgene sequences flanked by packaging signals of the viral genome and other expression control sequences such as those described herein. For example, for an AAV viral vector, the packaging signals are the 5′ inverted terminal repeat (ITR) and the 3′ ITR. In certain embodiments, the term “transgene” may be used interchangeably with “expression cassette”. In other embodiments, the term “transgene” refers solely to the coding sequences for a selected gene.

A rAAV is composed of an AAV capsid and a vector genome. An AAV capsid is an assembly of a heterogeneous population of vp1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences.

As used herein, the term “heterogeneous population” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins may be at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs. See PCT/US19/019804, filed Feb. 27, 2019, and PCT/US19/019861, filed Feb. 27, 2019, each of which is hereby incorporated by reference.

Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, at least 97%, at least 99%, or up to about 100% deamidated at a referenced amino acid position, as compared to the predicted amino acid sequence at the reference amino acid position. Such percentages may be determined using 2D-gel, mass spectrometry techniques, or other suitable techniques.

Without wishing to be bound by theory, the deamidation of at least highly deamidated residues in the vp proteins in the AAV capsid is believed to be primarily non-enzymatic in nature, being caused by functional groups within the capsid protein which deamidate selected asparagines, and to a lesser extent, glutamine residues. Efficient capsid assembly of the majority of deamidation vp1 proteins indicates that either these events occur following capsid assembly or that deamidation in individual monomers (vp1, vp2 or vp3) is well-tolerated structurally and largely does not affect assembly dynamics. Extensive deamidation in the VP1-unique (VP1-u) region (˜aa 1-137), generally considered to be located internally prior to cellular entry, suggests that VP deamidation may occur prior to capsid assembly.

Without wishing to be bound by theory, the deamidation of N may occur through its C-terminus residue's backbone nitrogen atom conducts a nucleophilic attack to the Asn's side chain amide group carbon atom. An intermediate ring-closed succinimide residue is believed to form. The succinimide residue then conducts fast hydrolysis to lead to the final product aspartic acid (Asp) or iso aspartic acid (IsoAsp). Therefore, in certain embodiments, the deamidation of asparagine (N or Asn) leads to an Asp or IsoAsp, which may interconvert through the succinimide intermediate e.g., as illustrated below.

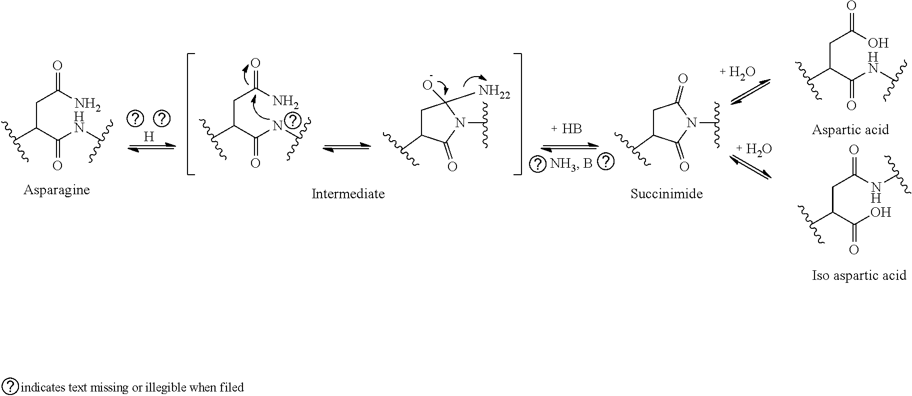

As provided herein, each deamidated N in the VP1, VP2 or VP3 may independently be aspartic acid (Asp), isoaspartic acid (isoAsp), aspartate, and/or an interconverting blend of Asp and isoAsp, or combinations thereof. Any suitable ratio of α- and isoaspartic acid may be present. For example, in certain embodiments, the ratio may be from 10:1 to 1:10 aspartic to isoaspartic, about 50:50 aspartic: isoaspartic, or about 1:3 aspartic: isoaspartic, or another selected ratio.

In certain embodiments, one or more glutamine (Q) may deamidates to glutamic acid (Glu), i.e., α-glutamic acid, γ-glutamic acid (Glu), or a blend of α- and γ-glutamic acid, which may interconvert through a common glutarimide intermediate. Any suitable ratio of α- and γ-glutamic acid may be present. For example, in certain embodiments, the ratio may be from 10:1 to 1:10 α to γ, about 50:50 α:γ, or about 1:3 α:γ, or another selected ratio.

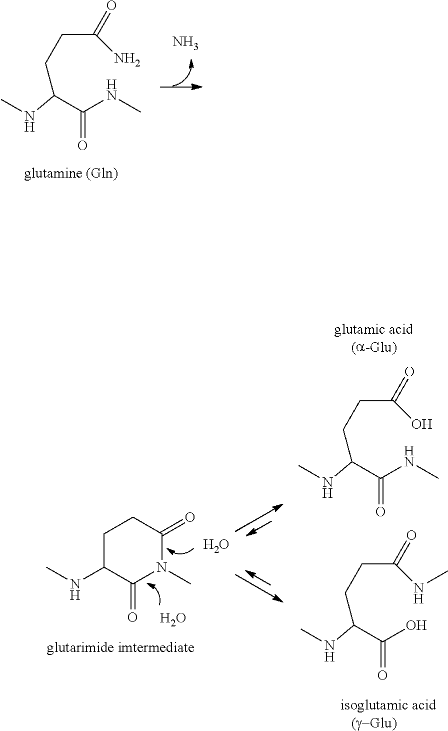

Thus, an rAAV includes subpopulations within the rAAV capsid of vp1, vp2 and/or vp3 proteins with deamidated amino acids, including at a minimum, at least one subpopulation comprising at least one highly deamidated asparagine. In addition, other modifications may include isomerization, particularly at selected aspartic acid (D or Asp) residue positions. In still other embodiments, modifications may include an amidation at an Asp position.

In certain embodiments, an AAV capsid contains subpopulations of vp1, vp2 and vp3 having at least 1, at least 2, at least 3, at least 4, at least 5 to at least about 25 deamidated amino acid residue positions, of which at least 1 to 10%, at least 10 to 25%, at least 25 to 50%, at least 50 to 70%, at least 70 to 100%, at least 75 to 100%, at least 80-100% or at least 90-100% are deamidated as compared to the encoded amino acid sequence of the vp proteins. The majority of these may be N residues. However, Q residues may also be deamidated.

As used herein, “encoded amino acid sequence” refers to the amino acid which is predicted based on the translation of a known DNA codon of a referenced nucleic acid sequence being translated to an amino acid. The following table illustrates DNA codons and twenty common amino acids, showing both the single letter code (SLC) and three letter code (3LC).

| Amino Acid | SLC | 3 LC | DNA codons |
|---|---|---|---|
| Isoleucine | I | Ile | ATT, ATC, ATA |
| Leucine | L | Leu | CTT, CTC, CTA, CTG, TTA, TTG |
| Valine | V | Val | GTT, GTC, GTA, GTG |
| Phenylalanine | F | Phe | TTT, TTC |
| Methionine | M | Met | ATG |
| Cysteine | C | Cys | TGT, TGC |
| Alanine | A | Ala | GCT, GCC, GCA, GCG |
| Glycine | G | Gly | GGT, GGC, GGA, GGG |
| Proline | P | Pro | CCT, CCC, CCA, CCG |
| Threonine | T | Thr | ACT, ACC, ACA, ACG |
| Serine | S | Ser | TCT, TCC, TCA, TCG, AGT, AGC |
| Tyrosine | Y | Tyr | TAT, TAC |
| Tryptophan | W | Trp | TGG |
| Glutamine | Q | Gln | CAA, CAG |
| Asparagine | N | Asn | AAT, AAC |
| Histidine | H | His | CAT, CAC |
| Glutamic acid | E | Glu | GAA, GAG |
| Aspartic acid | D | Asp | GAT, GAC |
| Lysine | K | Lys | AAA, AAG |
| Arginine | R | Arg | CGT, CGC, CGA, CGG, AGA, |
| | | | AGG |
| Stop codons | Stop | | TAA, TAG, TGA |

In certain embodiments, a rAAV has an AAV capsid having vp1, vp2 and vp3 proteins having subpopulations comprising combinations of two, three, four, five or more deamidated residues at the positions set forth in the tables provided herein and incorporated herein by reference.

Deamidation in the rAAV may be determined using 2D gel electrophoresis, and/or mass spectrometry, and/or protein modelling techniques. Online chromatography may be performed with an Acclaim PepMap column and a Thermo UltiMate 3000 RSLC system (Thermo Fisher Scientific) coupled to a Q Exactive HF with a NanoFlex source (Thermo Fisher Scientific). MS data is acquired using a data-dependent top-20 method for the Q Exactive HF, dynamically choosing the most abundant not-yet-sequenced precursor ions from the survey scans (200-2000 m/z). Sequencing is performed via higher energy collisional dissociation fragmentation with a target value of 1e5 ions determined with predictive automatic gain control and an isolation of precursors was performed with a window of 4 m/z. Survey scans were acquired at a resolution of 120,000 at m/z 200. Resolution for HCD spectra may be set to 30,000 at m/z 200 with a maximum ion injection time of 50 ms and a normalized collision energy of 30. The S-lens RF level may be set at 50, to give optimal transmission of the m/z region occupied by the peptides from the digest. Precursor ions may be excluded with single, unassigned, or six and higher charge states from fragmentation selection. BioPharma Finder 1.0 software (Thermo Fischer Scientific) may be used for analysis of the data acquired. For peptide mapping, searches are performed using a single-entry protein FASTA database with carbamidomethylation set as a fixed modification; and oxidation, deamidation, and phosphorylation set as variable modifications, a 10-ppm mass accuracy, a high protease specificity, and a confidence level of 0.8 for MS/MS spectra. Examples of suitable proteases may include, e.g., trypsin or chymotrypsin. Mass spectrometric identification of deamidated peptides is relatively straightforward, as deamidation adds to the mass of intact molecule +0.984 Da (the mass difference between —OH and —NH₂ groups). The percent deamidation of a particular peptide is determined by mass area of the deamidated peptide divided by the sum of the area of the deamidated and native peptides. Considering the number of possible deamidation sites, isobaric species which are deamidated at different sites may co-migrate in a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent on the site of deamidation. It will be understood by one of skill in the art that a number of variations on these illustrative methods can be used. For example, suitable mass spectrometers may include, e.g, a quadrupole time of flight mass spectrometer (QTOF), such as a Waters Xevo or Agilent 6530 or an orbitrap instrument, such as the Orbitrap Fusion or Orbitrap Velos (Thermo Fisher). Suitably liquid chromatography systems include, e.g., Acquity UPLC system from Waters or Agilent systems (1100 or 1200 series). Suitable data analysis software may include, e.g., MassLynx (Waters), Pinpoint and Pepfinder (Thermo Fischer Scientific), Mascot (Matrix Science), Peaks DB (Bioinformatics Solutions). Still other techniques may be described, e.g., in X. Jin et al, Hu Gene Therapy Methods, Vol. 28, No. 5, pp. 255-267, published online Jun. 16, 2017.

In addition to deamidations, other modifications may occur that do not result in conversion of one amino acid to a different amino acid residue. Such modifications may include acetylated residues, isomerizations, phosphorylations, or oxidations.

Modulation of Deamidation: In certain embodiments, the AAV is modified to change the glycine in an asparagine-glycine pair, to reduce deamidation. In other embodiments, the asparagine is altered to a different amino acid, e.g., a glutamine which deamidates at a slower rate; or to an amino acid which lacks amide groups (e.g., glutamine and asparagine contain amide groups); and/or to an amino acid which lacks amine groups (e.g., lysine, arginine and histidine contain amine groups). As used herein, amino acids lacking amide or amine side groups refer to, e.g., glycine, alanine, valine, leucine, isoleucine, serine, threonine, cystine, phenylalanine, tyrosine, or tryptophan, and/or proline. Modifications such as described may be in one, two, or three of the asparagine-glycine pairs found in the encoded AAV amino acid sequence. In certain embodiments, such modifications are not made in all four of the asparagine-glycine pairs. Thus, a method for reducing deamidation of AAV and/or engineered AAV variants having lower deamidation rates. Additionally, or alternatively, one or more other amide amino acids may be changed to a non-amide amino acid to reduce deamidation of the AAV. In certain embodiments, a mutant AAV capsid as described herein contains a mutation in an asparagine-glycine pair, such that the glycine is changed to an alanine or a serine. A mutant AAV capsid may contain one, two or three mutants where the reference AAV natively contains four NG pairs. In certain embodiments, an AAV capsid may contain one, two, three or four such mutants where the reference AAV natively contains five NG pairs. In certain embodiments, a mutant AAV capsid contains only a single mutation in an NG pair. In certain embodiments, a mutant AAV capsid contains mutations in two different NG pairs. In certain embodiments, a mutant AAV capsid contains mutation is two different NG pairs which are located in structurally separate location in the AAV capsid. In certain embodiments, the mutation is not in the VP1-unique region. In certain embodiments, one of the mutations is in the VP1-unique region. Optionally, a mutant AAV capsid contains no modifications in the NG pairs, but contains mutations to minimize or eliminate deamidation in one or more asparagines, or a glutamine, located outside of an NG pair.

In certain embodiments, a method of increasing the potency of a rAAV vector is provided which comprises engineering an AAV capsid which eliminating one or more of the NGs in the wild-type AAV capsid. In certain embodiments, the coding sequence for the “G” of the “NG” is engineered to encode another amino acid. In certain examples below, an “S” or an “A” is substituted. However, other suitable amino acid coding sequences may be selected.

These amino acid modifications may be made by conventional genetic engineering techniques. For example, a nucleic acid sequence containing modified AAV vp codons may be generated in which one to three of the codons encoding glycine in asparagine-glycine pairs are modified to encode an amino acid other than glycine. In certain embodiments, a nucleic acid sequence containing modified asparagine codons may be engineered at one to three of the asparagine-glycine pairs, such that the modified codon encodes an amino acid other than asparagine. Each modified codon may encode a different amino acid. Alternatively, one or more of the altered codons may encode the same amino acid. In certain embodiments, the modified AAVrh91 nucleic acid sequences is be used to generate a mutant rAAV having a capsid with lower deamidation than the native AAVrh91 capsid. Such mutant rAAV may have reduced immunogenicity and/or increase stability on storage, particularly storage in suspension form.

Also provided herein are nucleic acid sequences encoding the AAV capsids having reduced deamidation. It is within the skill in the art to design nucleic acid sequences encoding this AAV capsid, including DNA (genomic or cDNA), or RNA (e.g., mRNA). Such nucleic acid sequences may be codon-optimized for expression in a selected system (i.e., cell type) and can be designed by various methods. This optimization may be performed using methods which are available on-line (e.g., GeneArt), published methods, or a company which provides codon optimizing services, e.g., DNA2.0 (Menlo Park, Calif.). One codon optimizing method is described, e.g., in International Patent Publication No. WO 2015/012924, which is incorporated by reference herein in its entirety. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence and produce a nucleic acid fragment of a codon-optimized coding region which encodes the polypeptide. A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In certain embodiments, AAV capsids are provided which have a heterogeneous population of AAV capsid isoforms (i.e., VP1, VP2, VP3) which contain multiple highly deamidated “NG” positions. In certain embodiments, the highly deamidated positions are in the locations identified below, with reference to the predicted full-length VP1 amino acid sequence. In other embodiments, the capsid gene is modified such that the referenced “NG” is ablated and a mutant “NG” is engineered into another position.

As used herein, the terms “target cell” and “target tissue” can refer to any cell or tissue which is intended to be transduced by the subject AAV vector. The term may refer to any one or more of muscle, liver, lung, airway epithelium, central nervous system, neurons, eye (ocular cells), or heart. In one embodiment, the target tissue is liver. In another embodiment, the target tissue is the heart. In another embodiment, the target tissue is brain. In certain embodiments, the target cell is one or more cell type of the CNS, including but not limited to astrocytes, neurons, ependymal cells, and cells of the choroid plexus. In another embodiment, the target tissue is muscle.

As used herein, the term “mammalian subject” or “subject” includes any mammal in need of the methods of treatment described herein or prophylaxis, including particularly humans. Other mammals in need of such treatment or prophylaxis include dogs, cats, or other domesticated animals, horses, livestock, laboratory animals, including non-human primates, etc. The subject may be male or female.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

As used herein, the term “host cell” may refer to the packaging cell line in which the rAAV is produced from the plasmid. In the alternative, the term “host cell” may refer to the target cell in which expression of the transgene is desired.

### A. THE AAV CAPSID

Provided herein is a novel AAV capsid protein having the vp1 sequence set forth in SEQ ID NO: 2. The AAV capsid consists of three overlapping coding sequences, which vary in length due to alternative start codon usage. These variable proteins are referred to as VP1, VP2 and VP3, with VP1 being the longest and VP3 being the shortest. The AAV particle consists of all three capsid proteins at a ratio of ˜1:1:10 (VP1:VP2:VP3). VP3, which is comprised in VP1 and VP2 at the N-terminus, is the main structural component that builds the particle. The capsid protein can be referred to using several different numbering systems. For convenience, as used herein, the AAV sequences are referred to using VP1 numbering, which starts with aa 1 for the first residue of VP1. However, the capsid proteins described herein include VP1, VP2 and VP3 (used interchangeably herein with vp1, vp2 and vp3). The numbering of the variable proteins of the capsids are as follows:

Nucleotides (nt)
AAVrh91: vp1—nt 1 to 2208; vp2—nt 412 to 2208; vp3—nt 607 to 2208 of SEQ ID NO: 1
AAVrh91eng: vp1—nt 1 to 2208; vp2—nt 412 to 2208; vp3—nt 607 to 2208 of SEQ ID NO: 3

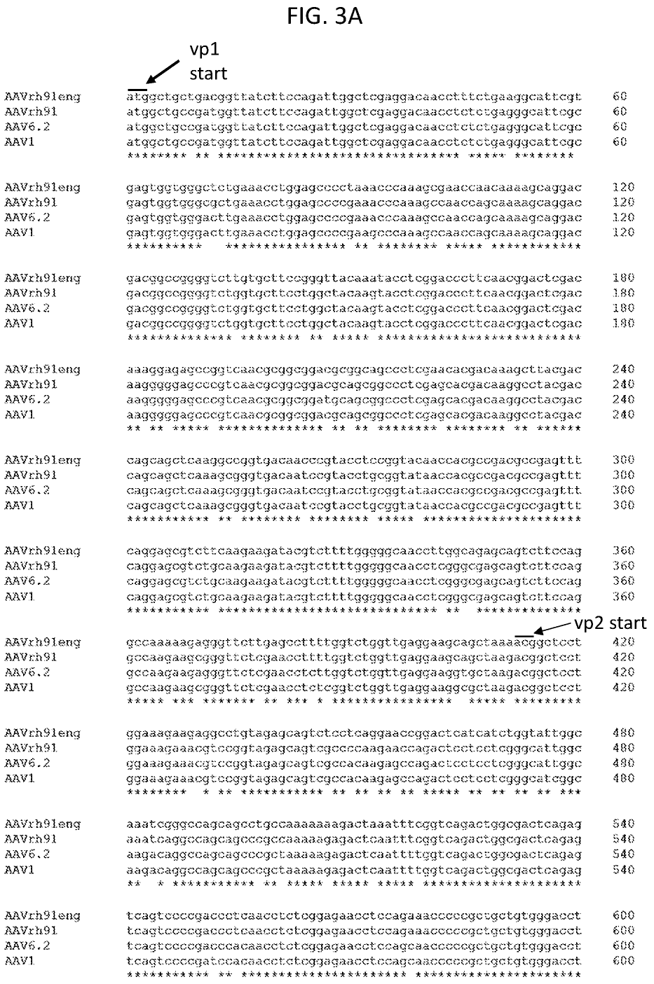

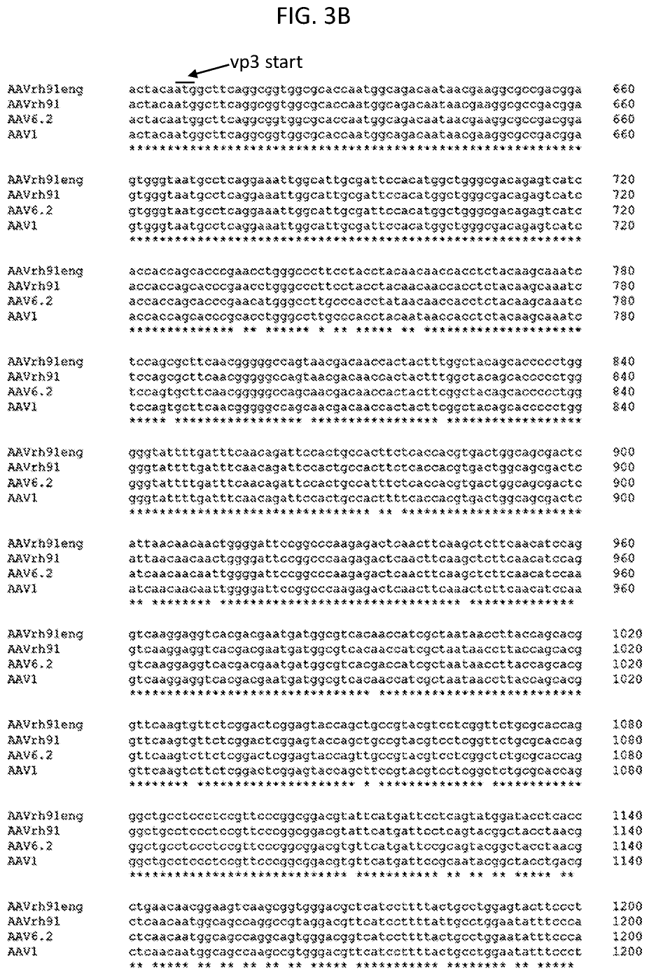

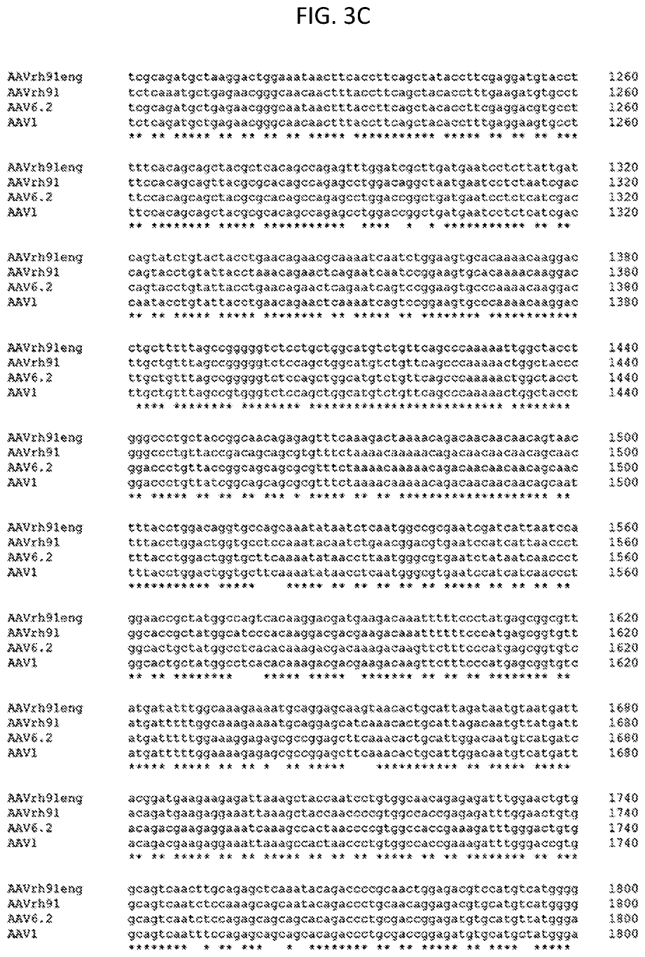

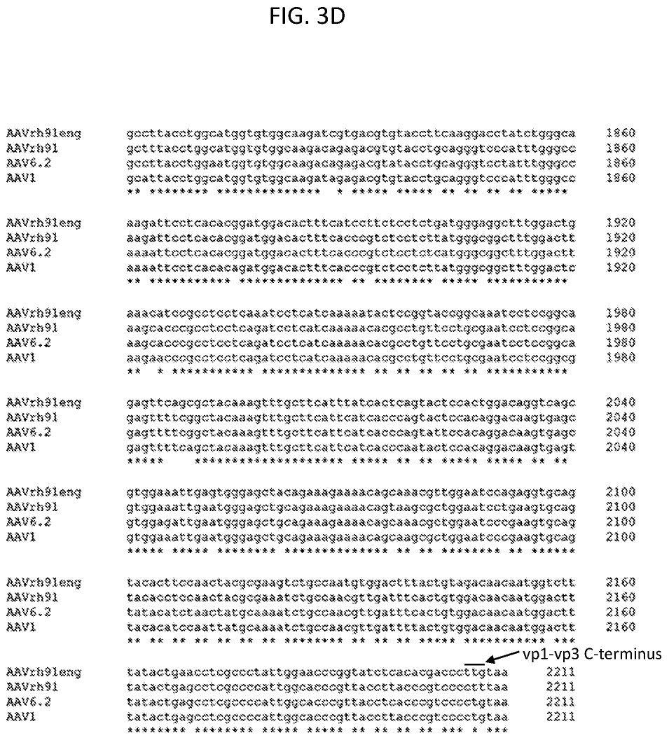

An alignment of the nucleic acid sequences for the capsids described herein is shown in FIG. 3A-FIG. 3D.

Amino Acids (aa)
AAVrh91 and AAVrh91eng: aa vp1—1 to 736; vp2—aa 138 to 736; vp3—aa 203 to 736 of SEQ ID NO: 2.

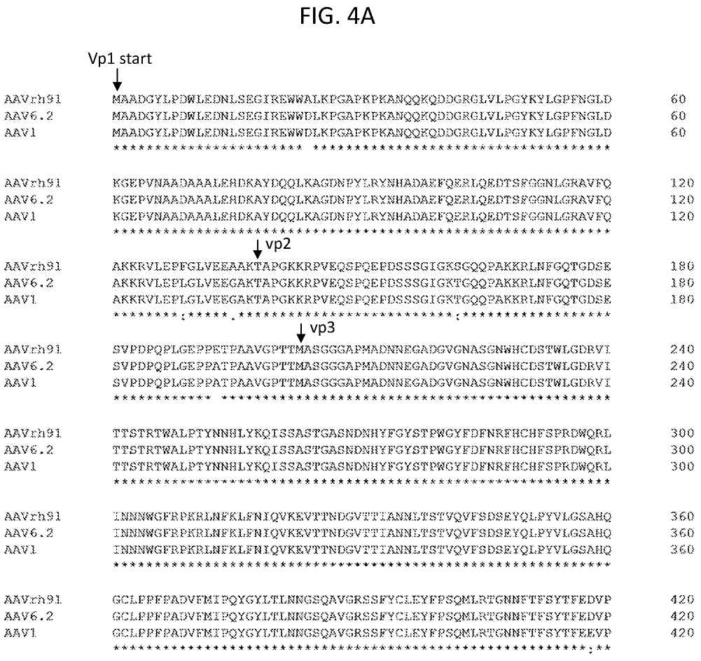

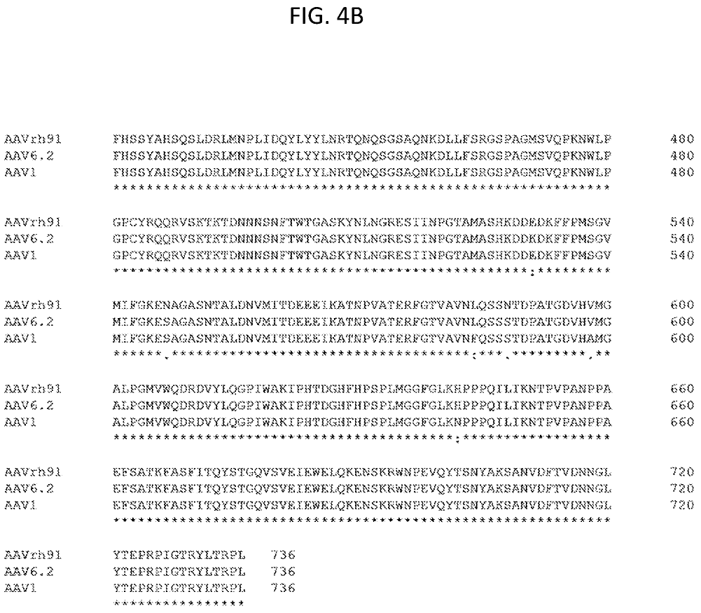

An alignment of the amino acid sequences for the capsids described herein is shown in FIG. 4A-FIG. 4B.

Included herein are rAAV comprising at least one of the vp1, vp2 and the vp3 of AAVrh91 (SEQ ID NO: 2). Also provided herein are rAAV comprising AAV capsids encoded by at least one of the vp1, vp2 and the vp3 of AAVrh91 (SEQ ID NO: 1) or AAVrh91eng (SEQ ID NO: 3).

In one embodiment, a composition is provided which includes a mixed population of recombinant adeno-associated virus (rAAV), each of said rAAV comprising: (a) an AAV capsid comprising about 60 capsid proteins made up of vp1 proteins, vp2 proteins and vp3 proteins, wherein the vp1, vp2 and vp3 proteins are: a heterogeneous population of vp1 proteins which are produced from a nucleic acid sequence encoding a selected AAV vp1 amino acid sequence, a heterogeneous population of vp2 proteins which are produced from a nucleic acid sequence encoding a selected AAV vp2 amino acid sequence, a heterogeneous population of vp3 proteins which produced from a nucleic acid sequence encoding a selected AAV vp3 amino acid sequence, wherein: the vp1, vp2 and vp3 proteins contain subpopulations with amino acid modifications comprising at least two highly deamidated asparagines (N) in asparagine-glycine pairs in the AAV capsid and optionally further comprising subpopulations comprising other deamidated amino acids, wherein the deamidation results in an amino acid change; and (b) a vector genome in the AAV capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, the deamidated asparagines are deamidated to aspartic acid, isoaspartic acid, an interconverting aspartic acid/isoaspartic acid pair, or combinations thereof. In certain embodiments, the capsid further comprises deamidated glutamine(s) which are deamidated to (α)-glutamic acid, γ-glutamic acid, an interconverting (α)-glutamic acid/γ-glutamic acid pair, or combinations thereof.

In certain embodiments, a novel isolated AAVrh91 capsid is provided. A nucleic acid sequence encoding the AAVrh91 capsid is provided in SEQ ID NO: 1 and the encoded amino acid sequence is provided in SEQ ID NO: 2. Provided herein is an rAAV comprising at least one of the vp1, vp2 and the vp3 of AAVrh91 (SEQ ID NO: 2). Also provided herein are rAAV comprising an AAV capsid encoded by at least one of the vp1, vp2 and the vp3 of AAVrh91 (SEQ ID NO: 1). In yet another embodiment, a nucleic acid sequence encoding the AAVrh91 amino acid sequence is provided in SEQ ID NO: 3 and the encoded amino acid sequence is provided in SEQ ID NO: 2. Also provided herein are rAAV comprising an AAV capsid encoded by at least one of the vp1, vp2 and the vp3 of AAVrh91eng (SEQ ID NO: 3). In certain embodiments, the vp1, vp2 and/or vp3 is the full-length capsid protein of AAVrh91 (SEQ ID NO: 2). In other embodiments, the vp1, vp2 and/or vp3 has an N-terminal and/or a C-terminal truncation (e.g. truncation(s) of about 1 to about 10 amino acids).

In a further aspect, a recombinant adeno-associated virus (rAAV) is provided which comprises: (A) an AAVrh91 capsid comprising one or more of: (1) AAVrh91 capsid proteins comprising: a heterogeneous population of AAVrh91 vp1 proteins selected from: vp1 proteins produced by expression from a nucleic acid sequence which encodes the predicted amino acid sequence of 1 to 736 of SEQ ID NO: 2, vp1 proteins produced from SEQ ID NO: 1, or vp1 proteins produced from a nucleic acid sequence at least 70% identical to SEQ ID NO: 1 which encodes the predicted amino acid sequence of 1 to 736 of SEQ ID NO: 2, a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 2, vp2 proteins produced from a sequence comprising at least nucleotides 412 to 2208 of SEQ ID NO: 1, or vp2 proteins produced from a nucleic acid sequence at least 70% identical to at least nucleotides 412 to 2208 of SEQ ID NO: 1 which encodes the predicted amino acid sequence of at least about amino acids 138 to 736 of SEQ ID NO: 2, a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 2, vp3 proteins produced from a sequence comprising at least nucleotides 607 to 2208 of SEQ ID NO: 1, or vp3 proteins produced from a nucleic acid sequence at least 70% identical to at least nucleotides 607 to 2208 of SEQ ID NO: 1 which encodes the predicted amino acid sequence of at least about amino acids 203 to 736 of SEQ ID NO: 1; and/or (2) a heterogeneous population of vp1 proteins which are the product of a nucleic acid sequence encoding the amino acid sequence of SEQ ID NO: 2, a heterogeneous population of vp2 proteins which are the product of a nucleic acid sequence encoding the amino acid sequence of at least about amino acids 138 to 736 of SEQ ID NO: 2, and a heterogeneous population of vp3 proteins which are the product of a nucleic acid sequence encoding at least amino acids 203 to 736 of SEQ ID NO: 2, wherein: the vp1, vp2 and vp3 proteins contain subpopulations with amino acid modifications comprising at least two highly deamidated asparagines (N) in asparagine-glycine pairs in SEQ ID NO: 2 and optionally further comprising subpopulations comprising other deamidated amino acids, wherein the deamidation results in an amino acid change; and (B) a vector genome in the AAVrh91 capsid, the vector genome comprising a nucleic acid molecule comprising AAV inverted terminal repeat sequences and a non-AAV nucleic acid sequence encoding a product operably linked to sequences which direct expression of the product in a host cell.

In yet another aspect, a recombinant adeno-associated virus (rAAV) is provided which comprises: (A) an AAVrh91 capsid comprising one or more of: (1) AAVrh91 capsid proteins comprising: a heterogeneous population of AAVrh91 vp1 proteins selected from: vp1 proteins produced by expression from a nucleic acid sequence which encodes the predicted amino acid sequence of 1 to 736 of SEQ ID NO: 2, vp1 proteins produced from SEQ ID NO: 3, or vp1 proteins produced from a nucleic acid sequence at least 70% identical to SEQ ID NO: 3 which encodes the predicted amino acid sequence of 1 to 736 of SEQ ID NO: 2, a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 2, vp2 proteins produced from a sequence comprising at least nucleotides 412 to 2208 of SEQ ID NO: 3, or vp2 proteins produced from a nucleic acid sequence at least 70% identical to at least nucleotides 412 to 2208 of SEQ ID NO: 3 which encodes the predicted amino acid sequence of at least about amino acids 138 to 736 of SEQ ID NO: 2, a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 2, vp3 proteins produced from a sequence comprising at least nucleotides 607 to 2208 of SEQ ID NO: 3, or vp3 proteins produced from a nucleic acid sequence at least 70% identical to at least nucleotides 607 to 2208 of SEQ ID NO: 3 which encodes the predicted amino acid sequence of at least about amino acids 203 to 736 of SEQ ID NO: 2; and/or (2) a heterogeneous population of vp1 proteins which are the product of a nucleic acid sequence encoding the amino acid sequence of SEQ ID NO: 2, a heterogeneous population of vp2 proteins which are the product of a nucleic acid sequence encoding the amino acid sequence of at least about amino acids 138 to 736 of SEQ ID NO: 2, and a heterogeneous population of vp3 proteins which are the product of a nucleic acid sequence encoding at least amino acids 203 to 736 of SEQ ID NO: 2, wherein: the vp1, vp2 and vp3 proteins contain subpopulations with amino acid modifications comprising at least two highly deamidated asparagines (N) in asparagine-glycine pairs in SEQ ID NO: 2 and optionally further comprising subpopulations comprising other deamidated amino acids, wherein the deamidation results in an amino acid change; and (B) a vector genome in the AAVrh91 capsid, the vector genome comprising a nucleic acid molecule comprising AAV inverted terminal repeat sequences and a non-AAV nucleic acid sequence encoding a product operably linked to sequences which direct expression of the product in a host cell.

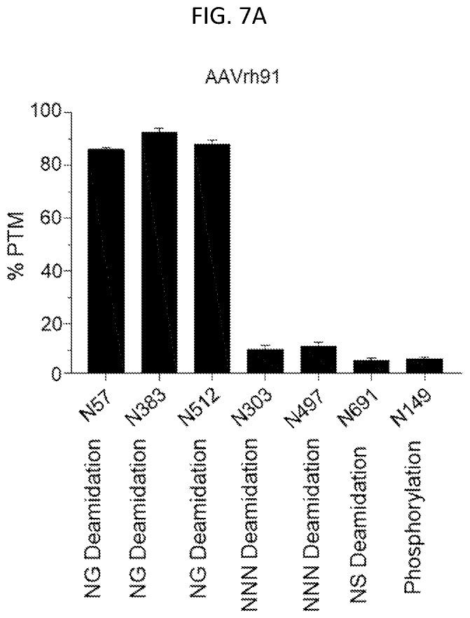

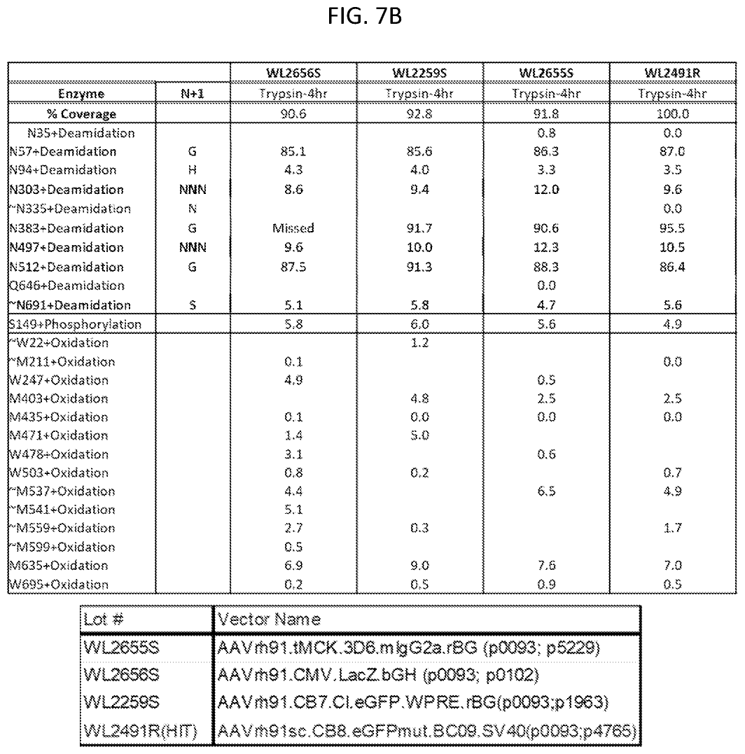

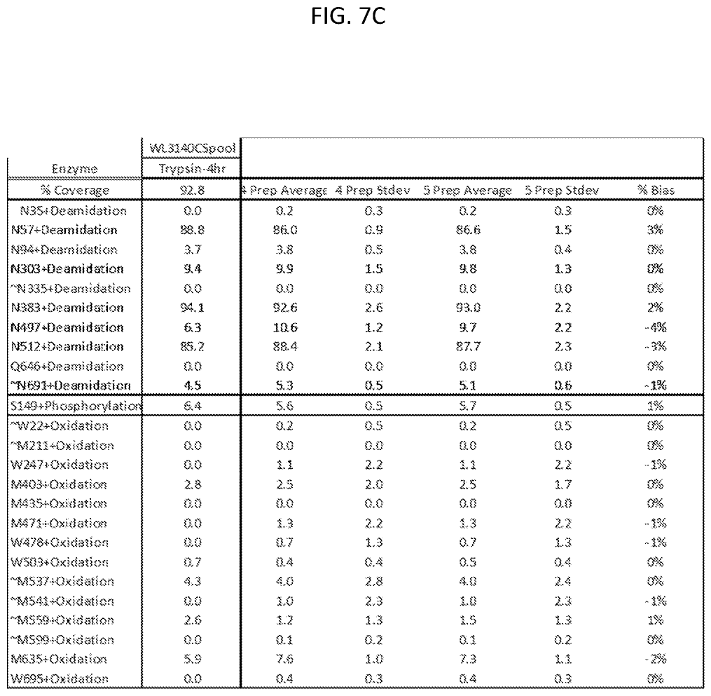

In certain embodiments, the AAVrh91 vp1, vp2 and vp3 proteins contain subpopulations with amino acid modifications comprising at least two highly deamidated asparagines (N) in asparagine-glycine pairs in SEQ ID NO: 2 and optionally further comprising subpopulations comprising other deamidated amino acids, wherein the deamidation results in an amino acid change. High levels of deamidation at N-G pairs N57, N383 and/or N512 are observed, relative to the number of SEQ ID NO: 2. Deamidation has been observed in other residues, as shown in the table below and in FIG. 7B and FIG. 7C. In certain embodiments, AAVrh91 may have other residues deamidated, e.g., typically at less than 10% and/or may have other modifications, including phosphorylation (e.g., where present, in the range of about 2 to about 30%, or about 2 to about 20%, or about 2 to about 10%) (e.g., at S149), or oxidation (e.g, at one or more of ˜W22, ˜M211, W247, M403, M435, M471, W478, W503, ˜M537, ˜M541, ˜M559, ˜M599, M635, and/or, W695). Optionally the W may oxidize to kynurenine.

TABLE
AAVrh91 Deamidation

| AAVrh91 | |
|---|---|
| Deamidation based | |
| on VP1 numbering | % Deamidation |
| N57 + Deamidation | 65-90, 70-95, 80-95, 75-100, 80-100, or 90-100 |
| N94 + Deamidation | 2-15 or 2-5 |
| N303 + Deamidation | 2-15 or 5-10 |
| N383 + Deamidation | 65-90, 70-95, 80-95, 75-100, 80-100, or 90-100 |
| N497 + Deamidation | 2-15 or 5-10 |
| N512 + Deamidation | 65-90, 70-95, 80-95, 75-100, 80-100, or 90-100 |
| ~N691 + Deamidation | 2-15, 2-10, or 5-10 |

In certain embodiments, an AAVrh91 capsid is modified in one or more of the positions identified in the preceding table, in the ranges provided, as determined using mass spectrometry with a trypsin enzyme. In certain embodiments, one or more of the positions, or the glycine following the N is modified as described herein. Residue numbers are based on the AAVrh91 sequence provided herein. See, SEQ ID NO: 2.

In certain embodiments, an AAVrh91 capsid comprises: a heterogeneous population of vp1 proteins which are the product of a nucleic acid sequence encoding the amino acid sequence of SEQ ID NO: 2, a heterogeneous population of vp2 proteins which are the product of a nucleic acid sequence encoding the amino acid sequence of at least about amino acids 138 to 736 of SEQ ID NO: 2, and a heterogeneous population of vp3 proteins which are the product of a nucleic acid sequence encoding at least amino acids 203 to 736 of SEQ ID NO: 2.

In certain embodiments, the nucleic acid sequence encoding the AAVrh91 vp1 capsid protein is provided in SEQ ID NO: 1. In other embodiments, a nucleic acid sequence of 70% to 99.9% identity to SEQ ID NO: 1, or 100% identical to SEQ ID NO: 1, may be selected to express the AAVrh91 capsid proteins. In certain other embodiments, the nucleic acid sequence is at least about 75% identical, at least 80% identical, at least 85%, at least 90%, at least 95%, at least 97% identical, at least 99%, or 100% identical to SEQ ID NO: 1. However, other nucleic acid sequences which encode the amino acid sequence of SEQ ID NO: 2 may be selected for use in producing rAAV capsids. In certain embodiments, the nucleic acid sequence has the nucleic acid sequence of SEQ ID NO: 1 or a sequence at least 70% to 99.9% identical, at least 75%, at least 80%, at least 85%, at least 90%, at least 95%, at least 97%, at least 99%, or 100% identical to SEQ ID NO: 1 which encodes SEQ ID NO: 2. In certain embodiments, the nucleic acid sequence has the nucleic acid sequence of SEQ ID NO: 1 or a sequence at least 70% to 99.9%, at least 75%, at least 80%, at least 85%, at least 90%, at least 95%, at least 97%, at least 99%, or 100% identical to about nt 412 to about nt 2208 of SEQ ID NO: 1 which encodes the vp2 capsid protein (about aa 138 to 736) of SEQ ID NO: 2. In certain embodiments, the nucleic acid sequence has the nucleic acid sequence of about nt 607 to about nt 2208 of SEQ ID NO: 1 or a sequence at least 70% to 99.9%, at least 75%, at least 80%, at least 85%, at least 90%, at least 95%, at least 97%, at least 99%, or 100% identical to nt 607 to about nt 2208 SEQ ID NO: 1 which encodes the vp3 capsid protein (about aa 203 to 736) of SEQ ID NO: 2.

In certain embodiments, the nucleic acid sequence encoding the AAVrh91 vp1 capsid protein is provided in SEQ ID NO: 3. In other embodiments, a nucleic acid sequence of 70% to 99.9% identity to SEQ ID NO: 3, or 100% identical to SEQ ID NO: 3, may be selected to express the AAVrh91 capsid proteins. In certain other embodiments, the nucleic acid sequence is at least about 75% identical, at least 80% identical, at least 85%, at least 90%, at least 95%, at least 97% identical, at least 99% to 99.9% identical, or 100% identical to SEQ ID NO: 3. However, other nucleic acid sequences which encode the amino acid sequence of SEQ ID NO: 2 may be selected for use in producing rAAV capsids. In certain embodiments, the nucleic acid sequence has the nucleic acid sequence of SEQ ID NO: 3 or a sequence at least 70% to 99.9% identical, at least 75%, at least 80%, at least 85%, at least 90%, at least 95%, at least 97%, at least 99%, or 100% identical to SEQ ID NO: 3 which encodes SEQ ID NO: 2. In certain embodiments, the nucleic acid sequence has the nucleic acid sequence of SEQ ID NO: 3 or a sequence at least 70% to 99.9%, at least 75%, at least 80%, at least 85%, at least 90%, at least 95%, at least 97%, at least 99%, or 100% identical to about nt 412 to about nt 2208 of SEQ ID NO: 3 which encodes the vp2 capsid protein (about aa 138 to 736) of SEQ ID NO: 2. In certain embodiments, the nucleic acid sequence has the nucleic acid sequence of about nt 607 to about nt 2208 of SEQ ID NO: 3 or a sequence at least 70% to 99.9%, at least 75%, at least 80%, at least 85%, at least 90%, at least 95%, at least 97%, at least 99% identical, or 100% identical to nt 607 to about nt 2208 SEQ ID NO: 3 which encodes the vp3 capsid protein (about aa 203 to 736) of SEQ ID NO: 2.

The invention also encompasses nucleic acid sequences encoding the AAVrh91 capsid sequence (SEQ ID NO: 2) or a mutant AAVrh91, in which one or more residues has been altered in order to decrease deamidation, or other modifications which are identified herein. Such nucleic acid sequences can be used in production of mutant AAVrh91 capsids.

In certain embodiments, provided herein is a nucleic acid molecule having the sequence of SEQ ID NO: 1 or a sequence at least 70%, at least 75%, at least 80%, at least 85%, at least 90%, at least 95%, at least 97%, at least 99%, or 100% identical to SEQ ID NO: 1 which encodes the vp1 amino acid sequence of SEQ ID NO: 2 with a modification (e.g., deamidated amino acid) as described herein. In certain embodiments, provided herein is a nucleic acid molecule having the sequence of SEQ ID NO: 3 or a sequence at least 70%, at least 75%, at least 80%, at least 85%, at least 90%, at least 95%, at least 97%, at least 99%, or 100% identical to SEQ ID NO: 3 which encodes the vp1 amino acid sequence of SEQ ID NO: 2 with a modification (e.g., deamidated amino acid) as described herein. In certain embodiments, the vp1 amino acid sequence is reproduced in SEQ ID NO: 2. In certain embodiments, a plasmid having a nucleic acid sequence described herein is provided. Such plasmids include a nucleic acid sequence that encodes at least one of the vp1, vp2, and vp3 of AAVrh91 (SEQ ID NO: 1), or a sequence sharing at least 95%, at least 96%, at least 97%, at least 98%, or at least 99% identity with a vp1, vp2, and/or vp3 sequence of SEQ ID NO: 1. In further embodiments, the plasmids include a non-AAV sequence. In certain embodiments, the plasmid comprises a WPRE and/or bGH-polyA signal. Cultured host cells containing the plasmids described herein are also provided.

Also provided herein are AAV capsid proteins that have been modified by introducing one or amino acid substitutions to, for example, improve capsid function/gene delivery to a target cell and/or improve the manufacture of an AAV vector by increasing yield. As described, differences between capsids a can result in differences in vector packaging. For example, despite being only 1.1% different in VP1 protein sequence, it was discovered that, based on vector yield, AAVrh91 vectors packaged transgene at significantly higher levels than AAV6.2 based vectors. AAVrh91 also packages transgene at levels higher than AAV1.

As described, a comparison of capsid structure has led to the identification of the locations of residues that differ between AAVrh91 and AAV1 capsids. Six of the residues are located in the AAVrh91 vp3 protein—Asp418, Asn547, Leu584, Asn588, Val598, and His642. Modification of AAV vectors, including other clade A vectors, to include these amino acid substitutions or conservative amino acid substitutions relative to those identified in the AAVrh91 capsid can result in a novel capsid with improved properties, including altered tropism and improved yield. Thus, in certain embodiments, provided herein are AAV having capsid proteins that have been modified to include one or more amino substitutions selected from: Asp at position 418, Asn at position 547, Leu at position 584, Asn at position at 588, Val at position 598, and His at position 642. In certain embodiments, a capsid protein is modified to include a Leu at position 584 (e.g., from a Leu) to increase manufacturing yields for a vector. In certain embodiments, an Ala or Val substitution at position 598 improves manufacturing yields of a vector. In certain embodiments, a capsid protein is modified to include an amino acid substitution at one or more residues selected from positions 418, 547, 584, 588, 598, and 642. The amino acid substitution can be a conservative amino acid substitution, i.e. replacement of an amino acid residue in an AAV capsid protein with another residue that is expected to have similar properties as the substitution observed at position 418, 547, 584, 588, 598, and/or 642 in the AAVrh91 capsid protein. In certain embodiments, a capsid protein is modified to include a Leu at position 584 and an Asn at position 547. The numbering of the positions can be determined by aligning a capsid protein sequence with the AAVrh91 amino acid sequence (SEQ ID NO: 2) or the AAV1 amino acid sequence (SEQ ID NO: 8). In certain embodiments, the capsid protein that is modified is an AAV1 capsid protein. In a further embodiment, the capsid protein that is modified has a sequence at least 95% identical or at least 99% identical to SEQ ID NO: 8. In other embodiments, the capsid that is modified is a clade A AAV capsid protein. In a further embodiment, the capsid protein that is modified is a AAVhu48R3, AAVhu48, AAVhu44, AAV.VR-355, AAV.VR-195, AAV6, or AAV6.2 capsid protein.

As used herein, the “conservative amino acid replacement” or “conservative amino acid substitutions” refers to a change, replacement or substitution of an amino acid to a different amino acid with similar biochemical properties (e.g. charge, hydrophobicity and size), which is known by practitioners of the art. Also see, e.g. FRENCH et al. What is a conservative substitution? Journal of Molecular Evolution, March 1983, Volume 19, Issue 2, pp 171-175 and YAMPOLSKY et al. The Exchangeability of Amino Acids in Proteins, Genetics. 2005 August; 170(4): 1459-1472, each of which is incorporated herein by reference in its entirety.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Identity may be determined by preparing an alignment of the sequences and through the use of a variety of algorithms and/or computer programs known in the art or commercially available [e.g., BLAST, ExPASy; ClustalO; FASTA; using, e.g., Needleman-Wunsch algorithm, Smith-Waterman algorithm]. Alignments are performed using any of a variety of publicly or commercially available Multiple Sequence Alignment Programs. Sequence alignment programs are available for amino acid sequences, e.g., the “Clustal Omega” “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Multiple sequence alignment programs are also available for nucleic acid sequences. Examples of such programs include, “Clustal Omega”, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

### B. RAAV VECTORS AND COMPOSITIONS

In another aspect, described herein are molecules which utilize the AAV capsid sequences described herein, including fragments thereof, for production of viral vectors useful in delivery of a heterologous gene or other nucleic acid sequences to a target cell. In one embodiment, the vectors useful in compositions and methods described herein contain, at a minimum, a sequence encoding an AAV capsid as described herein, e.g., an AAVrh91 capsid, or a fragment thereof. In another embodiment, useful vectors contain, at a minimum, sequences encoding a selected AAV serotype rep protein, or a fragment thereof. Optionally, such vectors may contain both AAV cap and rep proteins. In vectors in which both AAV rep and cap are provided, the AAV rep and AAV cap sequences can both be of one serotype origin, e.g., all an AAVrh91 origin. Alternatively, vectors may be used in which the rep sequences are from an AAV which differs from the wild type AAV providing the cap sequences. In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector, such as AAV2/8 described in U.S. Pat. No. 7,282,199, which is incorporated by reference herein. Optionally, the vectors further contain a minigene comprising a selected transgene which is flanked by AAV 5′ ITR and AAV 3′ ITR. In another embodiment, the AAV is a self-complementary AAV (sc-AAV) (See, US 2012/0141422 which is incorporated herein by reference). Self-complementary vectors package an inverted repeat genome that can fold into dsDNA without the requirement for DNA synthesis or base-pairing between multiple vector genomes. Because scAAV have no need to convert the single-stranded DNA (ssDNA) genome into double-stranded DNA (dsDNA) prior to expression, they are more efficient vectors. However, the trade-off for this efficiency is the loss of half the coding capacity of the vector, ScAAV are useful for small protein-coding genes (up to ˜55 kd) and any currently available RNA-based therapy.

Pseudotyped vectors, wherein the capsid of one AAV is replaced with a heterologous capsid protein, are useful herein. For illustrative purposes, AAV vectors utilizing an AAVrh91 capsid as described herein, with AAV2 ITRs are used in the examples described below. See, Mussolino et al, cited above. Unless otherwise specified, the AAV ITRs, and other selected AAV components described herein, may be individually selected from among any AAV serotype, including, without limitation, AAV1, AAV2, AAV3, AAV4, AAV5, AAV6, AAV7, AAV8, AAV9 or other known and unknown AAV serotypes. In one desirable embodiment, the ITRs of AAV serotype 2 are used. However, ITRs from other suitable serotypes may be selected. These ITRs or other AAV components may be readily isolated using techniques available to those of skill in the art from an AAV serotype. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

The rAAV described herein also comprise a vector genome. The vector genome is composed of, at a minimum, a non-AAV or heterologous nucleic acid sequence (the transgene), as described below, and its regulatory sequences, and 5′ and 3′ AAV inverted terminal repeats (ITRs). It is this minigene which is packaged into a capsid protein and delivered to a selected target cell.

The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a target cell. The heterologous nucleic acid sequence (transgene) can be derived from any organism. The AAV may comprise one or more transgenes.

In certain embodiments, provided herein is a rAAVrh91 vector that includes a transgene comprising a sequence encoding erythropoietin (EPO). In certain embodiments, the transgene encodes a canine or feline EPO gene. Such recombinant vectors are suitable, for example, for use in a regimen for treating chronic kidney disease and other conditions in a subject characterized by a decrease in the amount of circulating red blood cells.

In certain embodiments, provided herein is a rAAVrh91 vector that includes a transgene comprising a sequence encoding an anti-nerve growth factor (NGF) antibody. In certain embodiments, the transgene encodes a canine or feline anti-NGF antibody. Such recombinant vectors are suitable, for example, for use in a regimen for treating osteoarthritis pain in a subject.

In certain embodiments, provided herein is a rAAVrh91 vector that includes a transgene comprising a sequence encoding glucagon-like peptide 1 (GLP-1). In certain embodiments, the transgene encodes a canine or feline GLP-1. Such recombinant vectors are suitable, for example, for use in a regimen for treating type II diabetes in a subject.

In certain embodiments, provided herein is a rAAVrh91 vector that includes a transgene comprising a sequence encoding insulin. In certain embodiments, the transgene encodes a canine or feline insulin. Such recombinant vectors are suitable, for example, for use in a regimen for treating type I diabetes or type II diabetes in a subject.

In certain embodiments, provided herein is a rAAVrh91 vector that includes a transgene comprising a CLN (ceroid lipofuscinosis, neuronal) gene. In certain embodiments, the transgene encodes palmitoyl-protein thioesterase 1—PPT1 (CLN1). In certain embodiments, the transgene encodes tripeptidyl peptidase 1—TPP1 (CLN2). In certain embodiments, the transgene encodes CLN3 (CLN3). In certain embodiments, the transgene encodes DNAJC5 (CLN4). In certain embodiments, the transgene encodes CLN5 (CLN5). In certain embodiments, the transgene encodes CLN6 (CLN6). In certain embodiments, the transgene encodes MFSD8 (CLN7). In certain embodiments, the transgene encodes CLN8 (CLN8). In certain embodiments, the transgene encodes CTSD (CLN10). In certain embodiments, the transgene encodes GRN (CLN11). In certain embodiments, the transgene encodes ATP13A2 (CLN12). In certain embodiments, the transgene encodes ATP13A2 (CLN13). Such recombinant vectors are suitable, for example, for use in a regimen for treating Batten disease or Neuronal Ceroid Lipofuscinosis (NCL) in a subject.

In certain embodiments, provided herein is a rAAVrh91 vector that includes a transgene comprising a sequence encoding PTEN-induced kinase 1, a mitochondrial serine/threonine-protein kinase encoded by the PINK1 gene. Such recombinant vectors are suitable, for example, for use in a regimen for treating young-onset Parkinson disease.

In certain embodiments, provided herein is a rAAVrh91 vector that includes a transgene comprising a sequence encoding an antagonist for IgE, IL-32, or the interleukin-4 receptor alpha (IL-4Rα) subunit of IL-4/IL-13 receptors, including, e.g., antibodies and receptor-IgG fusion proteins. In certain embodiments, the transgene encodes an antagonist for a canine or feline IgE, IL-32, or IL-4Rα subunit. Such recombinant vectors are suitable, for example, for use in a regimen for treating atopic dermatitis in a subject.

The composition of the transgene sequence will depend upon the use to which the resulting vector will be put. For example, one type of transgene sequence includes a reporter sequence, which upon expression produces a detectable signal. Such reporter sequences include, without limitation, DNA sequences encoding s-lactamase, β-galactosidase (LacZ), alkaline phosphatase, thymidine kinase, green fluorescent protein (GFP), enhanced GFP (EGFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

However, desirably, the transgene is a non-marker sequence encoding a product which is useful in biology and medicine, such as proteins, peptides, RNA, enzymes, dominant negative mutants, or catalytic RNAs. Desirable RNA molecules include tRNA, dsRNA, ribosomal RNA, catalytic RNAs, siRNA, small hairpin RNA, trans-splicing RNA, and antisense RNAs. One example of a useful RNA sequence is a sequence which inhibits or extinguishes expression of a targeted nucleic acid sequence in the treated animal. Typically, suitable target sequences include oncologic targets and viral diseases. See, for examples of such targets the oncologic targets and viruses identified below in the section relating to immunogens.

The transgene may be used to correct or ameliorate gene deficiencies, which may include deficiencies in which normal genes are expressed at less than normal levels or deficiencies in which the functional gene product is not expressed. Alternatively, the transgene may provide a product to a cell which is not natively expressed in the cell type or in the host. A preferred type of transgene sequence encodes a therapeutic protein or polypeptide which is expressed in a host cell. The invention further includes using multiple transgenes. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In order for the cell to produce the multi-subunit protein, a cell is infected with the recombinant virus containing each of the different subunits. Alternatively, different subunits of a protein may be encoded by the same transgene. In this case, a single transgene includes the DNA encoding each of the subunits, with the DNA for each subunit separated by an internal ribozyme entry site (IRES). This is desirable when the size of the DNA encoding each of the subunits is small, e.g., the total size of the DNA encoding the subunits and the IRES is less than five kilobases. As an alternative to an IRES, the DNA may be separated by sequences encoding a 2A peptide, which self-cleaves in a post-translational event. See, e.g., M. L. Donnelly, et al, J. Gen. Virol., 78(Pt 1):13-21 (January 1997); Furler, S., et al, Gene Ther., 8(11):864-873 (June 2001); Klump H., et al., Gene Ther., 8(10):811-817 (May 2001). This 2A peptide is significantly smaller than an IRES, making it well suited for use when space is a limiting factor. More often, when the transgene is large, consists of multi-subunits, or two transgenes are co-delivered, rAAV carrying the desired transgene(s) or subunits are co-administered to allow them to concatamerize in vivo to form a single vector genome. In such an embodiment, a first AAV may carry an expression cassette which expresses a single transgene and a second AAV may carry an expression cassette which expresses a different transgene for co-expression in the host cell. However, the selected transgene may encode any biologically active product or other product, e.g., a product desirable for study.

Examples of suitable transgenes or gene products include those associated with familial hypercholesterolemia, muscular dystrophy, cystic fibrosis, and rare or orphan diseases. Examples of such rare disease may include spinal muscular atrophy (SMA), Huntingdon's Disease, Rett Syndrome (e.g., methyl-CpG-binding protein 2 (MeCP2); UniProtKB—P51608), Amyotrophic Lateral Sclerosis (ALS), Duchenne Type Muscular dystrophy, Friedrichs Ataxia (e.g., frataxin), ATXN2 associated with spinocerebellar ataxia type 2 (SCA2)/ALS; TDP-43 associated with ALS, progranulin (PRGN) (associated with non-Alzheimer's cerebral degenerations, including, frontotemporal dementia (FTD), progressive non-fluent aphasia (PNFA) and semantic dementia), CDKL5 deficiency, Angelman syndrome, N-glycanase 1 deficiency, Alzheimer's disease, Fragile X syndrome, Neimann Pick disease (including types A and B (ASMD or Acid Sphingomyelinase Deficiency), and type c (NPC), mucopolysaccharidoses (MPS), Wolman disease, Tay-Sachs disease, among others. See, e.g., www.orpha.net/consor/cgi-bin/Disease_Search_List.php; rarediseases.info.nih.gov/diseases.

Useful therapeutic products encoded by the transgene include hormones and growth and differentiation factors including, without limitation, insulin, glucagon, glucagon-like peptide 1 (GLP-1), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), any one of the transforming growth factor β superfamily, including TGF β, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), lysosomal acid lipase (LIPA or LAL), neurturin, agrin, any one of the family of semaphorins/collapsins, netrin-1 and netrin-2, hepatocyte growth factor (HGF), ephrins, noggin, sonic hedgehog and tyrosine hydroxylase. Other useful transgene encode lysosomal enzymes that cause mucopolysaccharidoses (MPS), including α-L-iduronidase (MPSI), iduronate sulfatase (MPSII), heparan N-sulfatase (sulfaminidase) (MPS IIIA, Sanfilippo A), α-N-acetyl-glucosaminidase (MPS IIIB, Sanfilippo B), acetyl-CoA:α-glucosaminide acetyltransferase (MPS IIIC, Sanfilippo C), N-acetylglucosamine 6-sulfatase (MPS HID, Sanfilippo D), galactose 6-sulfatase (MPS IVA, Morquio A), β-Galactosidase (MPS IVB, Morquio B), N-acetyl-galactosamine 4-sulfatase (MPS VI, Maroteaux-Lamy), (β-Glucuronidase (MPS VII, Sly), and hyaluronidase (MPS IX).

Other useful transgene products include proteins that regulate the immune system including, without limitation, cytokines and lymphokines such as thrombopoietin (TPO), interleukins (IL) IL-1 through IL-25 (including, IL-2, IL-4, IL-12, and IL-18), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

Still other useful gene products include any one of the receptors for the hormones, growth factors, cytokines, lymphokines, regulatory proteins and immune system proteins. The invention encompasses receptors for cholesterol regulation, including the low density lipoprotein (LDL) receptor, high density lipoprotein (HDL) receptor, the very low density lipoprotein (VLDL) receptor, and the scavenger receptor. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin sequence or functional fragment thereof. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encodes β-glucuronidase (GUSB)). In another example, the gene product is ubiquitin protein ligase E3A (UBE3A). Still useful gene products include UDP Glucuronosyltransferase Family 1 Member A1 (UGT1A1).

Other useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce self-directed antibodies. T cell mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

Still other useful gene products include those used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349). In some embodiments, the minigene comprises first 57 base pairs of the Factor VIII heavy chain which encodes the 10 amino acid signal sequence, as well as the human growth hormone (hGH) polyadenylation sequence. In alternative embodiments, the minigene further comprises the A1 and A2 domains, as well as 5 amino acids from the N-terminus of the B domain, and/or 85 amino acids of the C-terminus of the B domain, as well as the A3, C1 and C2 domains. In yet other embodiments, the nucleic acids encoding Factor VIII heavy chain and light chain are provided in a single minigene separated by 42 nucleic acids coding for 14 amino acids of the B domain [U.S. Pat. No. 6,200,560].

Further illustrative genes which may be delivered via the rAAV include, without limitation, glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; (NGLY1) N-glycanase 1; galactose-1 phosphate uridyl transferase, associated with galactosemia; phenylalanine hydroxylase (PAH), associated with phenylketonuria (PKU); gene products associated with Primary Hyperoxaluria Type 1 including Hydroxyacid Oxidase 1 (GO/HAO1) and AGXT, branched chain alpha-ketoacid dehydrogenase, including BCKDH, BCKDH-E2, BAKDH-E1a, and BAKDH-E1b, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type C1); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO 2015/164778; PCSK9; ApoE and ApoC proteins, associated with dementia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccsinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

In certain embodiments, the rAAV may be used in gene editing systems, which system may involve one rAAV or co-administration of multiple rAAV stocks. For example, the rAAV may be engineered to deliver SpCas9, SaCas9, ARCUS, Cpf1 (also known as Cas12a), CjCas9, and other suitable gene editing constructs.

In certain embodiments, a rAAV-based gene editing nuclease system is provided herein. The gene editing nuclease targets sites in a disease-associated gene, i.e., gene of interest.

In certain embodiments, the AAV-based gene editing nuclease system comprises an rAAV comprising an AAVrh91 capsid and enclosed therein a vector genome, wherein the vector genome comprising AAV 5′ inverted terminal repeats (ITR), an expression cassette comprising a nucleic acid sequence encoding a gene editing nuclease which recognizes and cleaves a recognition site in a gene of interest, wherein said gene editing nuclease coding sequence is operably linked to expression control sequences which direct expression thereof in a cell comprising the gene of interest, and an AAV 3′ ITR. Provided herein also is a method of treatment using an rAAV-based gene editing nuclease system.

In some embodiments, the rAAV-based gene editing meganuclease system is used for treating diseases, disorders, syndrome and/or conditions. In some embodiments, the gene editing nuclease is targeted to a gene of interest, wherein the gene of interest has one or more genetic mutation, deletion, insertion, and/or a defect which is associated with and/or implicated in a disease, disorder, syndrome and/or conditions. In some embodiments, the disorder is selected but not limited to cardiovascular, hepatic, endocrine or metabolic, musculoskeletal, neurological, and/or renal disorders.

Alternatively, or in addition, the vectors of the invention may contain AAV sequences of the invention and a transgene encoding a peptide, polypeptide or protein which induces an immune response to a selected immunogen. For example, immunogens may be selected from a variety of viral families. Example of desirable viral families against which an immune response would be desirable include, the picomavirus family, which includes the genera rhinoviruses, which are responsible for about 50% of cases of the common cold; the genera enteroviruses, which include polioviruses, coxsackieviruses, echoviruses, and human enteroviruses such as hepatitis A virus; and the genera apthoviruses, which are responsible for foot and mouth diseases, primarily in non-human animals. Within the picornavirus family of viruses, target antigens include the VP1, VP2, VP3, VP4, and VPG. Another viral family includes the calcivirus family, which encompasses the Norwalk group of viruses, which are an important causative agent of epidemic gastroenteritis. Still another viral family desirable for use in targeting antigens for inducing immune responses in humans and non-human animals is the togavirus family, which includes the genera alphavirus, which include Sindbis viruses, RossRiver virus, and Venezuelan, Eastern & Western Equine encephalitis, and rubivirus, including Rubella virus. The flaviviridae family includes dengue, yellow fever, Japanese encephalitis, St. Louis encephalitis and tick borne encephalitis viruses. Other target antigens may be generated from the Hepatitis C or the coronavirus family, which includes a number of non-human viruses such as infectious bronchitis virus (poultry), porcine transmissible gastroenteric virus (pig), porcine hemagglutinating encephalomyelitis virus (pig), feline infectious peritonitis virus (cats), feline enteric coronavirus (cat), canine coronavirus (dog), and human respiratory coronaviruses, which may cause the common cold and/or non-A, B or C hepatitis. Within the coronavirus family, target antigens include the E1 (also called M or matrix protein), E2 (also called S or Spike protein), E3 (also called HE or hemagglutin-elterose) glycoprotein (not present in all coronaviruses), or N (nucleocapsid). Still other antigens may be targeted against the rhabdovirus family, which includes the genera vesiculovirus (e.g., Vesicular Stomatitis Virus), and the general lyssavirus (e.g., rabies). Within the rhabdovirus family, suitable antigens may be derived from the G protein or the N protein. The family filoviridae, which includes hemorrhagic fever viruses such as Marburg and Ebola virus may be a suitable source of antigens. The paramyxovirus family includes parainfluenza Virus Type 1, parainfluenza Virus Type 3, bovine parainfluenza Virus Type 3, rubulavirus (mumps virus, parainfluenza Virus Type 2, parainfluenza virus Type 4, Newcastle disease virus (chickens), rinderpest, morbillivirus, which includes measles and canine distemper, and pneumovirus, which includes respiratory syncytial virus. The influenza virus is classified within the family orthomyxovirus and is a suitable source of antigen (e.g., the HA protein, the N1 protein). The bunyavirus family includes the genera bunyavirus (California encephalitis, La Crosse), phlebovirus (Rift Valley Fever), hantavirus (puremala is a hemahagin fever virus), nairovirus (Nairobi sheep disease) and various unassigned bungaviruses. The arenavirus family provides a source of antigens against LCM and Lassa fever virus. The reovirus family includes the genera reovirus, rotavirus (which causes acute gastroenteritis in children), orbiviruses, and cultivirus (Colorado Tick fever, Lebombo (humans), equine encephalosis, blue tongue).

The retrovirus family includes the sub-family oncorivirinal which encompasses such human and veterinary diseases as feline leukemia virus, HTLVI and HTLVII, lentivirinal (which includes human immunodeficiency virus (HIV), simian immunodeficiency virus (SIV), feline immunodeficiency virus (FIV), equine infectious anemia virus, and spumavirinal). Between the HIV and SIV, many suitable antigens have been described and can readily be selected. Examples of suitable HIV and SIV antigens include, without limitation the gag, pol, Vif, Vpx, VPR, Env, Tat and Rev proteins, as well as various fragments thereof. In addition, a variety of modifications to these antigens have been described. Suitable antigens for this purpose are known to those of skill in the art. For example, one may select a sequence encoding the gag, pol, Vif, and Vpr, Env, Tat and Rev, amongst other proteins. See, e.g., the modified gag protein which is described in U.S. Pat. No. 5,972,596. See, also, the HIV and SIV proteins described in D. H. Barouch et al, J. Virol., 75(5):2462-2467 (March 2001), and R. R. Amara, et al, Science, 292:69-74 (6 Apr. 2001). These proteins or subunits thereof may be delivered alone, or in combination via separate vectors or from a single vector.

The papovavirus family includes the sub-family polyomaviruses (BKU and JCU viruses) and the sub-family papillomavirus (associated with cancers or malignant progression of papilloma). The adenovirus family includes viruses (EX, AD7, ARD, O.B.) which cause respiratory disease and/or enteritis. The parvovirus family feline parvovirus (feline enteritis), feline panleucopeniavirus, canine parvovirus, and porcine parvovirus. The herpesvirus family includes the sub-family alphaherpesvirinae, which encompasses the genera simplexvirus (HSVI, HSVII), varicellovirus (pseudorabies, varicella zoster) and the sub-family betaherpesvirinae, which includes the genera cytomegalovirus (HCMV, muromegalovirus) and the sub-family gammaherpesvirinae, which includes the genera lymphocryptovirus, EBV (Burkitts lymphoma), infectious rhinotracheitis, Marek's disease virus, and rhadinovirus. The poxvirus family includes the sub-family chordopoxvirinae, which encompasses the genera orthopoxvirus (Variola (Smallpox) and Vaccinia (Cowpox)), parapoxvirus, avipoxvirus, capripoxvirus, leporipoxvirus, suipoxvirus, and the sub-family entomopoxvirinae. The hepadnavirus family includes the Hepatitis B virus. One unclassified virus which may be suitable source of antigens is the Hepatitis delta virus. Still other viral sources may include avian infectious bursal disease virus and porcine respiratory and reproductive syndrome virus. The alphavirus family includes equine arteritis virus and various Encephalitis viruses.

The present invention may also encompass immunogens which are useful to immunize a human or non-human animal against other pathogens including bacteria, fungi, parasitic microorganisms or multicellular parasites which infect human and non-human vertebrates, or from a cancer cell or tumor cell. Examples of bacterial pathogens include pathogenic gram-positive cocci include pneumococci; staphylococci; and streptococci.

Pathogenic gram-negative cocci include meningococcus; gonococcus. Pathogenic enteric gram-negative bacilli include enterobacteriaceae; Pseudomonas, acinetobacteria and Eikenella; melioidosis; Salmonella; Shigella; Haemophilus; Moraxella; H. ducreyi (which causes chancroid); Brucella; Franisella tularensis (which causes tularemia); Yersinia (Pasteurella); Streptobacillus Moniliformis and Spirillum; Gram-Positive Bacilli Include Listeria monocytogenes; Erysipelothrix rhusiopathiae; Corynebacterium diphtheria (diphtheria); cholera; B. anthracis (anthrax); donovanosis (granuloma inguinale); and bartonellosis. Diseases caused by pathogenic anaerobic bacteria include tetanus; botulism; other clostridia; tuberculosis; leprosy; and other mycobacteria. Pathogenic spirochetal diseases include syphilis; treponematoses: yaws, pinta and endemic syphilis; and leptospirosis. Other infections caused by higher pathogen bacteria and pathogenic fungi include actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infections include Typhus fever, Rocky Mountain spotted fever, Q fever, and Rickettsialpox. Examples of Mycoplasma and chlamydial infections include: Mycoplasma pneumoniae; lymphogranuloma venereum; psittacosis; and perinatal chlamydial infections. Pathogenic eukaryotes encompass pathogenic protozoans and helminths and infections produced thereby include: amebiasis; malaria; leishmaniasis; trypanosomiasis; toxoplasmosis; Pneumocystis carinii; Trichans; Toxoplasma gondii; babesiosis; giardiasis; trichinosis; filariasis; schistosomiasis; nematodes; trematodes or flukes; and cestode (tapeworm) infections.

Many of these organisms and/or toxins produced thereby have been identified by the Centers for Disease Control [(CDC), Department of Health and Human Services, USA], as agents which have potential for use in biological attacks. For example, some of these biological agents, include, Bacillus anthracis (anthrax), Clostridium botulinum and its toxin (botulism), Yersinia pestis (plague), Variola major (smallpox), Francisella tularensis (tularemia), and viral hemorrhagic fever, all of which are currently classified as Category A agents; Coxiella burnetti (Q fever); Brucella species (brucellosis), Burkholderia mallei (glanders), Ricinus communis and its toxin (ricin toxin), Clostridium perfringens and its toxin (epsilon toxin), Staphylococcus species and their toxins (enterotoxin B), all of which are currently classified as Category B agents; and Nipan virus and hantaviruses, which are currently classified as Category C agents. In addition, other organisms, which are so classified or differently classified, may be identified and/or used for such a purpose in the future. It will be readily understood that the viral vectors and other constructs described herein are useful to deliver antigens from these organisms, viruses, their toxins or other by-products, which will prevent and/or treat infection or other adverse reactions with these biological agents.

Administration of the vectors of the invention to deliver immunogens against the variable region of the T cells elicit an immune response including CTLs to eliminate those T cells. In rheumatoid arthritis (RA), several specific variable regions of T cell receptors (TCRs) which are involved in the disease have been characterized. These TCRs include V-3, V-14, V-17 and Vα-17. Thus, delivery of a nucleic acid sequence that encodes at least one of these polypeptides will elicit an immune response that will target T cells involved in RA. In multiple sclerosis (MS), several specific variable regions of TCRs which are involved in the disease have been characterized. These TCRs include V-7 and Vα-10. Thus, delivery of a nucleic acid sequence that encodes at least one of these polypeptides will elicit an immune response that will target T cells involved in MS. In scleroderma, several specific variable regions of TCRs which are involved in the disease have been characterized. These TCRs include V-6, V-8, V-14 and Vα-16, Vα-3C, Vα-7, Vα-14, Vα-15, Vα-16, Vα-28 and Vα-12. Thus, delivery of a nucleic acid molecule that encodes at least one of these polypeptides will elicit an immune response that will target T cells involved in scleroderma.

In one embodiment, the transgene is selected to provide optogenetic therapy. In optogenetic therapy, artificial photoreceptors are constructed by gene delivery of light-activated channels or pumps to surviving cell types in the remaining retinal circuit. This is particularly useful for patients who have lost a significant amount of photoreceptor function, but whose bipolar cell circuitry to ganglion cells and optic nerve remains intact. In one embodiment, the heterologous nucleic acid sequence (transgene) is an opsin. The opsin sequence can be derived from any suitable single- or multicellular-organism, including human, algae and bacteria. In one embodiment, the opsin is rhodopsin, photopsin, L/M wavelength (red/green)-opsin, or short wavelength (S) opsin (blue). In another embodiment, the opsin is channelrhodopsin or halorhodopsin.

In another embodiment, the transgene is selected for use in gene augmentation therapy, i.e., to provide replacement copy of a gene that is missing or defective. In this embodiment, the transgene may be readily selected by one of skill in the art to provide the necessary replacement gene. In one embodiment, the missing/defective gene is related to an ocular disorder. In another embodiment, the transgene is NYX, GRM6, TRPM1L or GPR179 and the ocular disorder is Congenital Stationary Night Blindness. See, e.g., Zeitz et al, Am J Hum Genet. 2013 Jan. 10; 92(1):67-75. Epub 2012 Dec. 13 which is incorporated herein by reference. In another embodiment, the transgene is RPGR.

In another embodiment, the transgene is selected for use in gene suppression therapy, i.e., expression of one or more native genes is interrupted or suppressed at transcriptional or translational levels. This can be accomplished using short hairpin RNA (shRNA) or other techniques well known in the art. See, e.g., Sun et al, Int J Cancer. 2010 Feb. 1; 126(3):764-74 and O'Reilly M, et al. Am J Hum Genet. 2007 July; 81(1):127-35, which are incorporated herein by reference. In this embodiment, the transgene may be readily selected by one of skill in the art based upon the gene which is desired to be silenced.

In another embodiment, the transgene comprises more than one transgene. This may be accomplished using a single vector carrying two or more heterologous sequences, or using two or more AAV each carrying one or more heterologous sequences. In one embodiment, the AAV is used for gene suppression (or knockdown) and gene augmentation co-therapy. In knockdown/augmentation co-therapy, the defective copy of the gene of interest is silenced and a non-mutated copy is supplied. In one embodiment, this is accomplished using two or more co-administered vectors. See, Millington-Ward et al, Molecular Therapy, April 2011, 19(4):642-649 which is incorporated herein by reference. The transgenes may be readily selected by one of skill in the art based on the desired result.

In another embodiment, the transgene is selected for use in gene correction therapy. This may be accomplished using, e.g., a zinc-finger nuclease (ZFN)-induced DNA double-strand break in conjunction with an exogenous DNA donor substrate. See, e.g., Ellis et al, Gene Therapy (epub January 2012) 20:35-42 which is incorporated herein by reference. The transgenes may be readily selected by one of skill in the art based on the desired result.

In one embodiment, the capsids described herein are useful in the CRISPR-Cas dual vector system described in U.S. Provisional Patent Application Nos. 61/153,470, 62/183,825, 62/254,225 and 62/287,511, each of which is incorporated herein by reference. The capsids are also useful for delivery of homing endonucleases or other meganucleases.

In another embodiment, the transgenes useful herein include reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

Desirably, the transgene encodes a product which is useful in biology and medicine, such as proteins, peptides, RNA, enzymes, or catalytic RNAs. Desirable RNA molecules include shRNA, tRNA, dsRNA, ribosomal RNA, catalytic RNAs, and antisense RNAs. One example of a useful RNA sequence is a sequence which extinguishes expression of a targeted nucleic acid sequence in the treated animal.

The regulatory sequences include conventional control elements which are operably linked to the transgene in a manner which permits its transcription, translation and/or expression in a cell transfected with the vector or infected with the virus produced as described herein. As used herein, “operably linked” sequences include both expression control sequences that are contiguous with the gene of interest and expression control sequences that act in trans or at a distance to control the gene of interest.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters, are known in the art and may be utilized.

The regulatory sequences useful in the constructs provided herein may also contain an intron, desirably located between the promoter/enhancer sequence and the gene. One desirable intron sequence is derived from SV-40, and is a 100 bp mini-intron splice donor/splice acceptor referred to as SD-SA. Another suitable sequence includes the woodchuck hepatitis virus post-transcriptional element. (See, e.g., L. Wang and I. Verma, 1999 Proc. Natl. Acad. Sci., USA, 96:3906-3910). PolyA signals may be derived from many suitable species, including, without limitation SV-40, human and bovine.

Another regulatory component of the rAAV useful in the methods described herein is an internal ribosome entry site (IRES). An IRES sequence, or other suitable systems, may be used to produce more than one polypeptide from a single gene transcript. An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ to the transgene in the rAAV vector.

In one embodiment, the expression cassette or vector genome comprises a promoter (or a functional fragment of a promoter). The selection of the promoter to be employed in the rAAV may be made from among a wide number of constitutive or inducible promoters that can express the selected transgene in the desired target cell. In one embodiment, the target cell is an ocular cell. The promoter may be derived from any species, including human. Desirably, in one embodiment, the promoter is “cell specific”. The term “cell-specific” means that the particular promoter selected for the recombinant vector can direct expression of the selected transgene in a particular cell tissue. In one embodiment, the promoter is specific for expression of the transgene in muscle cells. In another embodiment, the promoter is specific for expression in lung. In another embodiment, the promoter is specific for expression of the transgene in liver cells. In another embodiment, the promoter is specific for expression of the transgene in airway epithelium. In another embodiment, the promoter is specific for expression of the transgene in neurons. In another embodiment, the promoter is specific for expression of the transgene in heart.

The expression cassette typically contains a promoter sequence as part of the expression control sequences, e.g., located between the selected 5′ ITR sequence and the immunoglobulin construct coding sequence. In one embodiment, expression in liver is desirable. Thus, in one embodiment, a liver-specific promoter is used. Tissue specific promoters, constitutive promoters, regulatable promoters [see, e.g., WO 2011/126808 and WO 2013/04943], or a promoter responsive to physiologic cues may be used may be utilized in the vectors described herein. In another embodiment, expression in muscle is desirable. Thus, in one embodiment, a muscle-specific promoter is used. In one embodiment, the promoter is an MCK based promoter, such as the dMCK (509-bp) or tMCK (720-bp) promoters (see, e.g., Wang et al, Gene Ther. 2008 November; 15(22):1489-99. doi: 10.1038/gt.2008.104. Epub 2008 Jun. 19, which is incorporated herein by reference). Another useful promoter is the SPc5-12 promoter (see Rasowo et al, European Scientific Journal June 2014 edition vol. 10, No. 18, which is incorporated herein by reference). In one embodiment, the promoter is a CMV promoter. In another embodiment, the promoter is a TBG promoter. In another embodiment, a CB7 promoter or CAG promoter is used. CB7 is a chicken β-actin promoter with cytomegalovirus enhancer elements. Alternatively, other liver-specific promoters may be used [see, e.g., The Liver Specific Gene Promoter Database, Cold Spring Harbor, rulai.schl.edu/LSPD, alpha 1 anti-trypsin (A1AT); human albumin Miyatake et al., J. Virol., 71:5124 32 (1997), humAlb; and hepatitis B virus core promoter, Sandig et al., Gene Ther., 3:1002 9 (1996)]. TTR minimal enhancer/promoter, alpha-antitrypsin promoter, LSP (845 nt)25(requires intron-less scAAV).

The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter.

The expression cassette may contain at least one enhancer, i.e., CMV enhancer. Still other enhancer elements may include, e.g., an apolipoprotein enhancer, a zebrafish enhancer, a GFAP enhancer element, and brain specific enhancers such as described in WO 2013/1555222, woodchuck post hepatitis post-transcriptional (WPRE) regulatory element. Additionally, or alternatively, other, e.g., the hybrid human cytomegalovirus (HCMV)-immediate early (IE)-PDGR promoter or other promoter-enhancer elements may be selected. Other enhancer sequences useful herein include the IRBP enhancer (Nicoud 2007, J Gene Med. 2007 December; 9(12):1015-23), immediate early cytomegalovirus enhancer, one derived from an immunoglobulin gene or SV40 enhancer, the cis-acting element identified in the mouse proximal promoter, etc.

In addition to a promoter, an expression cassette and/or a vector may contain other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A variety of suitable polyA are known. In one example, the polyA is rabbit beta globin, such as the 127 bp rabbit beta-globin polyadenylation signal (GenBank #V00882.1). In other embodiments, an SV40 polyA signal is selected. In certain embodiments, the poly A is a bovine growth hormone polyadenylation (bGH-polyA) signal.

Still other suitable polyA sequences may be selected. In certain embodiments, an intron is included. One suitable intron is a chicken beta-actin intron. In one embodiment, the intron is 875 bp (GenBank #X00182.1). In another embodiment, a chimeric intron available from Promega is used. However, other suitable introns may be selected. In one embodiment, spacers are included such that the vector genome is approximately the same size as the native AAV vector genome (e.g., between 4.1 and 5.2 kb). In one embodiment, spacers are included such that the vector genome is approximately 4.7 kb. See, Wu et al, Effect of Genome Size on AAV Vector Packaging, Mol Ther. 2010 January; 18(1): 80-86, which is incorporated herein by reference.

Selection of these and other common vector and regulatory elements are conventional and many such sequences are available. See, e.g., Sambrook et al, and references cited therein at, for example, pages 3.18-3.26 and 16.17-16.27 and Ausubel et al., Current Protocols in Molecular Biology, John Wiley & Sons, New York, 1989. Of course, not all vectors and expression control sequences will function equally well to express all of the transgenes as described herein. However, one of skill in the art can select among these, and other, expression control sequences without departing from the scope of this invention.

In certain embodiments, the expression cassette contains at least one miRNA target sequence that is a miR-183 target sequence. In certain embodiments, the vector genome or expression cassette contains an miR-183 target sequence that includes AGTGAATTCTACCAGTGCCATA (SEQ ID NO: 13), where the sequence complementary to the miR-183 seed sequence is underlined. In certain embodiments, the vector genome or expression cassette contains more than one copy (e.g., two or three copies) of a sequence that is 100% complementary to the miR-183 seed sequence. In certain embodiments, a miR-183 target sequence is about 7 nucleotides to about 28 nucleotides in length and includes at least one region that is at least 100% complementary to the miR-183 seed sequence. In certain embodiments, a miR-183 target sequence contains a sequence with partial complementarity to SEQ ID NO: 13 and, thus, when aligned to SEQ ID NO: 13, there are one or more mismatches. In certain embodiments, a miR-183 target sequence comprises a sequence having at least 1, 2, 3, 4, 5, 6, 7, 8, 9, or 10 mismatches when aligned to SEQ ID NO: 13, where the mismatches may be non-contiguous. In certain embodiments, a miR-183 target sequence includes a region of 100% complementarity which also comprises at least 30% of the length of the miR-183 target sequence. In certain embodiments, the region of 100% complementarity includes a sequence with 100% complementarity to the miR-183 seed sequence. In certain embodiments, the remainder of a miR-183 target sequence has at least about 80% to about 99% complementarity to miR-183. In certain embodiments, the expression cassette or vector genome includes a miR-183 target sequence that comprises a truncated SEQ ID NO: 13, i.e., a sequence that lacks at least 1, 2, 3, 4, 5, 6, 7, 8, 9, or 10 nucleotides at either or both the 5′ or 3′ ends of SEQ ID NO: 13. In certain embodiments, the expression cassette or vector genome comprises a transgene and one miR-183 target sequence. In yet other embodiments, the expression cassette or vector genome comprises at least two, three or four miR-183 target sequences.

In certain embodiments, the expression cassette contains at least one miRNA target sequence that is a miR-182 target sequence. In certain embodiments, the vector genome or expression cassette contains an miR-182 target sequence that includes AGTGTGAGTTCTACCATTGCCAAA (SEQ ID NO: 14). In certain embodiments, the vector genome or expression cassette contains more than one copy (e.g. two or three copies) of a sequence that is 100% complementary to the miR-182 seed sequence. In certain embodiments, a miR-182 target sequence is about 7 nucleotides to about 28 nucleotides in length and includes at least one region that is at least 100% complementary to the miR-182 seed sequence. In certain embodiments, a miR-182 target sequence contains a sequence with partial complementarity to SEQ ID NO: 14 and, thus, when aligned to SEQ ID NO: 14, there are one or more mismatches. In certain embodiments, a miR-183 target sequence comprises a sequence having at least 1, 2, 3, 4, 5, 6, 7, 8, 9, or 10 mismatches when aligned to SEQ ID NO: 14, where the mismatches may be non-contiguous. In certain embodiments, a miR-182 target sequence includes a region of 100% complementarity which also comprises at least 30% of the length of the miR-182 target sequence. In certain embodiments, the region of 100% complementarity includes a sequence with 100% complementarity to the miR-182 seed sequence. In certain embodiments, the remainder of a miR-182 target sequence has at least about 80% to about 99% complementarity to miR-182. In certain embodiments, the expression cassette or vector genome includes a miR-182 target sequence that comprises a truncated SEQ ID NO: 14, i.e., a sequence that lacks at least 1, 2, 3, 4, 5, 6, 7, 8, 9, or 10 nucleotides at either or both the 5′ or 3′ ends of SEQ ID NO: 14. In certain embodiments, the expression cassette or vector genome comprises a transgene and one miR-182 target sequence. In yet other embodiments, the expression cassette or vector genome comprises at least two, three or four miR-182 target sequences.

The term “tandem repeats” is used herein to refer to the presence of two or more consecutive miRNA target sequences. These miRNA target sequences may be continuous, i.e., located directly after one another such that the 3′ end of one is directly upstream of the 5′ end of the next with no intervening sequences, or vice versa. In another embodiment, two or more of the miRNA target sequences are separated by a short spacer sequence.

As used herein, as “spacer” is any selected nucleic acid sequence, e.g., of 1, 2, 3, 4, 5, 6, 7, 8, 9 or 10 nucleotides in length which is located between two or more consecutive miRNA target sequences. In certain embodiments, the spacer is 1 to 8 nucleotides in length, 2 to 7 nucleotides in length, 3 to 6 nucleotides in length, four nucleotides in length, 4 to 9 nucleotides, 3 to 7 nucleotides, or values which are longer. Suitably, a spacer is a non-coding sequence. In certain embodiments, the spacer may be of four (4) nucleotides. In certain embodiments, the spacer is GGAT. In certain embodiments, the spacer is six (6) nucleotides. In certain embodiments, the spacer is CACGTG or GCATGC.

In certain embodiments, the tandem repeats contain two, three, four or more of the same miRNA target sequence. In certain embodiments, the tandem repeats contain at least two different miRNA target sequences, at least three different miRNA target sequences, or at least four different miRNA target sequences, etc. In certain embodiments, the tandem repeats may contain two or three of the same miRNA target sequence and a fourth miRNA target sequence which is different.

In certain embodiments, there may be at least two different sets of tandem repeats in the expression cassette. For example, a 3′ UTR may contain a tandem repeat immediately downstream of the transgene, UTR sequences, and two or more tandem repeats closer to the 3′ end of the UTR. In another example, the 5′ UTR may contain one, two or more miRNA target sequences. In another example the 3′ may contain tandem repeats and the 5′ UTR may contain at least one miRNA target sequence.

In certain embodiments, the expression cassette contains two, three, four or more tandem repeats which start within about 0 to 20 nucleotides of the stop codon for the transgene. In other embodiments, the expression cassette contains the miRNA tandem repeats at least 100 to about 4000 nucleotides from the stop codon for the transgene.

See, PCT/US19/67872, filed Dec. 20, 2019, which is incorporated by reference herein and which claims priority to U.S. Provisional Patent Application No. 62/783,956, filed Dec. 21, 2018, which is hereby incorporated by reference. U.S. Provisional Patent Application No. 63/023,593, filed May 12, 2020, U.S. Provisional Patent Application No. 63/038,488, filed Jun. 12, 2020, and U.S. Provisional Patent Application No. 63/043,562, filed Jun. 24, 2020 are also incorporated by reference.

In another embodiment, a method of generating a recombinant adeno-associated virus is provided. A suitable recombinant adeno-associated virus (AAV) is generated by culturing a host cell which contains a nucleic acid sequence encoding an AAV capsid protein as described herein, or fragment thereof; a functional rep gene; a minigene composed of, at a minimum, AAV inverted terminal repeats (ITRs) and a heterologous nucleic acid sequence encoding a desirable transgene; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. Methods of generating a capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1, which are incorporated by reference herein.

Also provided herein are host cells transduced with an rAAV as described herein. Most suitably, such a stable host cell will contain the required component(s) under the control of an inducible promoter. However, the required component(s) may be under the control of a constitutive promoter. Examples of suitable inducible and constitutive promoters are provided herein, in the discussion below of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art. In another embodiment, the host cell comprises a nucleic acid molecule as described herein. In certain embodiments, the novel vectors described have improved productions (i.e. higher yields) compared to known capsids. For example, production of AAVrh91 vectors demonstrated improved yields compared to AAV1 and AAV6.

The minigene, rep sequences, cap sequences, and helper functions required for producing the rAAV described herein may be delivered to the packaging host cell in the form of any genetic element which transfers the sequences carried thereon. The selected genetic element may be delivered by any suitable method, including those described herein. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, 1993 J Virol., 70:520-532 and U.S. Pat. No. 5,478,745, among others. These publications are incorporated by reference herein.

### C. PHARMACEUTICAL COMPOSITIONS AND ADMINISTRATION

In one embodiment, the recombinant AAV containing the desired transgene and promoter for use in the target cells as detailed above is optionally assessed for contamination by conventional methods and then formulated into a pharmaceutical composition intended for administration to a subject in need thereof. Such formulation involves the use of a pharmaceutically and/or physiologically acceptable vehicle or carrier, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20. In another embodiment, the pharmaceutically acceptable carrier comprises a surfactant, such as perfluorooctane (Perfluoron liquid). The vector is formulated in a buffer/carrier suitable for infusion in human subjects. The buffer/carrier should include a component that prevents the rAAV from sticking to the infusion tubing but does not interfere with the rAAV binding activity in vivo.

In certain embodiments of the methods described herein, the pharmaceutical composition described above is administered to the subject intramuscularly (IM). In other embodiments, the pharmaceutical composition is administered by intravenously (IV). In other embodiments, the pharmaceutical composition is administered by intracerebroventricular (ICV) injection. In other embodiments, the pharmaceutical composition is administered by intra-cisterna magna (ICM) injection. In other embodiments, the pharmaceutical composition is administered by intraparenchymal injection. Other forms of administration that may be useful in the methods described herein include, but are not limited to, direct delivery to a desired organ (e.g., the eye), including subretinal or intravitreal delivery, oral, inhalation, intranasal, intratracheal, intravenous, intramuscular, subcutaneous, intradermal, and other parental routes of administration. Routes of administration may be combined, if desired.

As used herein, the terms “intrathecal delivery” or “intrathecal administration” refer to a route of administration via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cistema magna.

As used herein, the terms “intracisternal delivery” or “intracisternal administration” refer to a route of administration directly into the cerebrospinal fluid of the cistema magna cerebellomedularis, more specifically via a suboccipital puncture or by direct injection into the cistema magna or via permanently positioned tube.

The composition may be delivered in a volume of from about 0.1 μL to about 10 mL, including all numbers within the range, depending on the size of the area to be treated, the viral titer used, the route of administration, and the desired effect of the method. In one embodiment, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL. In another embodiment, the volume is about 1.5 mL. In another embodiment, the volume is about 2 mL. In another embodiment, the volume is about 2.5 mL. In another embodiment, the volume is about 3 mL. In another embodiment, the volume is about 3.5 mL. In another embodiment, the volume is about 4 mL. In another embodiment, the volume is about 5 mL. In another embodiment, the volume is about 5.5 mL. In another embodiment, the volume is about 6 mL. In another embodiment, the volume is about 6.5 mL. In another embodiment, the volume is about 7 mL. In another embodiment, the volume is about 8 mL. In another embodiment, the volume is about 8.5 mL. In another embodiment, the volume is about 9 mL. In another embodiment, the volume is about 9.5 mL. In another embodiment, the volume is about 10 mL.

An effective concentration of a recombinant adeno-associated virus carrying a nucleic acid sequence encoding the desired transgene under the control of the regulatory sequences desirably ranges from about 10⁷ and 10¹⁴ vector genomes per milliliter (vg/mL) (also called genome copies/mL (GC/mL)). In one embodiment, the rAAV vector genomes are measured by real-time PCR. In another embodiment, the rAAV vector genomes are measured by digital PCR. See, Lock et al, Absolute determination of single-stranded and self-complementary adeno-associated viral vector genome titers by droplet digital PCR, Hum Gene Ther Methods. 2014 April; 25(2):115-25. doi: 10.1089/hgtb.2013.131. Epub 2014 Feb. 14, which are incorporated herein by reference. In another embodiment, the rAAV infectious units are measured as described in S. K. McLaughlin et al, 1988 J. Virol., 62:1963, which is incorporated herein by reference.

Preferably, the concentration is from about 1.5×10⁹ vg/mL to about 1.5×10¹³ vg/mL, and more preferably from about 1.5×10⁹ vg/mL to about 1.5×10¹¹ vg/mL. In one embodiment, the effective concentration is about 1.4×10⁸ vg/mL. In one embodiment, the effective concentration is about 3.5×10¹⁰ vg/mL. In another embodiment, the effective concentration is about 5.6×10¹¹ vg/mL. In another embodiment, the effective concentration is about 5.3×10¹² vg/mL. In yet another embodiment, the effective concentration is about 1.5×10² vg/mL. In another embodiment, the effective concentration is about 1.5×10¹³ vg/mL. All ranges described herein are inclusive of the endpoints.

In one embodiment, the dosage is from about 1.5×10⁹ vg/kg of body weight to about 1.5×10¹³ vg/kg, and more preferably from about 1.5×10⁹ vg/kg to about 1.5×10¹¹ vg/kg. In one embodiment, the dosage is about 1.4×10⁸ vg/kg. In one embodiment, the dosage is about 3.5×10¹⁰ vg/kg. In another embodiment, the dosage is about 5.6×10¹¹ vg/kg. In another embodiment, the dosage is about 5.3×10¹² vg/kg. In yet another embodiment, the dosage is about 1.5×10¹² vg/kg. In another embodiment, the dosage is about 1.5×10¹³ vg/kg. In another embodiment, the dosage is about 3.0×10¹³ vg/kg. In another embodiment, the dosage is about 1.0×10¹⁴ vg/kg. All ranges described herein are inclusive of the endpoints.

In one embodiment, the effective dosage (total genome copies delivered) is from about 10⁷ to 10¹³ vector genomes. In one embodiment, the total dosage is about 10⁸ genome copies. In one embodiment, the total dosage is about 10⁹ genome copies. In one embodiment, the total dosage is about 10¹⁰ genome copies. In one embodiment, the total dosage is about 10¹¹ genome copies. In one embodiment, the total dosage is about 10¹² genome copies. In one embodiment, the total dosage is about 10¹³ genome copies. In one embodiment, the total dosage is about 10¹⁴ genome copies. In one embodiment, the total dosage is about 10¹⁵ genome copies.

It is desirable that the lowest effective concentration of virus be utilized in order to reduce the risk of undesirable effects, such as toxicity. Still other dosages and administration volumes in these ranges may be selected by the attending physician, taking into account the physical state of the subject, preferably human, being treated, the age of the subject, the particular disorder and the degree to which the disorder, if progressive, has developed. Intravenous delivery, for example may require doses on the order of 1.5×10¹³ vg/kg.

### D. METHODS

In another aspect, a method of transducing a target cell or tissue is provided. In one embodiment, the method includes administering a rAAV having an AAVrh91 capsid as described herein. As shown in the examples below, the inventors have shown that the AAV termed AAVrh91 effectively transduces heart (smooth muscle), CNS cells, and skeletal (striated) muscle. As described herein, vectors having an AAVrh91 capsid are capable of transducing a variety of cell and tissue types and exhibit unique tropisms that are dependent on the route of administration. In certain embodiments, the methods include systemic administration of a AAVrh91 vector. In certain embodiments, the AAVrh91 vector is delivered via a route of administration suitable to target a particular cell or tissue type. For example, AAVrh91 vectors have higher transgene expression levels than AAV6.2 in tissues such as the lung and pancreas following intrathecal administration. Likewise, enhanced expression in muscles tissue, relative to AAV6.2, has been observed for AAVrh91 following intrathecal administration.

In certain embodiments, provided herein is a method of transducing cells of the CNS (for example, one or more of neurons, endothelial cells, glial cells, and ependymal cells) comprising administering an rAAV having a AAVrh91 capsid. In one embodiment, intravenous administration is employed. In another embodiment, ICV administration is employed. In yet another embodiment, ICM administration is employed. In certain embodiments, provided herein is a method of delivering a transgene to a cell of the CNS, including but not limited to any of spinal cord, hippocampus, motor cortex, cerebellum, and motor neurons. The method includes contacting the cell with an rAAV having the AAVrh91 capsid, wherein said rAAV comprises a transgene. In another aspect, the use of an rAAV having the AAVrh91 capsid is provided for delivering a transgene to the CNS. In certain embodiments, the use of an rAAV having an AAVrh91 capsid is provided for delivering a transgene to ependymal cells or the choroid plexus. In certain embodiments, transduction of ependymal cells and/or choroid plexus results in enhanced levels of secretion of the transgene in the CNS.

In certain embodiments, AAVrh91 delivers a transgene to cells of the CNS at higher levels than observed with vectors having an AAV1 or AAV6.2 capsid. In certain embodiments, the higher levels of transduction are observed in one or more or ependymal cells, neurons and/or astrocytes. In certain embodiments, the use of an rAAV having an AAVrh91 capsid is provided for delivering a transgene to the brain parenchyma. Provided herein are uses of an AAVrh91 vector to target cells of the brain, such as astrocytes, at higher levels of transduction than achieved using an AAV9 vector. In certain embodiments, higher transduction levels are achieved in caudal sections of the brain, including frontal and temporal cortices. In certain embodiments, an AAVrh91 vector achieves higher levels of transduction, for example relative to AAV9, of neurons in the cortex, hippocampus, and/or striatum.

As discussed herein, the vectors comprising the AAV capsids described herein are capable of transducing heart tissue at high levels. Provided herein is a method of delivering a transgene to a heart cell. The method includes contacting the heart cell with an rAAV having the AAVrh91 capsid, wherein said rAAV comprises a transgene. In another aspect, the use of an rAAV having the AAVrh91 capsid is provided for delivering a transgene to heart. In certain embodiments, the method of delivering a transgene to cells of the heart comprises systemic delivery (e.g., IV administration) of a rAVV having an AAVrh91 capsid.

In certain embodiments, provided herein is a method of transducing skeletal muscle comprising administering an rAAV having the AAVrh91 capsid. AAVrh91 has similar, if not increased, transduction of skeletal muscle compared to AAV9. In certain embodiments, the method comprises delivering an AAVrh91 capsid to skeletal (striated) muscle. In certain embodiments, a method of delivering a transgene to skeletal muscle is provided. The method includes contacting skeletal muscle with an rAAV having the AAVrh91 capsid, wherein said rAAV comprises a transgene. In certain embodiments, the method of delivering a transgene skeletal muscle comprises systemic delivery (e.g., IV administration) of a rAAV having an AAVrh91 capsid.

In certain embodiments, the AAVrh91 vectors described as uses to reduce transduction of detarget expression in the liver of a subject. Thus, to avoid potential liver toxicity or reduce liver toxicity associated with AAV targeting of liver tissue, an AAVrh91 is used. In certain embodiments, the reduced liver toxicity is observed following systemic injection, in particular intravenous administration. In certain embodiments, the reduction in toxicity is relative to delivery of a vector with another capsid, such as a vector having an AAV9 capsid.

### Single Genome Amplification

AAV genomes have been traditionally isolated from within whole mammalian genomic DNA using PCR based methods: primers are used to detect conserved regions that flank the majority of the diverse VP1 (capsid) gene. The PCR products are then cloned into plasmid backbones and individual clones are sequenced using the Sanger method. Traditional PCR and molecular cloning based viral isolation methods are effective for recovering novel AAV genomes but the genomes recovered can be influenced by PCR-mediated recombination and polymerase errors. In addition, currently available next-generation sequencing technologies have allowed us to sequence viral genomes with unparalleled accuracy compared to the previously used Sanger technology. Provided herein is a novel, higher-throughput, PCR and next-generation sequencing based method of accurately isolating individual AAV genomes from within a viral population. This method, AAV-Single Genome Amplification (AAV-SGA), can be used to improve our knowledge of AAV diversity within mammalian hosts. Moreover, it has allowed us to identify novel capsids for use as vectors for gene therapy.

AAV-SGA has been validated and optimized to effectively recover individual AAV sequences from samples that contain a population of genomes. This technique has been previously used to isolate single HIV and HCV genomes from within human and nonhuman primate hosts. The genomic DNA samples that screen positive for AAV by capsid detection PCR are endpoint-diluted. The dilution at which PCR amplification yields less than 30% positive reactions, according to a Poisson distribution, with 80% confidence, contains a single amplifiable AAV genome. This procedure allows for the PCR amplification of viral genomes with a reduced chance of PCR-mediated recombination caused by template switching of the polymerase. The AAV-SGA PCR amplicons are sequenced using the Illumina MiSeq platform using 2×150 or 2×250 paired-end sequencing. This method allows for accurate de novo assembly of full length AAV VP1 sequences without concern of convergence of sequencing reads from a single sample containing multiple viruses that have regions of high homology.

The AAV-SGA technique has been successful for isolation of multiple novel AAV capsid sequences from rhesus macaque tissues. Multiple viruses from different clades of AAV have been identified from single samples; this demonstrates that a population of AAVs can exist in the host tissues. For example, capsids with sequence similarity to clades D, E, and the outlying “fringe” viruses were isolated from a single liver tissue sample.

The application to of SGA to AAV discovery has not been previously described. The approach addresses the template switching and polymerase error issues which can result in invalid AAV genome sequences. Additionally, the quality of the isolated genome is self-evident when the same sequence is recovered repeated from the same host sample as single isolates.

The following Examples are provided to illustrate various embodiments of the present invention. The Examples are not intended to limit the invention in any way.

### E. EXAMPLES

### Example 1: Materials and Methods

### Detection and Isolation of AAV Sequences

### Nonhuman Primate Tissue Sources

Rhesus macaques from the University of Pennsylvania colony were captive-bred and were of Chinese or Indian origin.

### Novel AAV Isolation

Genomic DNA was extracted (QIAmp DNA Mini Kit, QIAGEN) and analyzed for the presence of AAV DNA by using a PCR strategy to amplify a 3.1-kb full-length Cap fragment from NHP liver tissue specimens. A 5′ primer within a conserved region of the AAV Rep gene was used (AVINS, 5′-GCTGCGTCAACTGGACCAATGAGAAC-3′) (SEQ ID NO: 9) in combination with a 3′ primer located in a conserved region downstream of the AAV Cap gene (AV2CAS, 5′-CGCAGAGACCAAAGTTCAACTGAAACGA-3′) (SEQ ID NO: 10) for amplification of full-length AAV Cap amplicons. Q5 High-Fidelity Hot Start DNA Polymerase (New England Biolabs) was used to amplify AAV DNA using the following cycling conditions: 98° C. for 30 s; 98° C. for 10 s, 59° C. for 10 s, 72° C. for 93 s, 50 cycles; and a 72° C. extension for 120 s.

Template genomic DNA samples that resulted in a positive PCR reaction were subjected to AAV-Single Genome Amplification (AAV-SGA). Genomic DNA was endpoint diluted in 96-well plates such that fewer than 29 PCR reactions, using the same primers mentioned above, out of 96 yielded an amplification product. According to a Poisson distribution, the DNA dilution that yields PCR products in no more than 30% of wells contains one amplifiable AAV DNA template per positive PCR more than 80% of the time. AAV DNA amplicons from positive PCR reactions was sequenced using the Illumina MiSeq 2×150 or 2×250 paired end sequencing platforms and resulting reads were de novo assembled using the SPAdes assembler (cab.spbu.ru/software/spades). Sequence analysis was performed using NCBI BLASTn (blast.ncbi.nlm.nih.gov) and the Vector NTI AlignX software (Thermo Fisher).

### AAV Production and Titer Determination

AAV vectors used for in vitro analyses were produced by the triple transfection method in HEK293 cells. Vectors were produced at a 6-well plate scale using an adapted protocol from the previously described 1 cell stack scale HEK293 triple transfection method. The following modifications were made based on the reduced culture areas: 1) the plasmid ratio used was 2:1:0.1 (the helper plasmid: the trans plasmid: the cis plasmid, by mass); and 2) at harvest, no other treatment was performed beyond freeze/thaw (Lock, M., et al., Human gene therapy, 2010. 21: p. 1259-71). The vectors were packaged with the CB7.ffluciferase.rBG transgene. Cell lysates were collected and DNase I- and proteinase K-resistant vector genomes were titered by TaqMan qPCR amplification (Applied Biosystems, Foster City, Calif.), using primers and probes directed against the rabbit β-globin polyadenylation signal encoded in the transgene cassette.

### In Vitro Transduction Assays

After triple transfection and vector lysate harvest, 1×10¹⁰ GC/mL of each vector was serially diluted with fresh complete medium and then used to transduce Huh7 or HEK293 cells which were seeded at 1×10⁵ cells/well or 1.5×10⁶ cells/well, respectively, one day earlier. Luciferase activity was detected after D-Luciferin treatment (Promega, Madison, Wis.) with a luminometer (Biotek, Winooski, Vt.).

### In Vivo Characterization of Novel AAV Capsids in Rodents

### Animals

All animal protocols were approved by the Institutional Animal Care and Use Committee of the University of Pennsylvania. C56BL/6J mice were purchased from the Jackson Laboratory. For GFP reporter gene experiments, adult (6-8 weeks old) males were injected. Animals were housed in standard caging of two to five animals per cage. Cages, water bottles, and bedding substrates were autoclaved in the barrier facility, and cages were changed once per week. An automatically controlled 12-h light or dark cycle was maintained. Each dark period began at 7:00 p.m. (±30 min). Irradiated laboratory rodent food was provided ad libitum.

### Test Articles and Study Design

Mice received 1×10¹² GC per mouse of each vector in 0.1 mL intravenously (IV) via the lateral tail vein or were injected intracerebroventricularly (ICV) into the lateral ventricle of the brain at a dose of 1×10¹¹ GC in 5 uL per mouse. Three or five mice were dosed for each group.

Mice were euthanized by inhalation of CO₂ 14 days post injection. Tissues were collected, snap-frozen on dry ice for biodistribution analysis or were immersion-fixed in 10% neutral formalin, cryo-preserved in sucrose, frozen in OCT, and sectioned with a cryostat for GFP direct observation. Tissues used for endothelial cell transduction analysis were paraffin-embedded after necropsy.

### Reporter Gene Visualization

To observe direct GFP fluorescence, tissue samples were fixed in formalin for about 24 hours, briefly washed in PBS, equilibrated sequentially in 15% and 30% sucrose in PBS until they reached maximum density, and were then frozen in OCT embedding medium for the preparation of cryosections. Sections were mounted in Fluoromount G containing DAPI (Electron Microscopy Sciences, Hatfield, Pa.) as nuclear counterstain.

GFP immunohistochemistry was performed on paraffin-embedded tissue samples. Sections were deparaffinized with ethanol and xylene, boiled for 6 min in 10 mM citrate buffer (pH 6.0) for antigen retrieval, treated sequentially with 2% H₂O₂ for 15 min, avidin/biotin blocking reagents for 15 min each (Vector Laboratories), and blocking buffer (1% donkey serum in PBS+0.2% Triton) for 10 min. This was followed by incubation with primary antibodies for 1 hour and biotinylated secondary antibodies in blocking buffer for 45 min (Jackson Immunoresearch). The primary antibody, chicken anti-GFP (Abcam ab13970) and rabbit anti-CD31 (Abcam ab28364) endothelial cell marker, were used. A Vectastain Elite ABC kit (Vector Laboratories) was used following the manufacturer's instructions, with DAB as substrate, to visualize bound antibodies as brown precipitate.

For immunofluorescence, paraffin sections were deparaffinized and blocked after antigen retrieval with 1% donkey serum in PBS+0.2% Triton for 15 min followed by sequential incubation with primary (1 h) and fluorescence-labeled secondary antibodies (45 min, Jackson Immunoresearch) diluted in blocking buffer. Antibodies used were chicken anti-GFP (Abcam ab13970), rabbit anti-CD31 (Abcam ab28364), and mouse anti-NF-200 (clone RT97, Millipore CBL212). The primary antibodies were mixed together and the GFP and NF-200 antibodies were detected via FITC- and TRITC-labeled secondary antibodies, respectively. The signal for the rabbit antibody against CD31 was enhanced using a VectaFluor™ Excel Amplified DyLight® 488 Anti-Rabbit IgG kit according to the manufacturer's protocol (Vector Labs). LacZ gene expression detection based on X-gal staining was performed on skeletal muscle tissue sections using protocols demonstrated previously (Bell, P., et al., Histochemistry and Cell Biology, 2005. 124: p. 77-85). Fluorescence and brightfield microscopy images were taken with a Nikon Eclipse TiE microscope.

### Nonhuman Primate Transduction Evaluation of Barcoded Vector Transgenes

### Test Articles and Study Design

Five novel capsids and five control capsids (AAVrh.90, AAVrh91, AAVrh.92, AAVrh.93, AAVrh91.93, AAV8, AAV6.2, AAVrh32.33, AAV7, and AAV9) were used to package modified ATG-depleted self-complementary eGFP (dGFP) transgenes. Each unique capsid preparation contained the dGFP transgene with a corresponding unique 6 bp barcode prior to the polyadenylation sequence of the vector genome. The transgene contained a CB8 promoter and an SV40 polyadenylation sequence (AAVsc.CB8.dGFP.barcode.SV40). AAV vectors were produced and titrated by the Penn Vector Core as described before (see, e.g., Lock, M., et al. (2010) Hum. Gene Ther. 21:1259-71). HEK293 cells were triple transfected then the cell culture supernatant was harvested, concentrated, and purified with an iodixanol gradient. The purified vectors were titrated with droplet digital PCR using primers targeting the SV40 polyA sequence as described before (see, e.g., Lock, M., et al. (2014) Hum. Gene Ther. Methods 25:115-25).

The ten purified vectors were pooled at equal genome copy quantities for injection into two separate animals: total doses delivered were 2×10¹³ GC/kg via IV delivery and 3×10¹³ GC/animal via intra-cistema magna (ICM) delivery into the intrathecal space. Animals were sacrificed at 30 days post injection and all tissues were harvested in RNAlater (QIAGEN) for downstream transgene RNA expression analysis.

### Animals

All animal procedures were approved by the Institutional Animal Care and Use Committee of the University of Pennsylvania. Cynomolgus macaques (Macaca fascicularis) were donated from Bristol Meyers Squibb (USA). Animals were housed in an Association for Assessment and Accreditation of Laboratory Animal Care International-accredited Nonhuman Primate Research Program facility at the Children's Hospital of Philadelphia, Philadelphia, Pa. in stainless-steel squeeze back cages. Animals received varied enrichments such as food treats, visual and auditory stimuli, manipulatives, and social interactions.

A 10-year-old, male, 8 kg animal was used for the ICM study. A 6-year-old, male, 6.98 kg animal was used for the IV study. This animal was screened for the presence of AAV-neutralizing antibodies and was seronegative for AAV6, AAV8, and AAVrh32.33, at baseline. At baseline, this animal had neutralizing antibody titers of 1:5 and 1:10 against AAV7 and AAV9, respectively.

### ICM Injection Procedure

The anesthetized macaque was placed on an X-ray table in the lateral decubitus position with the head flexed forward. Aseptic technique was used to advance a 21 G-27 G, 1- to 1.5-inch Quincke spinal needle (Becton Dickinson, Franklin Lakes, N.J., USA) into the suboccipital space until the flow of CSF was observed. 1 mL of CSF was collected for baseline analysis. The correct placement of needle was verified by fluoroscopy (OEC 9800 C-arm; GE Healthcare, Little Chalfont, UK) in order to avoid potential injury of the brainstem. After CSF collection, a Luer access extension or a small-bore T port extension set catheter was connected to the spinal needle to facilitate dosing of 180 mg/mL Iohexol contrast media (GE Healthcare, Little Chalfont, UK). After verifying needle placement, a syringe containing the test article (volume equivalent to 1 mL plus the syringe volume and linker dead space) was connected to the flexible linker and injected over 30±5 s. The needle was removed, and direct pressure was applied to the puncture site.

### IV Injection Procedure

The macaque was administered with 10 mL of vector test article into a peripheral vein at a rate of 1 mL/min via an infusion pump (Harvard Apparatus, Holliston, Mass.).

### Transgene Expression Analysis

Whole tissue RNA was extracted from all RNALater-treated tissues using TRIzol according to the manufacturer's specifications (Life Technologies). Extracted RNA was treated with DNase I according to the manufacturer's protocol (Roche, Basel, Switzerland). RNA was purified using the RNeasy Mini Kit (QIAGEN). Reverse transcription synthesis of cDNA was performed using the Applied Biosystems High Capacity cDNA Reverse Transcriptase Kit (Life Technologies). Primers targeting regions flanking the 6 bp unique barcode were used to PCR amplify a 117 bp amplicon ((forward primer: GGCGAACAGCGGACACCGATATGAA (SEQ ID NO: 11), reverse primer: GGCTCTCGTCGCGTGAGAATGAGAA (SEQ ID NO: 12)) and Q5 High-Fidelity Hot Start DNA Polymerase (New England Biolabs) was used to perform the reactions using the following cycling conditions: 98° C. for 30 s; 98° C. for 10 s, 72° C. for 17 s, 25 cycles; and a 72° C. extension for 120 s. Amplicons were sequenced using the MiSeq Standard 2×150 bp sequencing platform (Illumina).

Barcode reads were analyzed using the fastq-join program from the Expression Analysis package (github.com/ExpressionAnalysis/ea-utils), cutadapt (cutadapt.readthedocs.io/en/stable/), the fastx toolkit package (hannonlab.cshl.edu/fastx_toolkit/), and R version 3.3.1. (cran.r-project.org/bin/windows/base/old/3.3.1/). Barcode expression count data from tissue samples were normalized to barcode counts from the sequenced injection vector material for each animal and barcode proportions from each tissue sample were plotted using GraphPad Prism version 7.04.

### ICM AAVrh91 Transduction Characterization Studies in NHP

### Animals and Study Design

All animal procedures were approved by the Institutional Animal Care and Use Committee of the University of Pennsylvania. Animals were housed in an Association for Assessment and Accreditation of Laboratory Animal Care International-accredited Nonhuman Primate Research Program facility at the Children's Hospital of Philadelphia, Philadelphia, Pa. in stainless-steel squeeze back cages. Animals received varied enrichments such as food treats, visual and auditory stimuli, manipulatives, and social interactions.

AAVrh91, AAV1, AAV8, and AAV9 capsids were packaged with plasmids expressing enhanced green fluorescent protein (eGFP) from the chicken beta actin (CB7) promoter (AAV.CB7.CI.eGFP.WPRE.rBG) using methods that were previously described (see, e.g., Lock, M., et al. (2010) Hum. Gene Ther. 21:1259-71 and Lock, M., et al. (2014) Hum. Gene Ther. Methods 25:115-25). A dose of 1.557×10¹³ GC was injected ICM into each animal. ICM injection methods are described above. Animals were sacrificed 28-31 days after injection and tissues were harvested on dry ice for DNA Vector Biodistribution studies. The brain was collected whole, trimmed, and sectioned using a brain mold according to the Recommended Practices for Sampling and Processing the Nervous System (Brain, Spinal Cord, Nerve, and Eye) during Nonclinical General Toxicity Studies. Pardo, et. al. (2012). STP Position Paper. Tissues were also collected, formalin-fixed, and were paraffin-embedded for histopathological analyses.

### Histological Analyses of Vector Transduction

For GFP immunohistochemistry (IHC), sections were deparaffinized with ethanol and xylene, boiled for 6 min in 10 mM citrate buffer (pH 6.0) for antigen retrieval, treated sequentially with 2% H₂O₂ for 15 min, avidin/biotin blocking reagents for 15 min each (Vector Laboratories), and blocking buffer (1% donkey serum in PBS+0.2% Triton) for 10 min. This was followed by incubation with a goat antibody against GFP (Novus Biologicals, NB100-1770, 1:500) overnight at 4° C. in blocking buffer and, after washing in PBS, biotinylated secondary anti-goat antibodies for 45 min (Jackson ImmunoResearch, 1:500) in blocking buffer. After washing in PBS a Vectastain Elite ABC kit (Vector Laboratories) was applied following the manufacturer's instructions, with DAB as substrate, to visualize bound antibodies as brown precipitate.

For immunofluorescence (IF), paraffin sections were pretreated similarly but without H₂O₂ and avidin/biotin blocking. The following primary antibodies were combined and sections incubated for 1 h at 37° C.: goat anti-GFP (Novus Biologicals, NB100-1770; 1:300-500), guinea pig anti-NeuN (Millipore, ABN90; 1:500), chicken anti-GFAP (Abcam, ab4674; 1:1000). This was followed after washing in PBS by incubation with fluorochrome-labeled secondary antibodies (FITC anti-goat, Cy5 anti-guinea pig, TRITC anti-GFAP; Jackson ImmunoResearch, 1 h at room temperature, 1:200). After washing in PBS, sections were mounted in Fluoromount G containing DAPI (Electron Microscopy Sciences) to counterstain nuclei.

### Vector Biodistribution Analysis

Tissue genomic DNA was extracted with QIAamp DNA Mini Kit (QIAGEN), and AAV vector genomes were quantified by real-time PCR using Taqman reagents (Applied Biosystems, Life Technologies) with primers/probe targeting the EGFP sequence of the vectors.

### Cellular Transduction Quantification Analyses in Central Nervous System Tissues (CNS)

IF slides were prepared as described above and scanned using an Aperio VERSA Scanning System. Whole slides were scanned at low magnification (1.25×) first to define the regions of interest. After the initial 1.25× scans, slides were scanned at 20× magnification with four different channels DAPI, FITC, TRITC and Cy5. Transduced neurons and astrocytes were quantified from the final 20× scans using co-staining detection algorithms developed with Visiopharm image analysis software v.2019.07.

Cryo-Electron Microscopy (cryoEM) for AAVrh91

CryoEM on AAVrh91 was performed at the University of Massachusetts Medical School Cryo-EM Core Facility. 3 μl of vector was added without dilution (3.37×10¹³ GC/ml) to a glow-charged R2/1 copper grid with a 2 nm thickness continuous carbon film (Quantifoil). After blotting for 7-8 seconds with filter paper at 22° C. and 95% relative humidity, the grid was frozen in liquid ethane slush using a Vitrobot Mark IV (Thermo Fisher Scientific). Two grids with slightly different ice thickness were obtained. 1584 movies were collected on grid 1, and 3675 movies were collected on grid 2 using a Talos Arctica electron microscope (Thermo Fisher Scientific) operating at 200 kV with a Gatan K3 direct detector (Gatan, Pleasanton, USA). The data were acquired using the SerialEM software. The pixel size was 0.435 Å/pix (bin=0.5), and the total dose was 36.984 electrons/Å², with 26 frames per movie. Images were collected with defocus in a range of −0.5 to −1.5 μm.

### AAVrh91 Structure Determination, Model Building, and Refinement

For grid 1 and grid 2, movies were motion-corrected using the Relion 3.0 implementation of MotionCor2 and binned to a final pixel size of 0.87 Å. After motion correction, we used ctffind4 to estimate the defocus of the micrographs and processed them using Relion 3.0. All processed images for grid 1 and 3664 processed images from grid 2 were then combined into a single dataset for a total of 5248 images. From this set we picked and sorted approximately 1,000 particles for two-dimensional (2D) classification. The best classes were used as templates for autopicking. A total of 283,818 particles from autopicking were sorted through one round of 2D classification to remove false positive and suboptimal particles, yielding 254,442 particles. The initial model was produced in C1 symmetry through ab initio model generation with Relion. We further sorted particles through three-dimensional (3D) classification with C1 symmetry and angular sampling into five classes. The three best classes were selected for a total of 173,558 particles. Using these particles, we performed 3D auto-refinement in C1 symmetry, applied icosahedral symmetry, and performed another round of 3D auto-refinement with icosahedral symmetry applied. We then performed CTF refinement and particle polishing on the refined particles. Final 3D auto-refinement and post-processing yielded the structure of AAVrh91 to 2.33 Å based on the Fourier shell correlation gold-standard cutoff of 0.143.

Initial models were generated from previously published structures of AAV1 (6JCR). These models were fit into the electron density and modified to reflect the AAVrh91 sequence in COOT. After the initial building step, we refined the model against the electron density maps using the phenix.real_space_refinement program included in the PHENIX software package. We generated full models with icosahedral non-crystallographic symmetry. We performed refinement under secondary structure and non-crystallographic symmetry (NCS) constraints using rigid-body fitting, global minimization, a local grid search, and anisotropic displacement parameter (ADP) refinement.

### Mass Spectrometry (MS) Analysis for Modification of Amino Acids on AAV Capsid Reagents

Ammonium bicarbonate, dithiothreitol (DTT), iodoacetamide (IAM) were purchased from Sigma (St. Louis, Mo.). Acetonitrile, formic acid, and trifluoroacetic acid (TFA), 8M guanidine hydrochloride (GndHCl), and trypsin were purchased Thermo Fisher Scientific (Rockford, Ill.).

### Trypsin Digestion

Stock solutions of 1 M DTT and 1.0 M iodoacetamide were prepared. Capsid proteins were denatured and reduced at 90° C. for 10 minutes in the presence of 10 mM DTT and 2M GndHCl. The samples were allowed to cool to room temperature then alkylated with 30 mM IAM at room temperature for 30 minutes in the dark. The alkylation reaction was quenched with the addition of 1 mL DTT. To the denatured protein solution add 20 mM Ammonium Bicarbonate, pH 7.5-8 at a volume that dilutes the final GndHCl concentration to 200 mM. Add trypsin solution for a 1:20 trypsin to protein ratio and incubate at 37° C. for 4 hours. After digestion, add TFA to a final of 0.5% to quench digestion reaction.

### LC-MS/MS

Online chromatography was performed with an Acclaim PepMap column (15 cm long, 300-μm inner diameter) and a Thermo UltiMate 3000 RSLC system (Thermo Fisher Scientific) coupled to a Q Exactive HF with a NanoFlex source (Thermo Fisher Scientific). During on-line analysis the column temperature was regulated to a temperature of 35° C. Peptides were separated with a gradient of mobile phase A (MilliQ water with 0.1% formic acid) and mobile phase B (acetonitrile with 0.1% formic acid). The gradient was run from 4% B to 6% B over 15 min, then to 10% B for 25 min (40 minutes total), then to 30% B for 46 min (86 minutes total). Samples are loaded directly to the column. The column size is 75 cm×15 um I.D. and is packed with 2 micron C18 media (Acclaim PepMap). Due to the loading, lead-in, and washing steps, the total time for an LC-MS/MS run was about 2 hours.

MS data were acquired using a data-dependent top-20 method for the Q Exactive HF, dynamically choosing the most abundant not-yet-sequenced precursor ions from the survey scans (200-2000 m/z). Sequencing was performed via higher energy collisional dissociation fragmentation with a target value of 1e5 ions determined with predictive automatic gain control and an isolation of precursors was performed with a window of 4 m/z. Survey scans were acquired at a resolution of 120,000 at m/z 200. Resolution for HCD spectra was set to 30,000 at m/z 200 with a maximum ion injection time of 50 ms and a normalized collision energy of 30. The S-lens RF level was set at 50, which gave optimal transmission of the m/z region occupied by the peptides from our digest. We excluded precursor ions with single, unassigned, or six and higher charge states from fragmentation selection.

### Data Processing

BioPharma Finder 1.0 software (Thermo Fisher Scientific) was used for analysis of all data acquired. For peptide mapping, searches were performed using a single-entry protein FASTA database with carbamidomethylation set as a fixed modification; and oxidation, deamidation, and phosphorylation set as variable modifications, a 10 ppm mass accuracy, a high protease specificity, and a confidence level of 0.8 for MS/MS spectra. The percent modification of a peptide was determined by dividing the mass area of the modified peptide by the sum of the area of the modified and native peptides. Considering the number of possible modification sites, isobaric species which are modified at different sites may co-migrate in a single peak. Consequently, fragment ions originating from peptides with multiple potential modification sites can be used to locate or differentiate multiple sites of modification. In these cases, the relative intensities within the observed isotope patterns can be used to specifically determine the relative abundance of the different modified peptide isomers. This method assumes that the fragmentation efficiency for all isomeric species is the same and independent on the site of modification. This approach allows the definition of the specific modified sites and also the potential combinations involved.

### Statistical Analyses

All statistical analyses were completed using Prism (GraphPad Software, San Diego, Calif., USA) version 7.04. Comparisons between two groups were performed using unpaired Student's t-tests and comparisons between multiple groups were performed using one-way analysis of variance (ANOVA, Kruskal-Wallis test and Dunn's multiple comparison's test).

### Histopathology

A board-certified veterinary pathologist who was blinded to the test article groups, established pathology severity scores defined as 0 for absence of lesion, 1 for minimal (<10%), 2 as mild (10-25%), 3 for moderate (25-50%), 4 for marked (50-95%), and 5 for severe (>95%). Scores were based on microscopic evaluation of hematoxylin and eosin (H&E)-stained tissues and represents the proportion of tissue affected by the lesion in an average high-power microscopy field.

### Vector Genome Copy and Transgene RNA Analysis

Tissue samples were snap frozen at the time of necropsy, and DNA was extracted using the QIAamp DNA Mini Kit (Qiagen, Valencia, Calif.). DNase treated total RNA was isolated from 100 mg of tissue. RNA was quantified by spectrophotometry and aliquots reverse transcribed to cDNA using random primers. Detection and quantification of vector GC in extracted DNA and relative nuclease HAO1 transcript expression in extracted RNA were performed by real-time PCR. Briefly, vector GC and RNA levels were quantified using primers/probe designed against the polyA sequence of the vector and a transgene-specific sequence, respectively.

### Quantification of GFP Protein Expression

Samples of diaphragm, heart, kidney, liver, lung, skeletal muscle (including biceps brachii, biceps femoris, deltoid, extensor carpi radialis, gastrocnemius, gluteus maximus, intercostal, pectoralis major, rectus abdominis, soleus, tibialis anterior, trapezius, and vastus lateralis), and spleen were homogenized and GFP protein levels determined by enzyme-linked immunosorbent assay (ELISA; abcam ab171581) according to the manufacturer's instructions. Briefly, tissues samples were homogenized in 500 μl of 1× Cell Extraction Buffer, centrifuged, and the supernatant was extracted. Diluted supernatants for each sample were added to the ELISA plate in duplicate and the assay was performed according to the manufacturer's instructions. Protein concentration of the supernatants was also determined by bicinchoninic acid (BCA) assay (Pierce™ BCA Protein Assay Kit, ThermoFisher). GFP protein levels were normalized to total protein levels per sample (μg GFP expression per pg protein).

### Immunohistochemistry

Tissue samples were fixed in 10% neutral buffered formalin, paraffin-embedded following standard protocols, and used for determination of eGFP expression by immunohistochemistry. Sections were deparaffinized through an ethanol and xylene series, boiled for 6 min in 10 mM citrate buffer (pH 6.0) to perform antigen retrieval, and blocked sequentially with 2% H₂O₂ (15 min), avidin/biotin blocking reagents (15 min each; Vector Laboratories), and blocking buffer (1% donkey serum in PBS+0.2% Triton X-100 for 10 min), followed by incubation with primary antibody against GFP (goat antibody NB100-1770, Novus Biologicals; diluted 1:500) overnight at 4° C. Sections were incubated with a biotinylated anti-rabbit secondary antibody (diluted 1:500, 45 min; Jackson ImmunoResearch) diluted in blocking buffer. A Vectastain Elite ABC kit (Vector Laboratories) using 3,3′-Diaminobenzidine (DAB) as substrate enabled visualization of bound antibodies as brown precipitate.

### Quantification of GFP Expression by IHC Image Analysis

GFP expression was quantitated from anti-GFP antibody immunolabeled sections from the heart, liver, and gastrocnemius skeletal muscle. Up to three immunolabeled sections per animal were scanned on an Aperio AT2 scanner (Leica Biosystems), and between five and ten regions of interest were selected for quantitation of GFP signal using ImageJ software (version 1.53c). GFP signal background was established using naïve controls; GFP signal exceeding background was quantitated and then normalized to section area.

### Seroprevalence of AAVrh91 in the Human Population

100 random human serum samples were acquired from Lee Biosolutions (Maryland Heights, Mo.). NAb titers to AAV2, AAV8, AAV9, AAVrh32.33, and AAVrh91 were determined, as described previously (Calcedo et al., 2009).

### Example 2: AAV-Single Genome Amplification (AAV-SGA)

Adeno-associated viruses (AAVs) are single-stranded DNA parvoviruses that are non-pathogenic and weakly immunogenic which make them effective candidates as vectors for gene therapy. Since the discovery of the first generation of AAVs (AAV1-6), our lab has led the effort to isolate a large number of viruses from a variety of higher primate species. This second generation of AAVs identified here were isolated using bulk PCR-based techniques using primers against conserved regions that were specific for primate-derived AAV genomes. Using AAV-SGA we have explored the genetic variation of AAVs in their natural mammalian hosts (FIG. 1).

AAV-SGA is a powerful technique that can be used to isolate single viral genomes from within a mixed population with high accuracy. In this study, we used AAV-SGA to identify novel AAV genomes from rhesus macaque tissue specimens. The novel viral isolates were genetically diverse and can be classified into clades D, E, and the Fringe clade (FIG. 2).

### Analysis of Vector Yield and In Vitro Transduction

All novel capsid sequences were used to produce gene delivery vectors. Each capsid VP1 sequence was cloned into a trans plasmid containing the standard AAV2 Rep gene. This trans plasmid was used in combination with various cis plasmids containing the vector transgene as well as the adenovirus helper plasmid for the HEK293 cell triple transfection vector production method. Purified vector titers were measured by droplet digital PCR after DNAse I treatment to determine the quantity of vector-encapsidated transgenes.

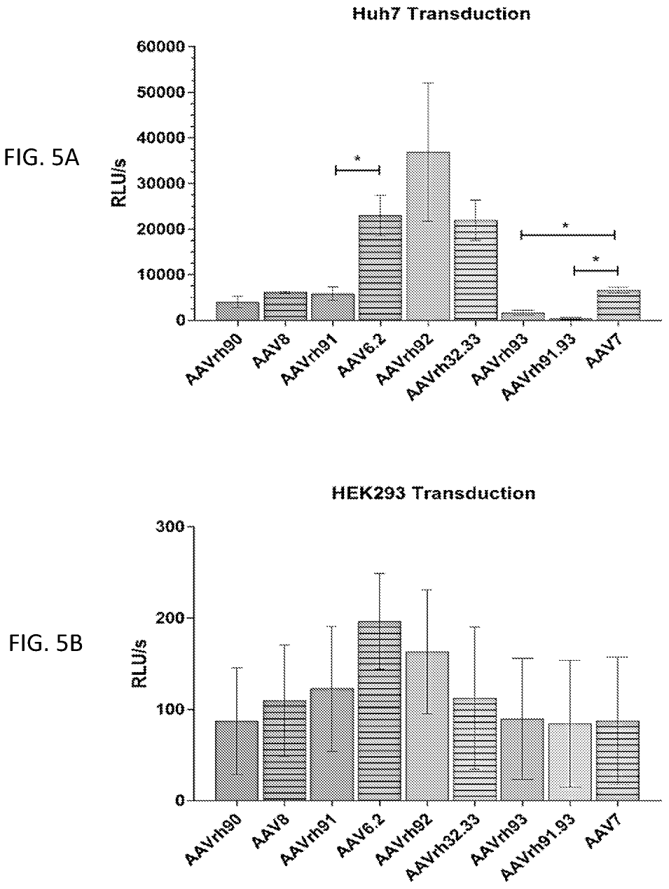

Using vectors containing the firefly luciferase transgene under the control of a ubiquitous promoter (CB7), we tested the novel capsids' in vitro transduction abilities in two human cell types: Huh7, a liver-derived cell line, and HEK293, a kidney-derived cell line. The vectors largely transduced the Huh7 cells with higher efficiencies than the HEK293 cells. In Huh7 cells, AAV6.2 and AAV7 both displayed significantly higher luciferase activity, a direct readout of transduction levels, than their novel capsid counterparts (FIG. 5A). All capsids transduced HEK293 cells at similarly low levels at the doses used (FIG. 5B).

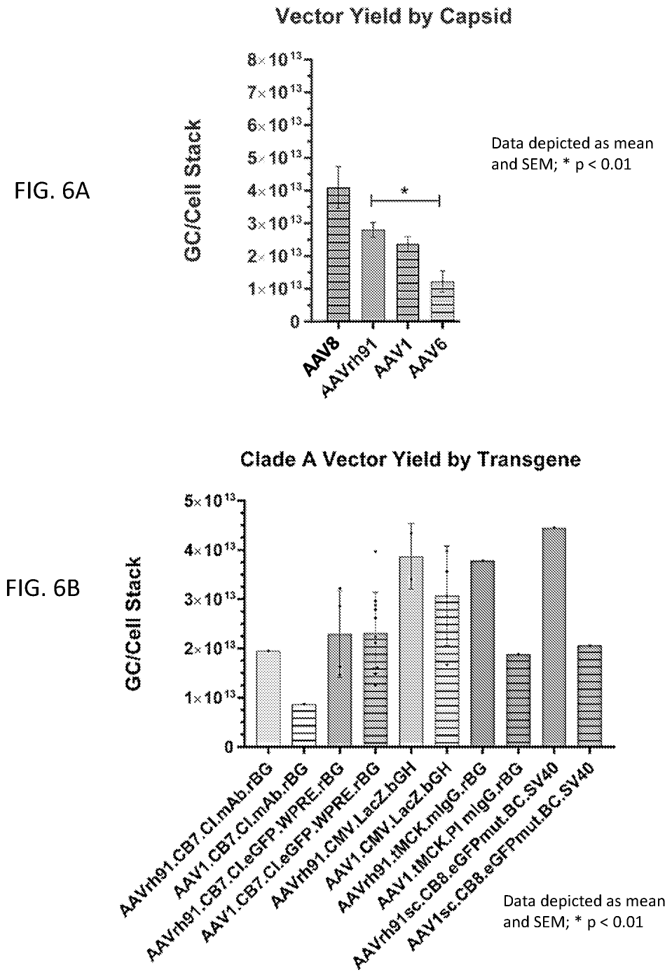

Novel capsids packaged transgene at similar efficiencies to their clade controls with the exception of AAVrh91 (FIG. 6A). AAVrh91-based vectors produced vector at significantly higher yields than AAV6-based vectors. When considering the effect of the type of packaged transgene on vector production in AAVrh91 and AAV1 capsids, we observed equal or one- to two-fold higher titers of AAVrh91 preparations than AAV1 preps containing the same transgenes, though tests for statistical significance were unable to be performed in all groups due to low replicate numbers (FIG. 6B).

AAVrh91 capsids were analyzed for deamidation and other modifications as previously described (see PCT/US19/019804 and PCT/US19/2019/019861). As shown in FIG. 7A, FIG. 7B, and FIG. 7C, the results indicated that AAVrh91 has three amino acids that are highly deamidated (N57, N383, and N512), which correspond to asparagines in asparagine-glycine pairs (numbering of AAVrh91 as in SEQ ID NO: 2). Lower deamidation percentages were consistently observed in residues N303, N497, and N691, as well as phosphorylation at S149.

### In Vivo Transduction of Novel AAV Capsids in Rodents

Next, we characterized the tissue tropism of the five new capsids in mice. All capsids were produced as gene delivery vectors containing ubiquitous promoters, CB7 or CMV, and either an enhanced green fluorescent protein (eGFP) or a β-galactosidase (LacZ) reporter transgene for testing in three mouse experiments.

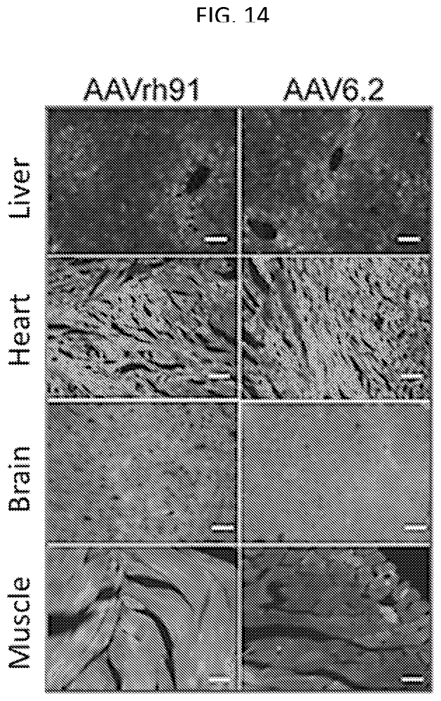

To test the vectors for their systemic transduction capabilities, we injected adult C57BL/6 mice via the intravenous (IV) tail vein route of administration (ROA). The vectors contained a CB7.eGFP transgene and were injected at a dose of 10¹² genome copies (GC) per mouse. Immunofluorescence microscopy of liver, heart, brain, and skeletal muscle showed similar trends in eGFP expression for AAVrh91 and AAV6.2 vectors (FIG. 14).

In order to bypass the BBB and promote transduction in the CNS tissues, we injected each CB7.eGFP vector with the ICV ROA into the CSF-containing lateral ventricle of adult C57BL/6 mice. All capsids, except for the clade A vectors, were administered at a dose of 1×10¹¹ GC per mouse. Clade A vectors were dosed at 6.9×10¹⁰ GC per mouse. Due to the low manufacturing yields of AAV6.2, we were unable to achieve an adequate vector concentration for this group.

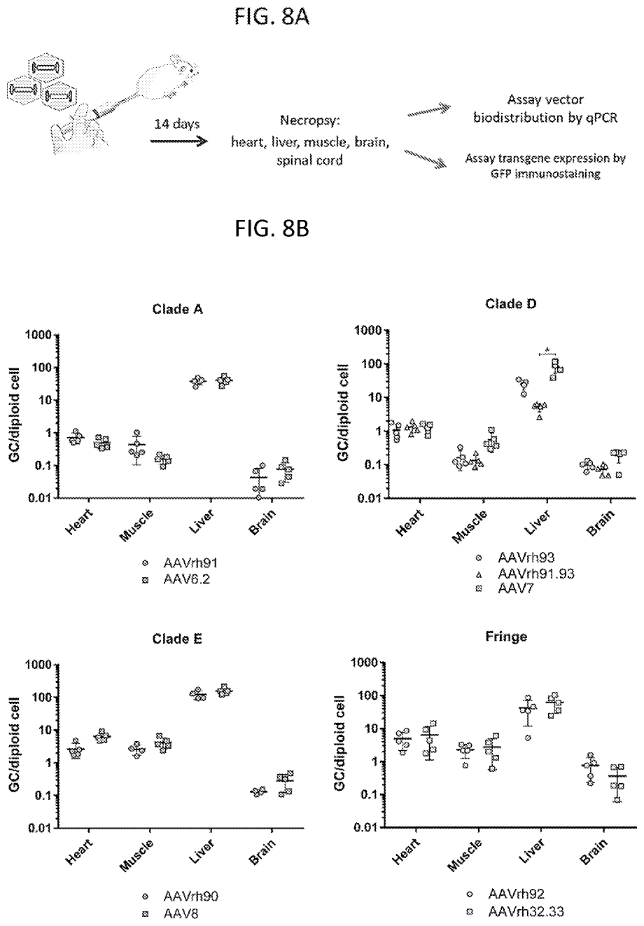

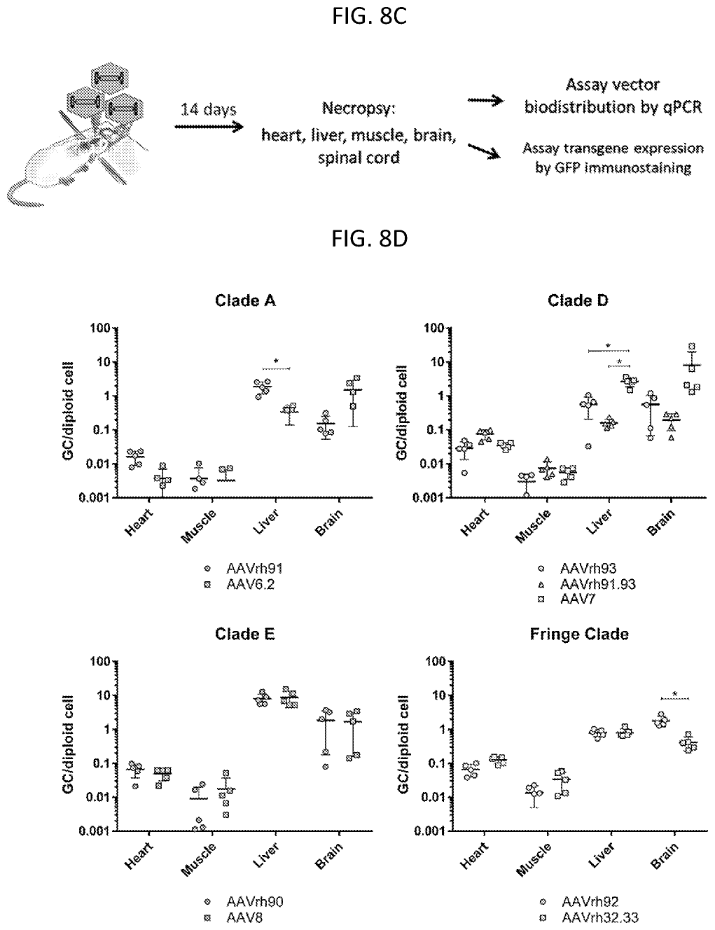

Fourteen days after injection, we assayed the biodistribution of the vector genomes in liver, heart, skeletal muscle, and most importantly, brain (FIG. 8D). On average, brain GC levels of AAV6.2 and AAV7 were higher than their novel capsid counterparts, AAVrh91, and AAVrh93 and AAVrh91.93, respectively; however these data were not statistically significant. We also observed that more GCs of AAVrh91 escaped into the periphery after delivery than the control capsid, AAV6.2, as indicated by the higher quantities of AAVrh91 vector genomes found in the liver (FIG. 8D).

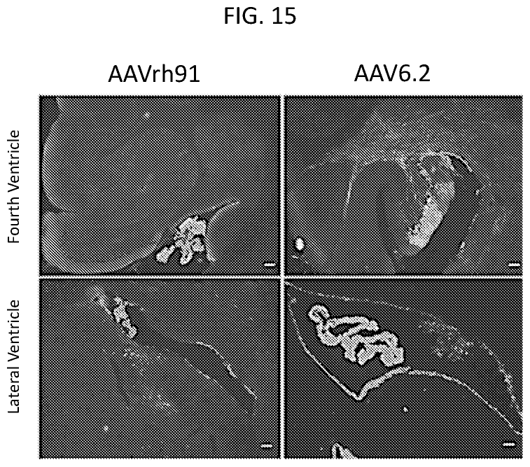

We qualitatively analyzed transgene expression in the ICV injected brains by direct fluorescence and observed variable transduction levels between the novel capsids and controls. The clade A vectors, AAVrh91 and AAV6.2 showed marked transduction of the choroid plexus and ependymal cells of the ventricles (FIG. 15).

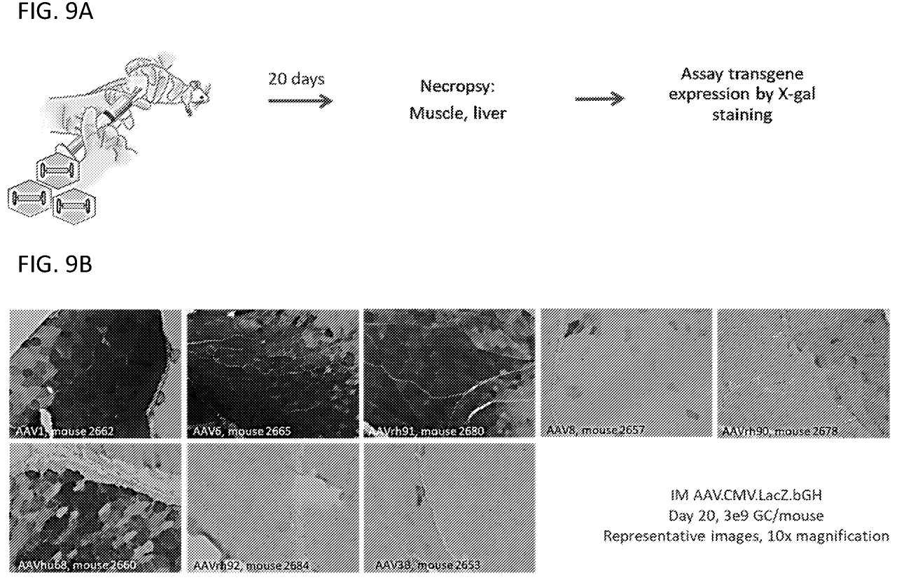

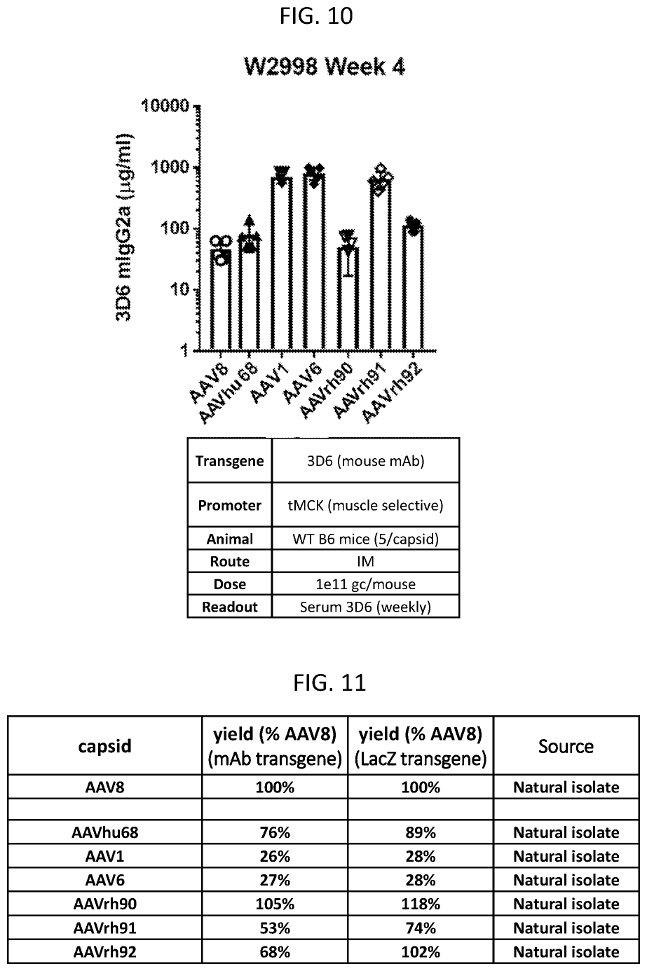

Finally, we tested vector delivery by the intramuscular ROA for the transduction of skeletal myocytes. For this study, we injected CMV.LacZ transgene containing vectors at a dose of 3×10⁹ GC per adult C57BL/6 mouse. Microscopy of tissues after β-galactosidase detection revealed uniformly strong myocyte transduction by clade A vectors, AAVrh91, AAV1, and AAV6. In contrast, at this dose, AAV8 showed poor transduction of muscle tissues (FIG. 9B). IM delivery via AAVrh91 also resulted in high levels of detectable mAb in serum (FIG. 10). FIG. 11 shows yields for various preparations of mAb and LacZ vectors. For both transgenes, AAVrh91 had higher yields compared to AAV1 and AAV6.

Overall, these studies showed that the novel AAVrh91 capsid is capable of transducing a variety of cell and tissue types in mice and exhibits unique tropisms that are dependent on the ROA.

### Example 3: Transduction Evaluation of Novel AAV Natural Isolates in Nonhuman Primates Using a Barcoded Transgene System

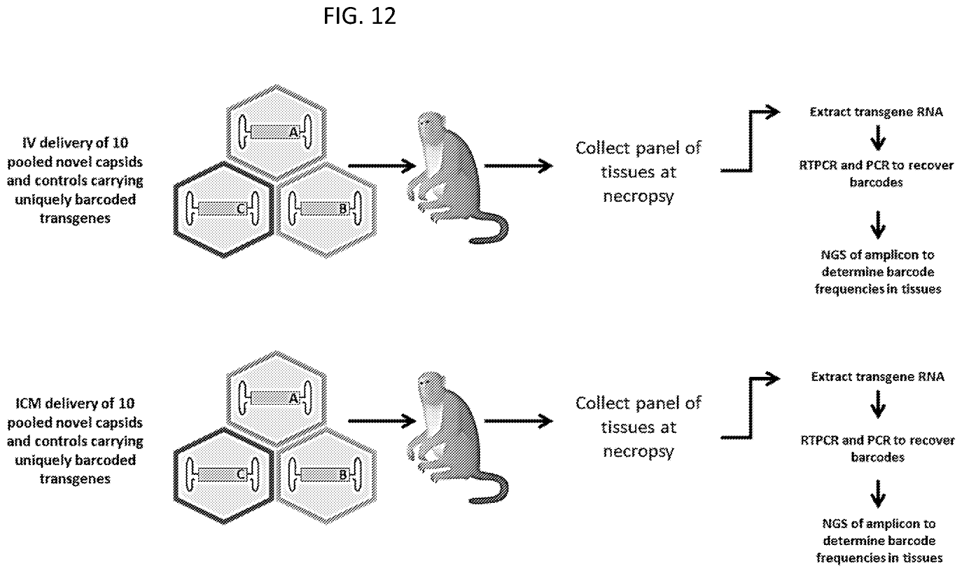

AAV vectors have been shown to be safe and effective gene transfer vehicles in clinical applications, yet they can be hindered by preexisting immunity to the virus and can have restricted tissue tropism. We demonstrated that a barcoded transgene method is effective to compare transduction of various tissues in a single animal by multiple AAV serotypes simultaneously. This technique reduces number of animals used and prevents foreign transgene-related immune responses. Accordingly, the novel capsids and their respective prototypical clade member controls (AAV6.2, AAV7, AAV8, AAVrh32.33, and AAV9) were made into vectors containing a modified eGFP transgene and unique six base pair barcodes prior to the polyA signal of the transcript (FIG. 12). The transgene was modified by deletion of ATG sequence motifs to prevent polypeptide translation and consequent immune response towards a foreign protein. Vectors were pooled at equal quantities and injected IV or ICM in cynomolgus macaques (total doses: 2×10¹³ GC/kg IV and 3×10¹³ GC ICM) to assess systemic and central nervous system transduction patterns of the novel capsids. All expression data were normalized to the actual input ratios to control for this slight variation in pooled proportions.

We administered the pooled vectors to two cynomolgus macaques using two different ROAs. To assay systemic transduction of the novel AAV capsids, we intravenously injected the first animal with a total dose of 2×10¹³ GC/kg of the pooled vector mixture. We additionally utilized an intrathecal (IT) delivery approach via intracistema magna (ICM) injection to deliver a vector dose of 3×10¹³ GC into the CSF of the second NHP for the direct targeting of CNS tissues. Thirty days after the vectors were delivered, transgene expression was analyzed from various tissues in each animal by extracting transgene RNA and subsequently quantifying barcode frequencies corresponding to each vector from each sample relative to the injection material.

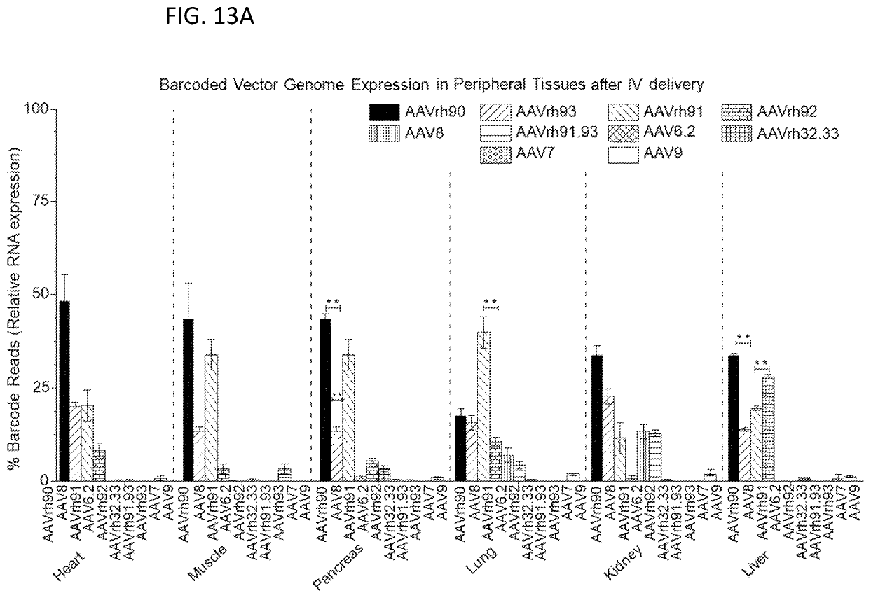

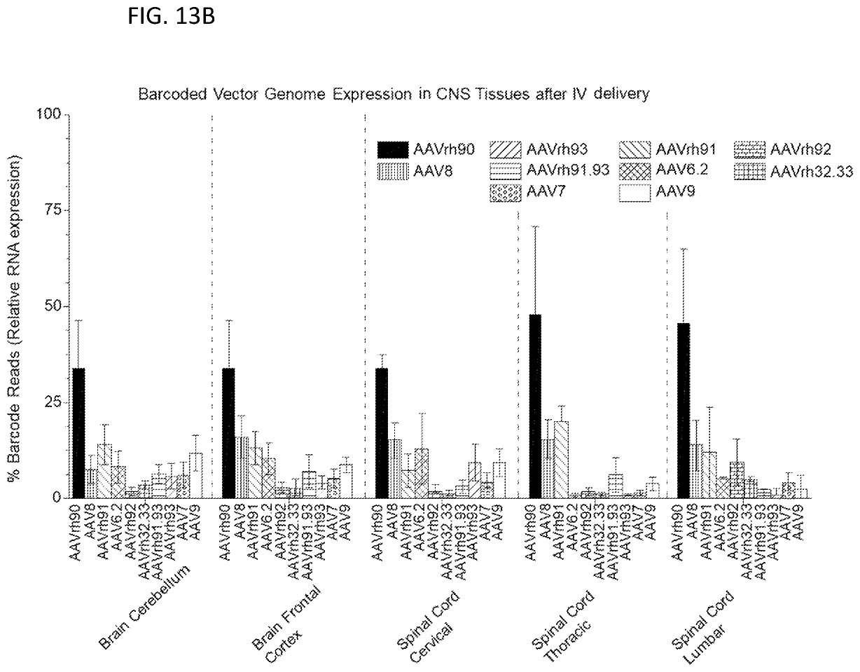

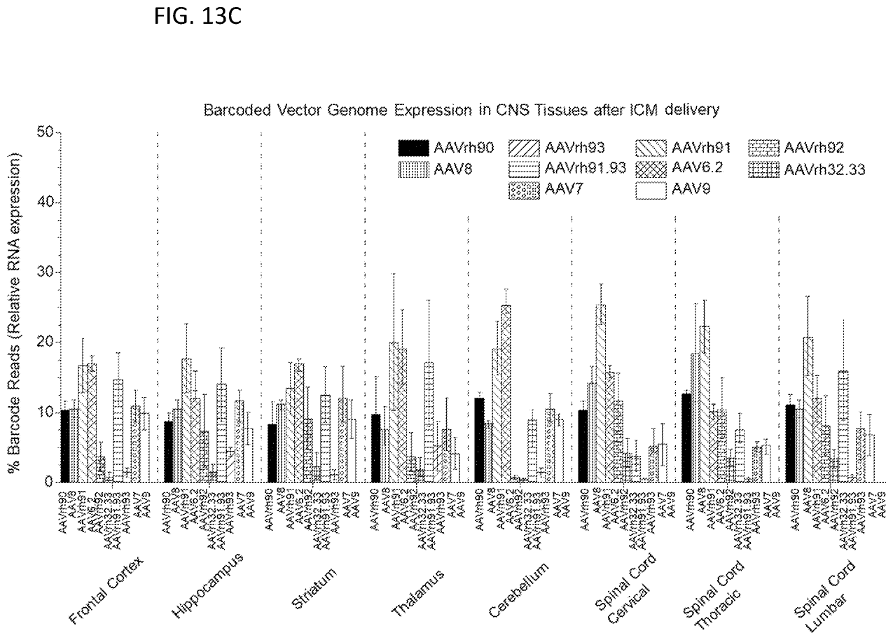

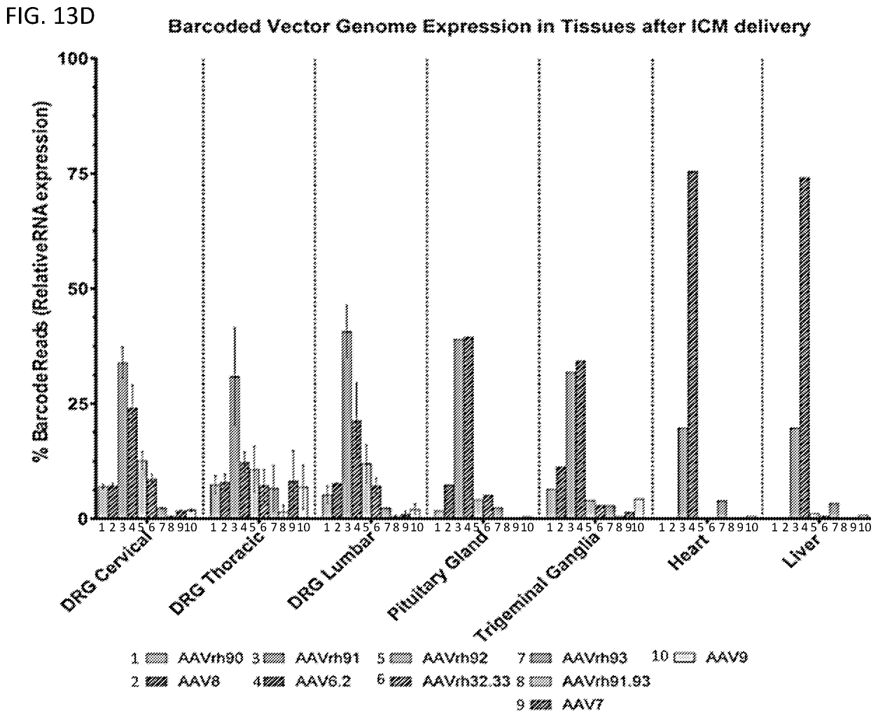

Interestingly, in the lung and pancreas tissues, AAVrh91 had higher transgene expression levels than AAV6.2 (FIG. 13A). We also observed that AAVrh91 transduced muscle tissue at higher levels than AAV6.2, but this was not as significant as its transduction enhancement in the pancreas or lung. Due to this animal having low levels of preexisting neutralizing antibodies to AAV7 and AAV9 at the time of injection (titers of 1:5 and 1:10, respectively), the barcode frequencies for the clade D and F capsids in all tissues were extremely low. On average, only 0.3-7% of all barcodes originated from AAV7, AAV9, AAVrh93, and AAVrh91.93 transgenes.

In the animal that was administered vector by the ICM ROA, clade A vectors AAVrh91 and AAV6.2 demonstrated high relative transduction frequencies in both tissues of the CNS as well as in tissues of the periphery, indicating that a proportion of the vectors entered systemic circulation after ICM delivery (FIG. 13C and FIG. 13D). This animal also had low levels of preexisting serum neutralizing antibodies against AAV7, AAV8, and AAV9 at titers of 1:10, 1:5, and 1:5, respectively.

These studies allowed us to efficiently assess the relative tissue tropism of the novel AAV capsids in individual NHPs and highlighted AAVrh91 as a potential vector for systemic and CNS targeting gene therapy applications.

### Example 4: AAVrh91 Displays a Strong CNS Transduction Profile after Intrathecal Delivery

Using the molecular barcoded transgene method to assay overall AAV vector tissue transduction is an effective way to screen relative expression levels in various organs. However, it can be technically complex to assess cellular tropism within tissues as there can be many different vectors transducing the same cells. Additionally, when pooling multiple vectors, the dose of individual vectors can be reduced to subclinical levels, which makes it difficult to assess the capsid's utility for translational applications.

In order to fully evaluate the cellular tropism within the CNS of the AAVrh91 vector, we generated vectors containing the CB7.eGFP transgene by the triple transfection method in HEK293 cells and injected rhesus macaques via ICM injection with 1.6×10¹³ GC of vector. AAV1 and AAV9 vectors containing the same transgene were also administered to two additional groups as controls as both vectors are well-studied; AAV1 is in the same clade as AAVrh91 and AAV9 is the current gold standard CNS tropic vector. Thus, we sought to compare the transduction efficiencies of the three capsids in a translationally relevant model organism.

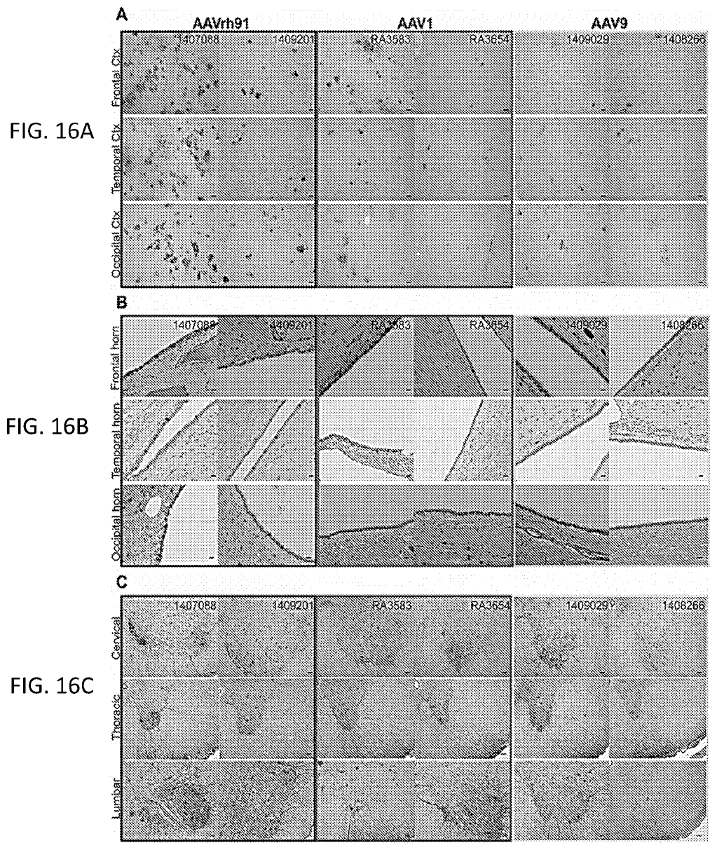

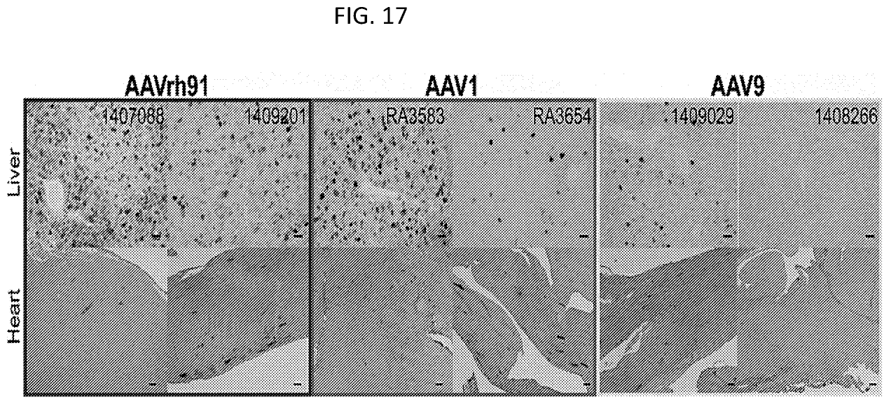

Approximately four weeks following ICM injection, we assessed transgene expression by GFP immunohistochemistry. We observed widespread levels of AAVrh91 vector-mediated gene expression in the frontal, temporal, and occipital cortices of the brain at levels higher than in AAV9 (FIG. 16A). The CSF-producing ependymal cells of the lateral ventricle were also strongly transduced by both clade A vectors, AAVrh91 and AAV1. In contrast, we were unable to see significant transduction of this cell type in animals administered AAV9 vector (FIG. 16B). GFP expression in the motor neurons of the spinal cord were similarly transduced by all three vectors with stronger GFP staining present in the lumbar segments (FIG. 16C). Interestingly, notable staining of GFP expression was observed in the liver and heart tissues of the animals administered AAVrh91 and AAV1, indicating that a proportion of vector entered systemic circulation from the CSF. Transduction of these peripheral tissues was weaker in AAV9 animals (FIG. 17).

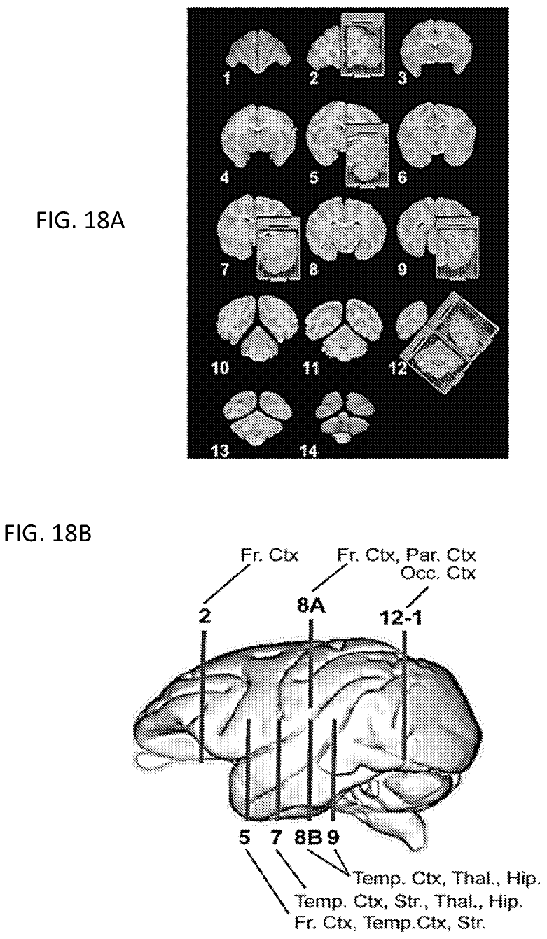

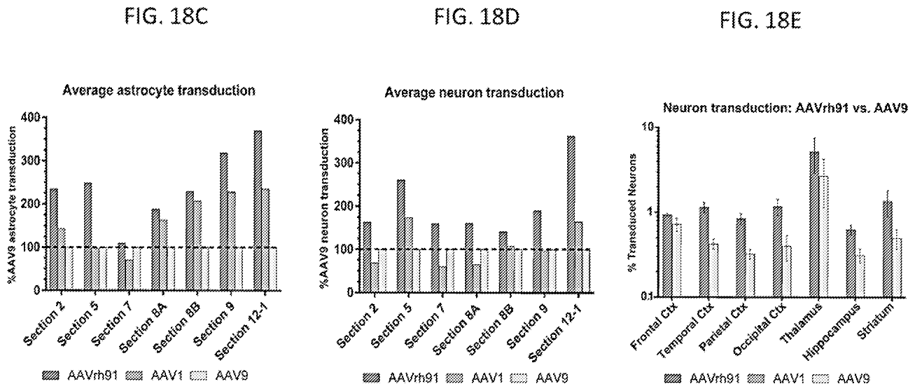

Next, we evaluated the cellular tropism of AAVrh91 in comparison to AAV1 and AAV9 using immunofluorescence cell quantification analyses. The mammalian brain is composed of two major cell types: neurons and glia. Using the glial fibrillary acidic protein (GFAP) and the neuronal nuclear protein (NeuN) markers we were able to stain for astrocytes (the major type of glial cell) and neurons, respectively, in brain tissue sections (FIG. 18A and FIG. 18B). We quantified cells that were stained with the DAPI nuclear stain and transduced GFP along with either GFAP or NeuN to determine the number of transduced astrocytes and neurons that were present in the brain.

We found that, on average, AAVrh91 transduced astrocytes at rates approximately 2-4-fold higher than AAV9 in most regions of the brain with a marked increase in transduction from the rostral to caudal regions. AAV1 transduced astrocytes at about 2 fold higher levels than AAV9 in caudal sections 8B, 9, and 12-1 but levels were more similar to AAV9 in sections 2, 5, and 7 which contain primarily frontal and temporal cortices (FIG. 18C). In contrast, the difference between AAVrh91 and AAV9 neuronal transduction was less with the former transducing at 1.5-2.5-fold higher levels than the latter (FIG. 18D). When stratified by specific brain region, we observed a similar trend overall, with about 1% of neurons in the cortex, hippocampus, and striatum transduced by AAVrh91 and 0.25-0.7% transduction by AAV9. Interestingly, the thalamus had much higher levels of transduction by both vectors than in the rest of the brain regions that were assessed (FIG. 18D).

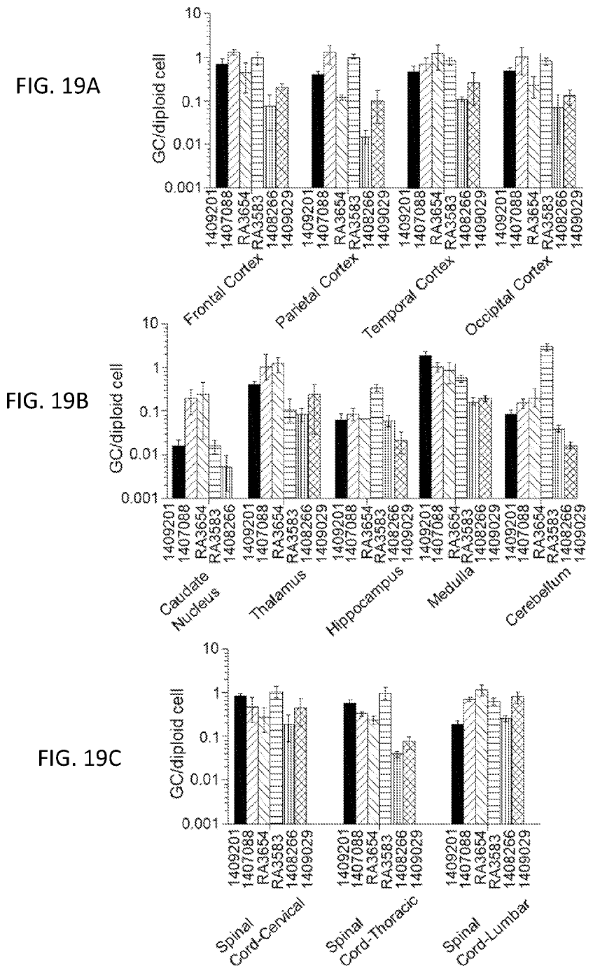

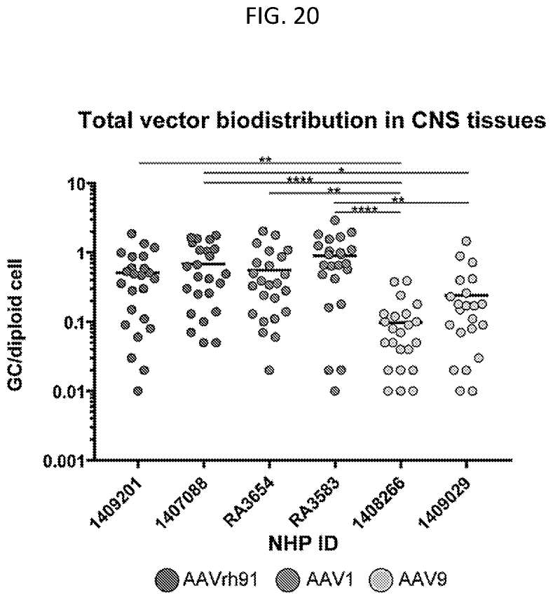

Biodistribution of vector genomes in all groups was assayed by qPCR. We found that AAVrh91 had the highest GC levels in most tissues that were screened from the CNS. AAV9 transduced tissues had a reduction of GC quantity of approximately one log in most tissues with the exception of the spinal cord which showed comparable GC presence in all groups (FIG. 19A-FIG. 19C). When considering average biodistribution of GCs in all of the CNS tissues that were screened, the animals that received AAVrh91 and AAV1 had significantly higher transduction levels than the animals that received AAV9 vector (FIG. 20).

Interestingly, the four animals that received the clade A GFP-expressing vectors displayed DRG and peripheral nerve pathology at necropsy which is indicative of AAV-mediated DRG toxicity. We found that the animals that had the highest transduction levels, AAVrh91 and AAV1, had overall higher grades of pathology in various peripheral nerves, DRGs, spinal cord regions, and the liver. Notably, one AAV1 dosed NHP, RA3654, exhibited mild clinical findings at study day 21: conscious proprioceptive deficits in both hind legs and hind limb ataxia. These clinical findings were resolved for the remainder of the study (days 22-30) after administering corticosteroids (prednisolone).

The studies described above provide a comprehensive analysis of novel AAV capsids that were isolated from natural sources and tested as in vitro and in vivo gene transfer vectors. Our novel capsids had amino acid sequence variation from control capsids in both the surface-exposed HVRs as well as in the structurally internal VP1 and VP2 unique regions. This diversity in sequence could allow for differential binding to host cell receptors which leads to variation in tissue tropism between different capsids. Additionally, the differences in sequence within the VP1 and VP2 unique regions could be contributing to discrepancies in trafficking of the vectors as these regions are attributed to interacting with various cytoplasmic components that mediate transgene delivery to the nucleus. Further studies using capsid mutagenesis techniques could elucidate the effect of the amino acid variations on AAV tropism and trafficking.

The differences between novel capsids and controls could also lead to differences in vector packaging. Interestingly, despite being only 1.1% different in VP1 protein sequence, we discovered that, based on vector yield, AAVrh91 vectors packaged transgene at significantly higher levels than AAV6.2 based vectors. We also found that AAVrh91 packages transgene at levels higher than AAV1.

AAV9 is one of the most well studied AAV capsids for its utility as a CNS tropic vector and it is considered the gold-standard for CNS gene therapy. In mice, it has been shown to be able to cross the BBB and transduce cells of the brain and spinal cord at high efficiencies after intravenous delivery. Also, there have been numerous studies demonstrating its effectiveness at localized CNS transduction after IT delivery into the CSF in both small and large animal models, though its transduction in the brain is diffuse. Here, we have identified a novel AAV capsid that effectively targets the primate CNS, AAVrh91. Its unique ependymal cell transduction phenotype could be of great use in treating disorders where a secreted transgene is required as this cell type can release transgene into the CSF that will then be circulated throughout the entire ventricular system. Though we also observed this ependymal cell transduction pattern using AAV1, AAVrh91 had higher overall brain transduction levels and has a better manufacturing profile. Interestingly, we observed a higher frequency of transduced cells in the liver and heart tissues in the AAVrh91 and AAV1 groups in comparison to the AAV9 group. AAVrh91 also exhibits efficient parenchymal transduction that is at least comparable to AAV9. Overall, with GC biodistribution and transduction levels greater than that of AAV9 in the majority of brain regions tested, AAVrh91 should be strongly considered for IT delivery of therapeutic transgenes for translational gene therapy studies in place of AAV9.

### Example 5: Seroprevalence of AAVrh91 in the Human Population

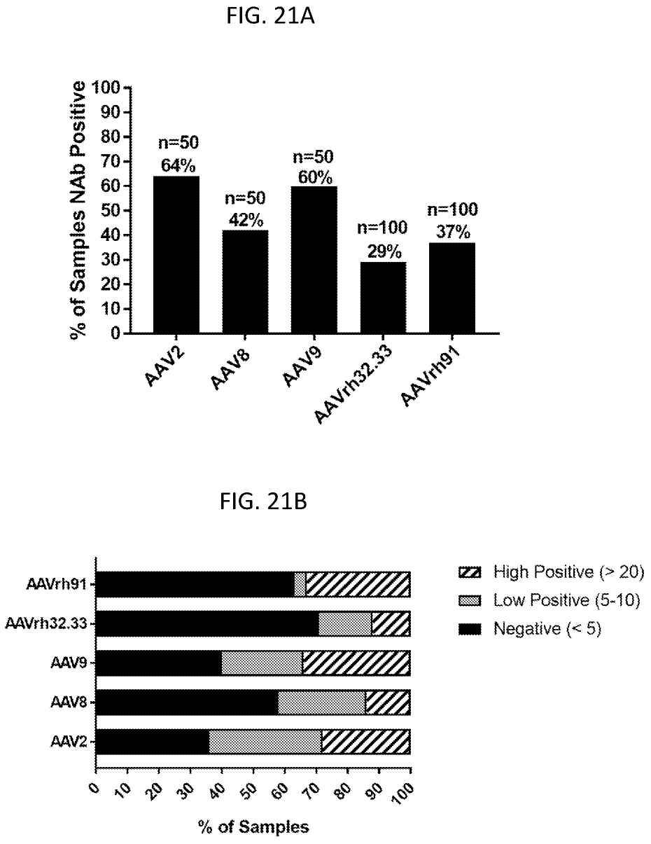

We evaluated the seroprevalence of anti-capsid NAbs against AAVrh91 in the human population using up to 100 random human serum samples (FIG. 21A). We also evaluated NAbs to AAV2, AAV8, AAV9, and AAVrh32.33 in at least 50 of the same samples for comparison. AAVrh91 has a similar seroprevalence (37%) to AAV8 (42%) in the human samples evaluated here, which was reduced compared to that of AAV9 (60%). When we investigated the magnitude of the NAb response, very few samples that were positive for AAVrh91 were in the low positive range (NAb titer of 1/5-1/10). In comparison, the spread of the magnitude of the NAb responses to the other capsids were more spread out, with increased samples reporting in the low positive range (FIG. 21B).

### Example 6: Biodistribution of AAVrh91 Following Systemic Administration

We sought to characterize the biological properties of the AAVrh91 capsid as an AAV vector following systemic administration into animal models. Variable tissue transduction properties were observed in vivo in both mice and rhesus macaques following IV delivery.

### Biodistribution of AAVrh91 in Mice Following Systemic Administration Compared to AAV1, AAV8, and AAV9

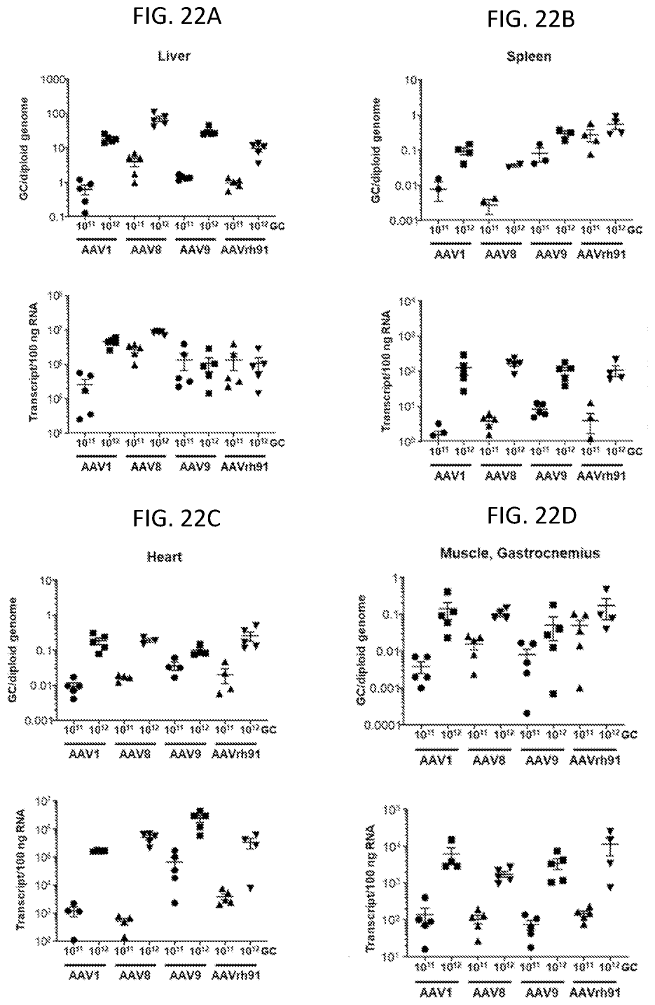

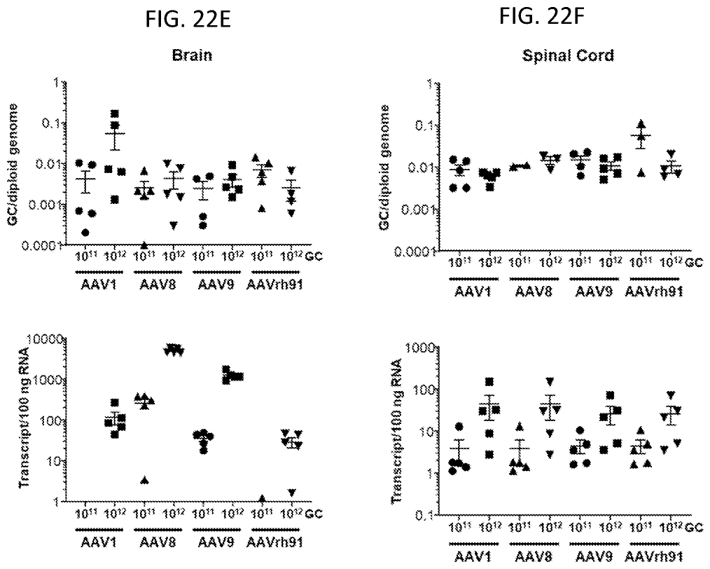

To evaluate the biodistribution and transduction profile of AAVrh91 in a small animal model, we IV administered C57BL/6J mice with 10¹¹ or 10¹² GC of vector expressing eGFP from the CB7 promoter. Mice were also administered the same doses of AAV1, AAV8, and AAV9 vectors. Mice were necropsied at 21 days post-vector administration and liver, heart, and skeletal muscle (gastrocnemius) were harvested. Following isolation of DNA and RNA, samples were evaluated for vector genome copies and vector-derived RNA transcript levels, respectively (FIG. 22A-FIG. 22F).

For all tissues evaluated (liver, heart, and skeletal muscle), there was a dose-dependent increase in vector genome copies for all four capsids evaluated. Administration of the AAV8 vector resulted in the highest vector genome copies and transgene expression in liver. Interestingly, there appeared to be a reduced number of AAVrh91 genome copies in the liver at the high dose (10¹² GC/animal) compared to AAV1, suggesting a potential detargeting of this capsid from the liver. There was no dose effect detected in the transgene RNA levels for the AAV9 and AAVrh91 vectors.

In heart and skeletal muscle, we observed higher genome copies with AAVrh91 than for the other vectors evaluated. While AAVrh91 did not express as highly as AAV9 in the heart, it was similar to AAV8. Interestingly, transgene expression was similar for AAV1, AAV9, and AAVrh91 in skeletal muscle. Tissues samples were also harvested at necropsy for evaluation of GFP expression by fluorescence. Transgene protein expression correlated with RNA levels across the liver, heart, and skeletal muscle (gastrocnemius).

### Evaluation of AAVrh91 in Rhesus Macaques Following Systemic Administration

To evaluate the biodistribution and transduction profile of AAVrh91 following systemic administration in a large animal model, we administered three rhesus macaques with 5×10¹³ GC/kg of AAVrh91.CB7.eGFP. An additional three rhesus macaques were administered with the same dose of AAV9 to directly compare AAVrh91 to the systemic biodistribution of the current best-in-class vector.

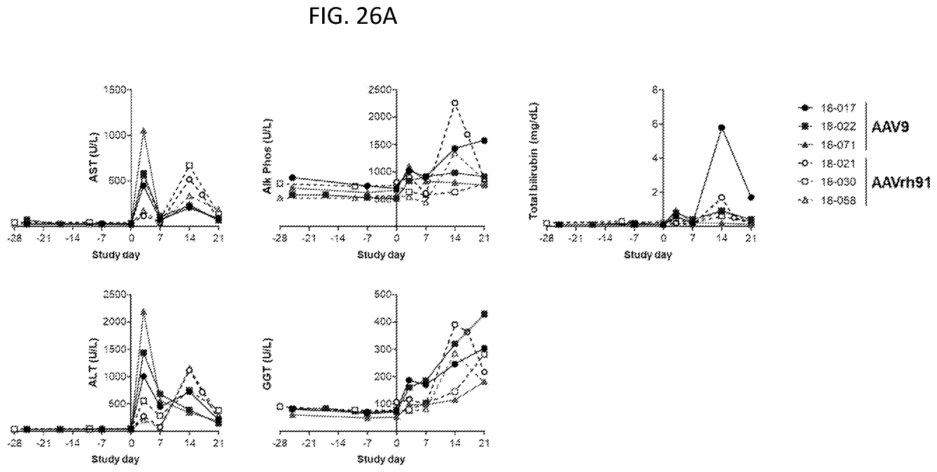

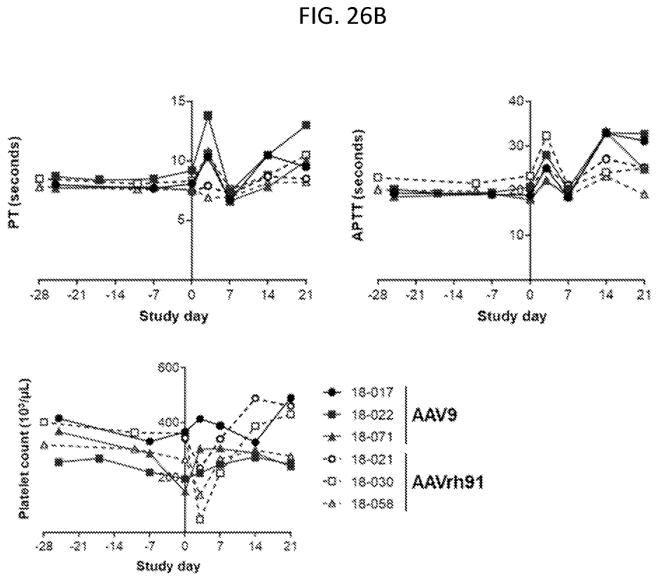

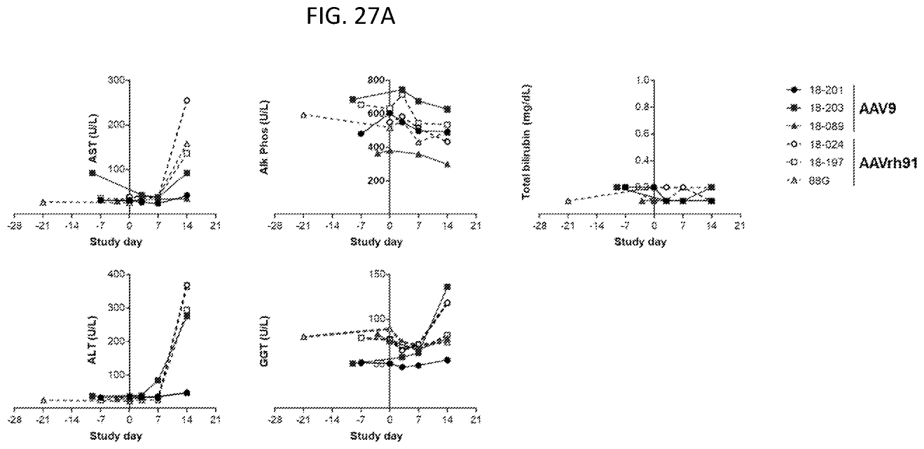

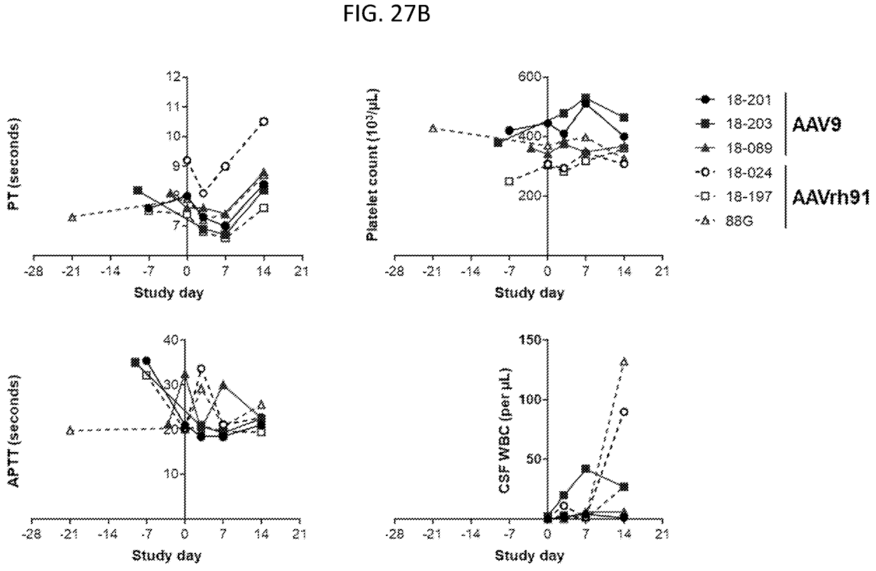

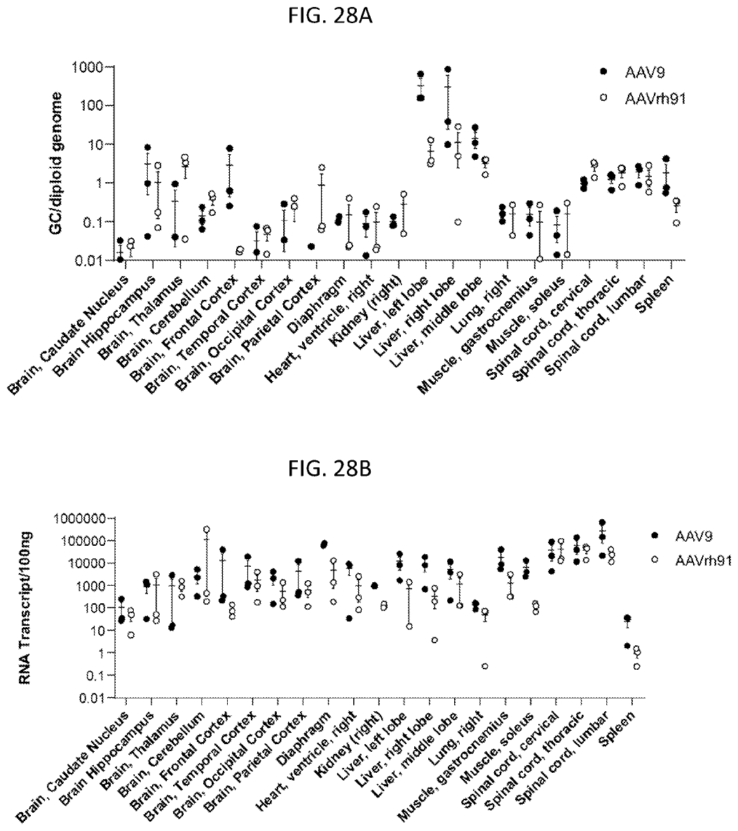

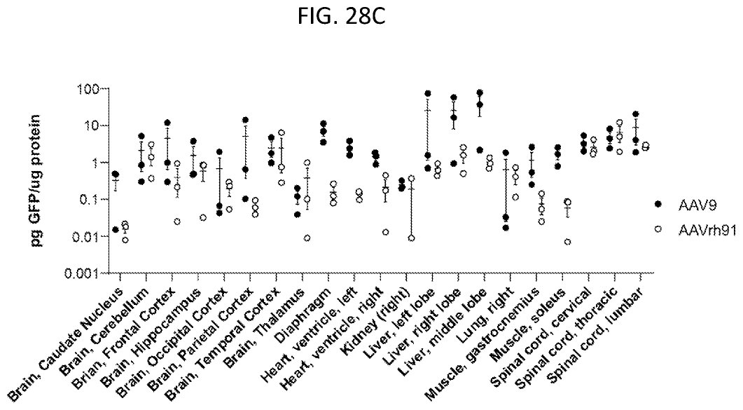

Following IV vector administration, all NHPs were monitored for changes in clinical pathology (FIG. 26A and FIG. 28B). While none of the changes noted reached statistical significance, there were elevations in ALT, AST, and total bilirubin at day 3, which were greater in animals administered AAV9. These elevations quickly reverted to baseline levels by day 7 onwards, with smaller elevations occurring in ALT and AST at day 14 in NHPs administered AAVrh91. Total bilirubin levels also peaked for a second time in many animals at day 14, with one animal that received AAV9 rising to 5.8 mg/dl (18-017). This animal did present with jaundice and was administered subcutaneous fluids but was otherwise stable. This secondary rise in total bilirubin was likely due to the expression of GFP in the liver and the subsequent response to a non-self protein. There were minor elongations in clotting times (PT and APTT) spread across both capsids evaluated on day 3 and some drops in platelet counts were seen in animals administered with AAVrh91.

NHPs were necropsied 21 days post-vector administration and tissues were harvested. To reduce variation due to sampling issues, we evaluated a number of samples from each tissue with the exception of diaphragm, kidney, and spleen, in which only one sample per NHP was evaluated. Both the left and right ventricles were evaluated from heart, samples of three lobes of the liver (left, middle, right), left and right lungs, and 13 skeletal muscles (biceps brachii, biceps femoris, deltoid, extensor carpi radialis, gastrocnemius, gluteus maximus, intercostal, pectoralis major, rectus abdominis, soleus, tibialis anterior, trapezius, and vastus lateralis) were evaluated from the three NHPs per capsid.

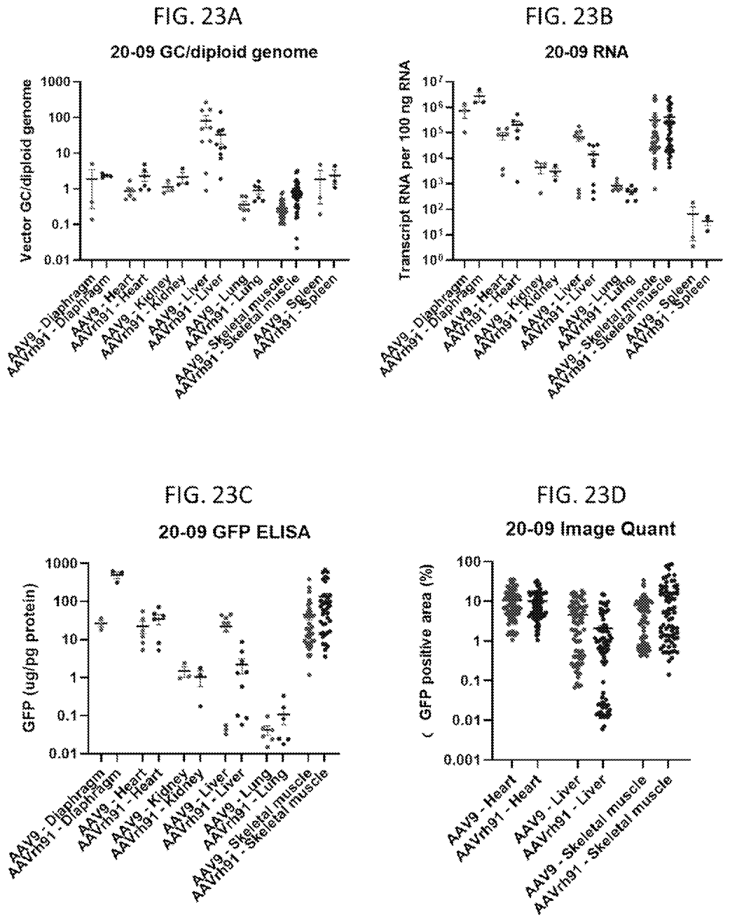

AAV9 and AAVrh91 appeared to have fairly similar vector biodistribution profiles following systemic injection, with the most vector genomes detected in the liver (FIG. 23A). While the difference between the capsids was not statistically significant, NHPs administered AAV9 had 2.5-fold higher vector genome copies in the liver (average of 81.6 GC/diploid genome vs. 32.6 GC/diploid genome for NHPs administered with AAVrh91). While the genome copies in other peripheral organs (heart, kidney, lung, skeletal muscle, and spleen) were up to two logs lower than those in liver, values for AAVrh91 were slightly higher than for AAV9.

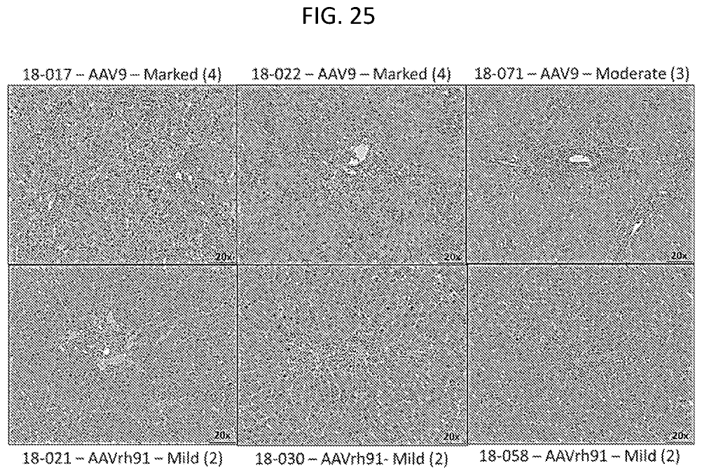

To further evaluate where the transgene was being expressed following intravenous administration, we evaluated transgene RNA copies and GFP protein expression (FIG. 23B, FIG. 23C, and FIG. 23D). While AAV9 had higher transgene RNA levels in kidney, liver, lung, and spleen, AAVrh91 exceeded RNA levels with AAV9 in diaphragm, heart, and skeletal muscle (FIG. 23B). These trends were retained when GFP protein expression was evaluated either by ELISA (FIG. 23C) or by image quantification of GFP expression as detected by IHC (FIG. 23D). Animal 18-017, which received AAV9 vector and had pronounced elevations in serum total bilirubin levels at day 14, had consistently low vector genome copies, transgene RNA levels, and near-absent GFP protein expression in the liver. This is indicative of an immune response to a non-self transgene resulting in depleted transgene expression and clearance of transduced hepatocytes. Histopathology revealed that the most severe liver toxicity (hepatocellular degeneration and individual cell necrosis) was observed in animals 18-017 and 18-022 (2/3, AAV9). When comparing across groups, the severity of liver toxicity was increased in animals that received AAV9 vector (moderate to marked) relative to animals that received AAVrh91 vector (minimal to mild) (FIG. 25).

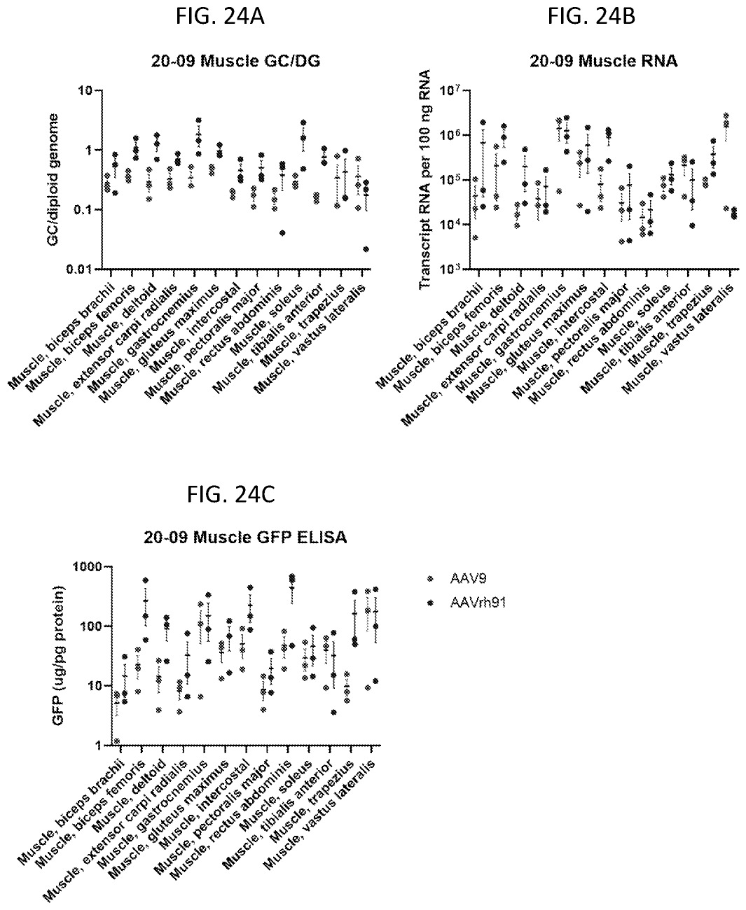

Further analysis of the vector genome copies, transgene RNA levels, and GFP expression in each of the 13 skeletal muscle samples harvested per NHP showed the consistency of AAVrh91 gene transfer and transgene expression. While vector genome copies across the skeletal muscle groups evaluated were consistently increased 0.5-4.6-fold following administration of AAVrh91 compared to AAV9, the differences in the combined data did not reach statistical significance (FIG. 24A). The increased variability in transgene RNA (FIG. 24B) and GFP expression (FIG. 24C) also did not enable the trend towards enhanced transgene expression with AAVrh91 to reach statistically significance.

These studies in both small and large animal models provide a comprehensive analysis of a novel AAV capsid, AAVrh91, which was isolated from a natural source and evaluated as a gene transfer vector. In both mice and rhesus macaques, AAVrh91 has similar, if not increased, transduction of skeletal muscle compared to AAV9. We also observed that AAVrh91 vector expressed less transgene in the liver than the other AAV capsids evaluated. This potential detargeting of the liver by the AAVrh91 capsid could suggest that AAVrh91 vectors have an advantage over capsids in terms of liver toxicity following systemic injection, resulting for less expression of the transgene in liver.

### Example 7: Biodistribution of AAVrh91 Following ICM Administration to Non-Human Primates

Additional studies were performed to evaluate transgene delivery with AAVrh91 capsid following intra-cistern magna (ICM) administration to rhesus macaques.

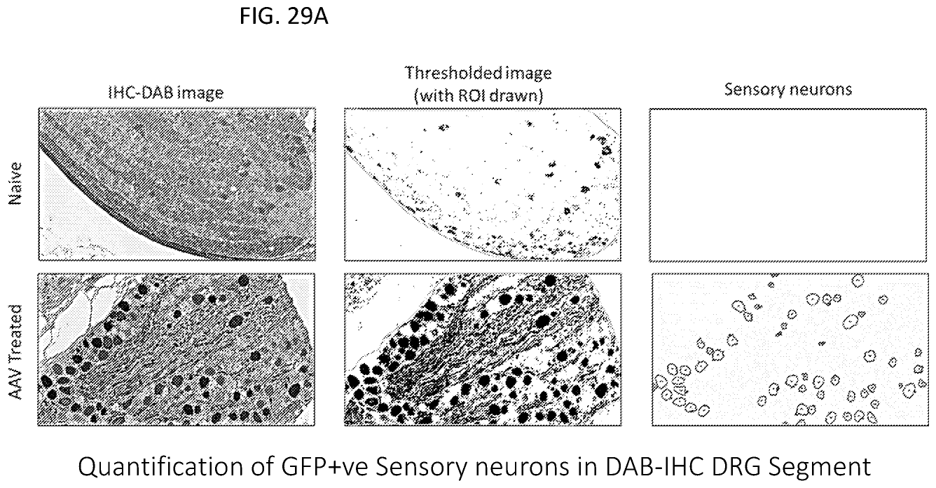

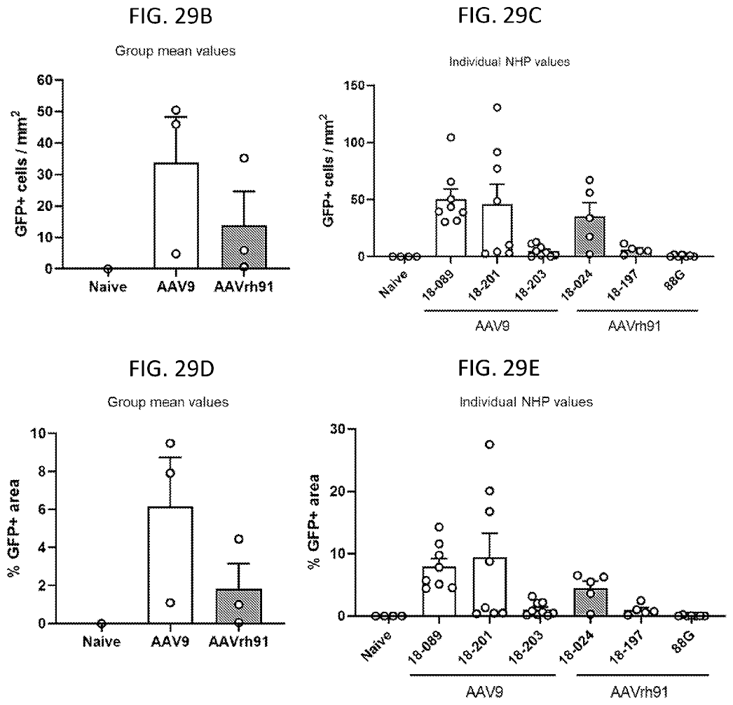

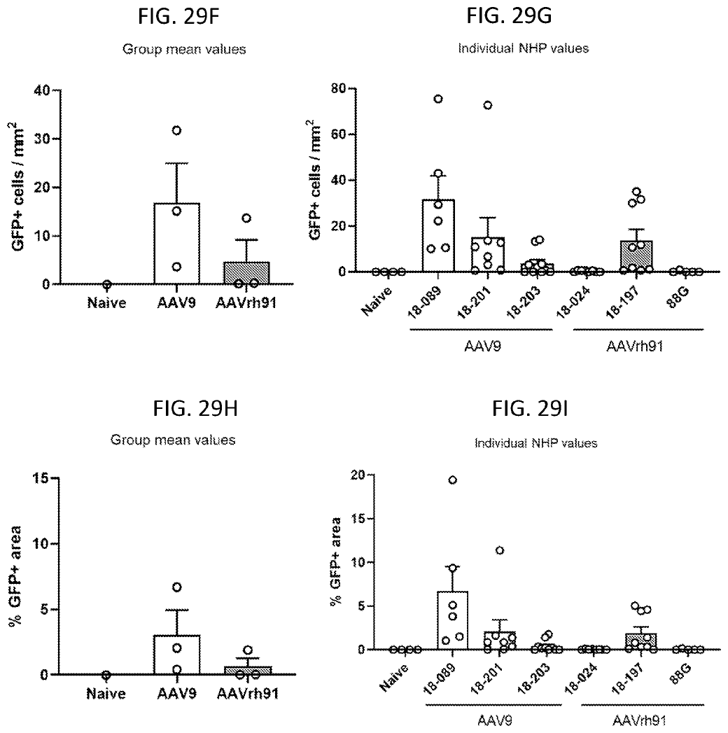

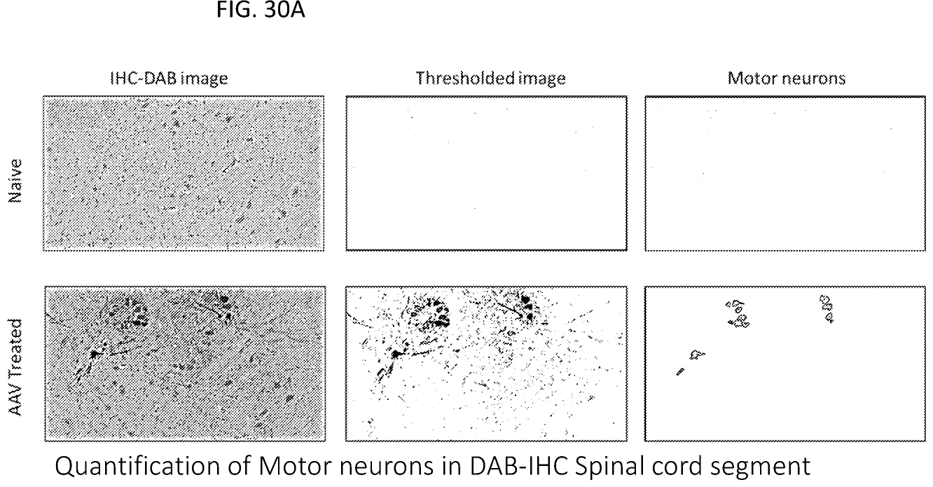

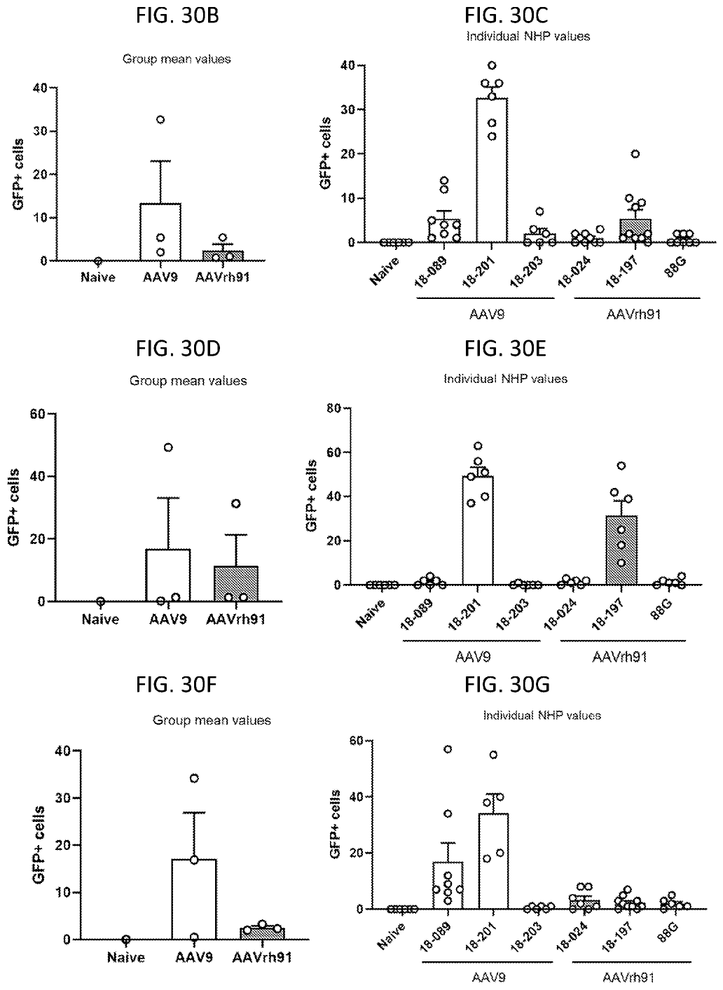

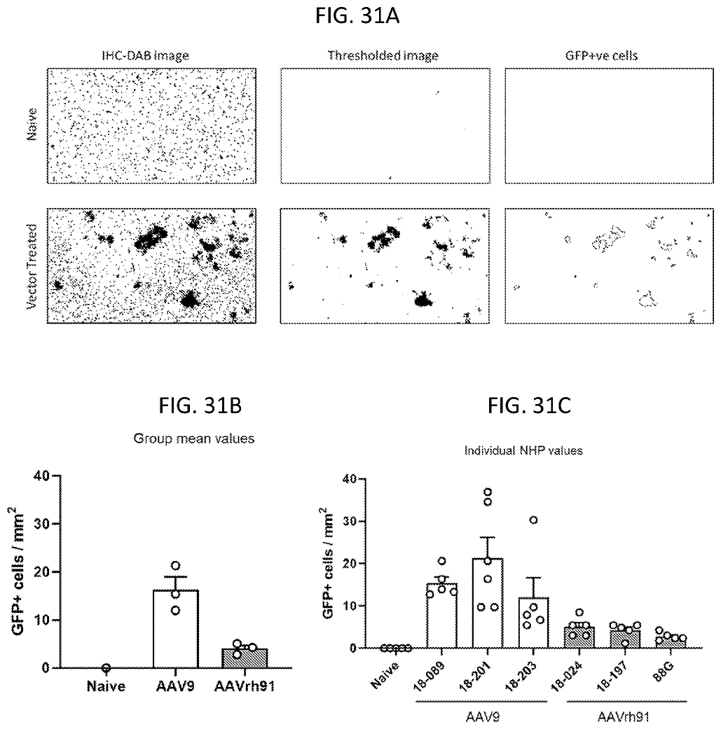

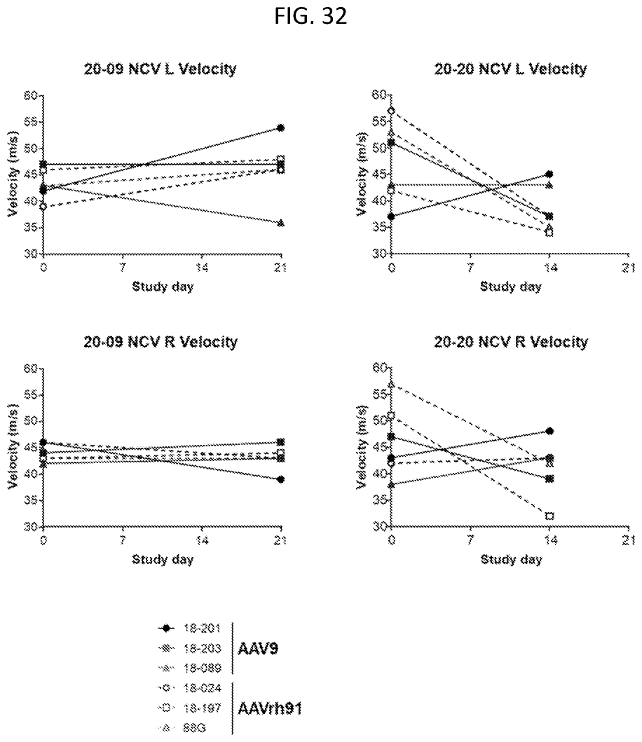

In a first study, 3×10¹³ GC/kg of vector carrying an eGFP transgene, AAVrh91.CB7.eGFP or AAV9.CB7.eGFP, was delivered to NHPs (n=3/group). Necropsy was conducted on day 14 post-administration. Nerve conduction velocity evaluations were performed at baseline and prior to necropsy on day 14 (FIG. 32). Comparisons of biodistribution and transduction profiles are shown in FIG. 28A-FIG. 28C. Immunohistochemistry was performed to quantify GFP positive neurons in brain (FIG. 31A-FIG. 31C), spinal cord (FIG. 30A-FIG. 30G), and dorsal root ganglia (DRG) (FIG. 29A-FIG. 29I). Less transgene expression was observed in DRG in AAVrh91 administered NHPs (compared to AAV9) (FIG. 29A-FIG. 29I). These findings suggest that AAVrh91 gene delivery via the ICM route is likely to be associated with less DRG toxicity than AAV9.

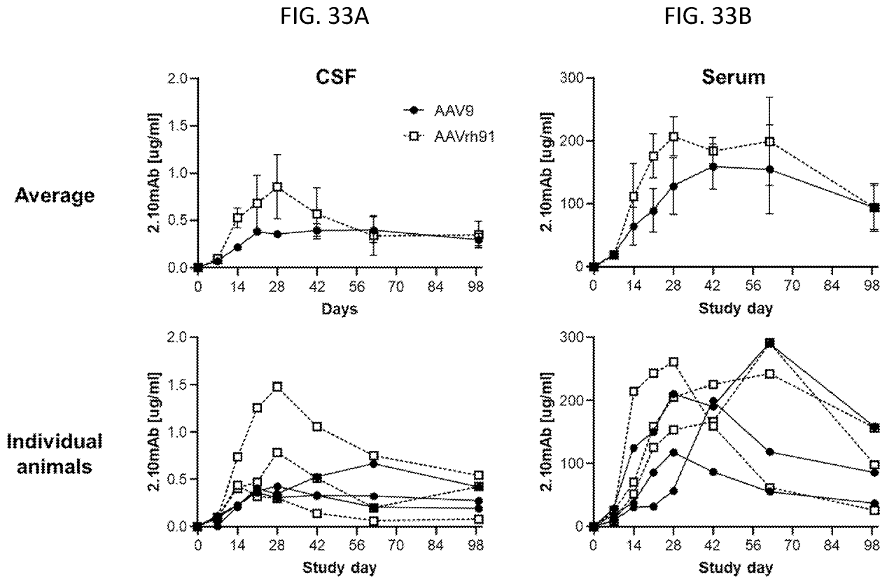

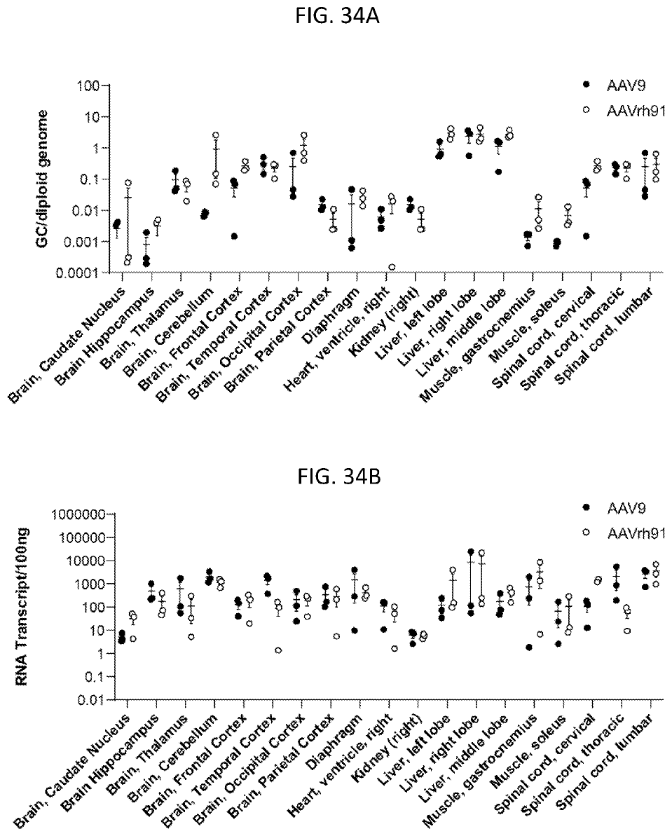

In a further study, 3×10¹³ GC/kg of vector carrying an antibody transgene (2.10A mAb), AAVrh91.CB7. 2.10A or AAV9.CB7.2.10A, was delivered to NHPs (n=3/group). Serum and CSF were monitored for 2.10A mAb expression (FIG. 33A and FIG. 33B). Necropsy was performed at day 90 post vector administration and tissues were collected for analysis of vector biodistribution (FIG. 34A and FIG. 34B).

### Example 8: Cryo-EM Structural Data Comparing AAVrh91 and AAV1 Capsids

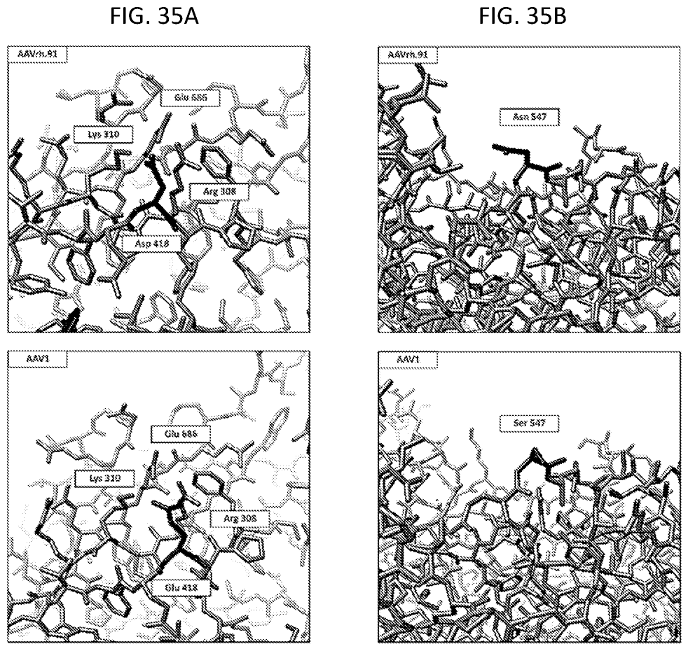

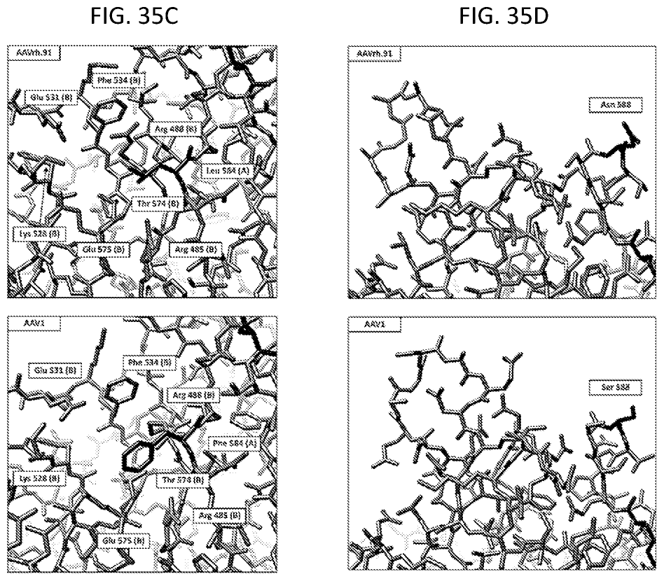

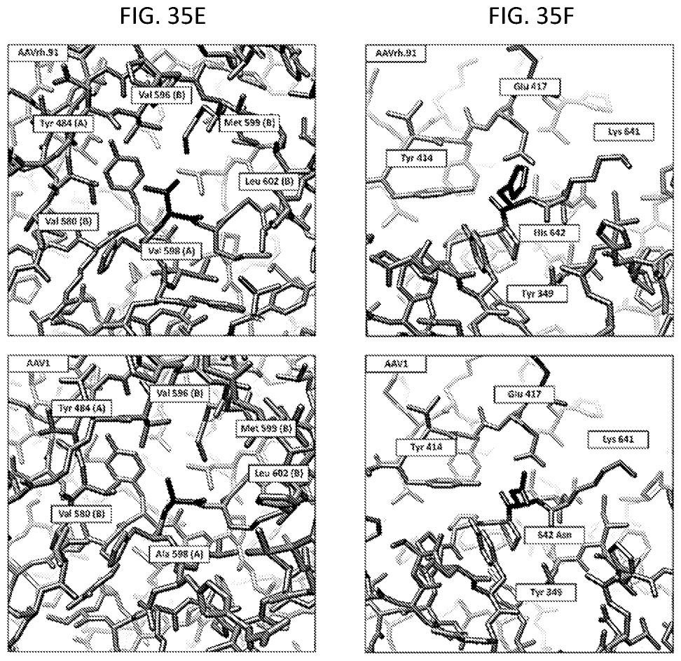

To provide mechanistic insights into the improved properties of the AAVrh91 vector, we used cryo-electron microscopy to solve a structure for this vector at 2.33 Å resolution. We compared our structure to a previously published structure for AAV1, the most widely used Clade A vector in clinical trials. AAVrh91 differs from AAV1 at 11 amino acid positions, 6 of which are located in the capsid's VP3 protein and are surface exposed. See FIG. 35A-FIG. 35F.

### Results

Asp 418 in AAVrh91 and Glu 418 in AAV1 are solvent-exposed residues located on the interior surface of the AAV capsid, in close proximity to other charged residues Arg 308, Lys 310, and Glu 686 (FIG. 35A). Asp and Glu are both acidic residues that carry a negative charge at neutral pH, and can be seen to adopt similar confirmations in both structures. Structural changes to the charged residues surrounding position 418 are not observed. Overall, the change from Glu 418 to Asp 418 is very conservative, and would likely have a negligible impact on capsid function.

Asn 547 in AAVrh91 and Ser 547 in AAV1 are solvent-exposed residues located in HVR VII on the exterior surface of the AAV capsid, and are not in close proximity to other amino acids (FIG. 35B). Both are polar amino acids that are uncharged at neutral pH, but possess different functional groups. Asn contains a carbonyl and amine functional group on its side chain, and Ser contains a single hydroxyl group. Their solvent exposure on the exterior of the capsid means that these residues are available to interact with cellular receptors, though to date no AAV structure-function relationships have been defined for residues at this position. Overall, this change is also conservative, but the differences in functional groups between the amino acids have the potential to impact capsid function.

Leu 584 in AAVrh91 and Phe 584 in AAV1 are solvent-exposed residues located in HVR VIII on the exterior surface of the AAV capsid, in close proximity to Arg 485, Arg 488, Lys 528, Glu 531, Phe 534, Thr 574, and Glu575, which are all located on an adjacent chain (FIG. 35C). Leu is a small hydrophobic amino acid, and Phe is a large hydrophobic amino acid. The residues in close proximity to position 584 are all charged amino acids, with the exception of Phe 534 (hydrophobic) and Thr 574 (polar). In AAVrh91 the smaller Leu residue may be less disruptive to these proximal charged residues than the larger Phe in AAV1. Lessened disruption to this charged pocket could function to increase capsid stability. Given the prevalence of interchain contacts at this position, the change from Phe to Leu at position 584 may partially explain the increased manufacturing yields observed for AAVrh91 relative to AAV1.

Asn 588 in AAVrh91 and Ser 588 in AAV1 are solvent-exposed residues located in HVR VIII on the exterior surface of the AAV capsid, at the tip of the AAV's 3-fold spike structure, and are not in close proximity to other amino acids (FIG. 35D). As mentioned above, both residues are polar amino acids that are uncharged at neutral pH, but possess different functional groups that could impact capsid/receptor interactions. Position 588 is significant in that it is a common location used for peptide insertions in protein engineering efforts to alter AAV tropism. This is because its prominent location and high level of solvent exposure increases potential for interacting with cellular receptors. Due to its enhanced exposure, this Ser to Asn mutation is more likely to influence capsid function than the same mutation observed at position 547.

Val 598 in AAVrh91 and Ala 598 in AAV1 are also located in HVR VIII, but are not in a highly solvent exposed location. Instead, these small hydrophobic residues participate in the formation of a hydrophobic pocket with adjacent residues Tyr 484, Val 580, Val 596, Met 599, and Leu 602 (FIG. 35E). These residues are located in the center of the AAV's 3-fold axis where three VP3 proteins come together, and have a number of contacts with adjacent peptide chains. Ala, found at position 598 in AAV1, is the smallest hydrophobic amino acid, whereas Val 598 in AAVrh91 is slightly larger and more hydrophobic. The Val residue appears to fill the space within this hydrophobic pocket better than its smaller Ala counterpart, which may improve capsid stability. Given the central location of this hydrophobic pocket and its number of inter-chain contacts, this Ala/Val substitution at position 598 is the most likely explanation for the manufacturing advantages observed with AAVrh91.

His 642 in AAVrh91 and Asn 642 in AAV1 are solvent-exposed residues located on the interior surface of the AAV capsid, in proximity to polar residues Tyr349 and Tyr414, and charged residues Glu417 and Lys641 (FIG. 35F). His is a basic residue that carries a positive charge at neutral pH, and Asn is a polar residue that is uncharged at neutral pH. The Asn/His substitution at position 642 does not induce any observable structural changes in the surrounding hydrophilic residues. Overall, the change from Asn 642 to His 642 results in an increase in local positive charge, but its location on the interior of the capsid and minimal impact on surrounding capsid structure suggests that this change would not dramatically alter capsid function.

### Example 9: AAVrh91 Vector Production Optimization

Multiple strategies were utilized to alter trans production plasmids for AAVrh91 to improve AAVrh91 vector yields.

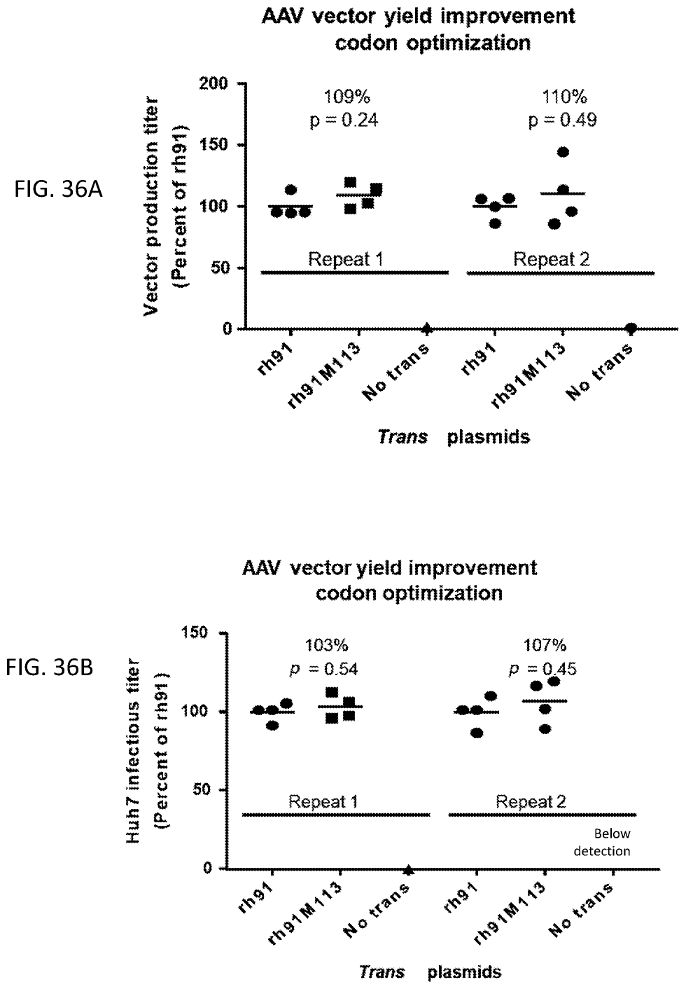

One strategy was engineering the AAVrh91 capsid gene sequence, including optimizing codon usage. The sequence generated (rh91M113, AAVrh91eng, SEQ ID NO: 3) differs from the native AAVrh91 coding sequence at 113 nucleotides but encodes the same amino acid sequence. For each version, we re-transformed the plasmid and randomly picked four clones for individual triple-transfections in 12-well plates. Vector yields were determined by two methods: qPCR for production titers (FIG. 36A), and Huh7 transduction for infectious titers (FIG. 36B). We observed that rh91M113 had improved yields in repeat experiments using both measurements, although the differences were not statistically significant (none of the p values were less than 0.05).

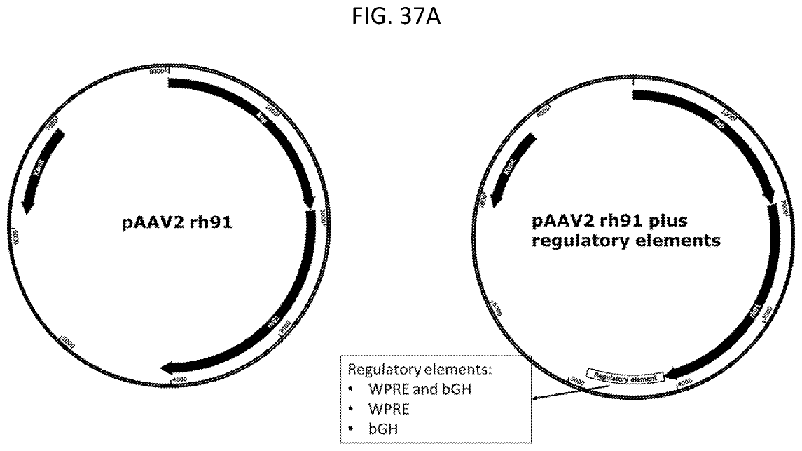

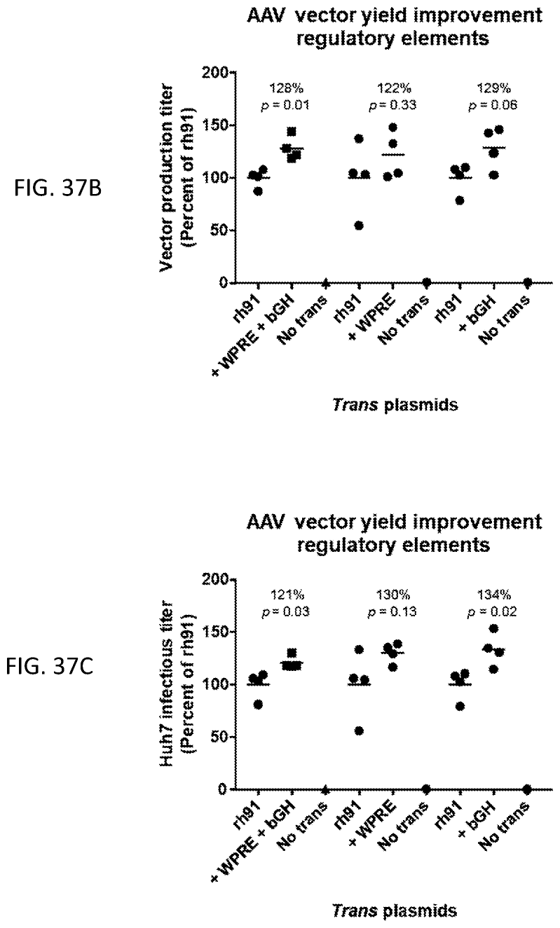

A second strategy was adding regulatory elements to the trans plasmids. We generated plasmids that included either or both of a woodchuck hepatitis virus post-transcriptional regulatory element (WPRE) and a bovine growth hormone polyadenylation (bGH polyA) signal (FIG. 37A). Vector yields were evaluated using the methods described above. The results indicated that the inclusion of the regulator elements (WPRE and bGH polyA, WPRE alone, and bGH polyA alone) can improve vector yields (FIG. 37B and FIG. 37C).

### Sequence Listing Free Text

The following information is provided for sequences containing free text under numeric identifier <223>.

| SEQ ID NO: | Free Text under <223> |
|---|---|
| 3 | <223> synthetic construct |
| | <220> |
| | <221> CDS |
| | <222> (1) . . . (2211) |
| 4 | <223> Synthetic Construct |
| 5 | <223> AAV6 mutant |
| | <220> |
| | <221> CDS |
| | <222> (1) . . . (2211) |
| 6 | <223> Synthetic Construct |
| 9 | <223> primer sequence |
| 10 | <223> primer sequence |
| 11 | <223> primer sequence |
| 12 | <223> primer sequence |
| 13 | <223> miRNA target sequence |
| 14 | <223> miRNA target sequence |

All documents cited in this specification are incorporated herein by reference. U.S. Provisional Patent Application No. 62/840,1840, filed Apr. 29, 2019, U.S. Provisional Patent Application No. 62/913,314, filed Oct. 10, 2019, U.S. Provisional Patent Application No. 62/924,095, filed Oct. 21, 2019, U.S. Provisional Patent Application No. 63/065,616, filed Aug. 14, 2020, U.S. Provisional Patent Application No. 63/109,734, filed Nov. 4, 2020, and International Patent Application No. PCT/US2020/030266, filed Apr. 20, 2020, are incorporated by reference in their entireties, together with their sequence listings. The sequence listing filed herewith labeled “21-9545PCT_ST25” and the sequences and text therein are incorporated by reference. While the invention has been described with reference to particular embodiments, it will be appreciated that modifications can be made without departing from the spirit of the invention. Such modifications are intended to fall within the scope of the appended claims.

1. A method of delivering of a transgene to one or more target cells of the central nervous system (CNS) of a subject, the method comprising administering to the subject a recombinant adeno-associated virus (AAV) vector comprising an AAVrh91 capsid and a vector genome comprising the transgene operably linked to regulatory sequences that direct expression of the transgene in the target cells of the CNS.

2. The method according to claim 1, wherein the target cells of the CNS are parenchymal cells, cells of the choroid plexus, ependymal cells, astrocytes, and/or and neurons, optionally neurons of the cortex, hippocampus, and/or striatum.

3. The method according to claim 1, wherein the transgene encodes a secreted gene product.

4. The method according to claim 1, wherein the AAV vector is delivered intrathecally, optionally via intra-cisterna magna (ICM) injection.

5. The method according to claim 1, wherein the AAV vector is delivered via intraparenchymal administration.

6. (canceled)

7. A method for detargeting the liver and/or reducing liver toxicity following systemic administration of an AAV vector to a subject, the method comprising administering to the subject via intravenous injection a recombinant AAV vector comprising an AAVrh91 capsid and a vector genome comprising a transgene operably linked to regulatory sequences that direct expression of the transgene in cells of the liver, wherein levels of transduction of the liver and/or liver toxicity observed following administration of the AAV vector are reduced relative to an AAV vector having an AAV1, AAV8, and/or AAV9 capsid.

8. The method according to claim 7, wherein the AAVrh91 capsid comprises a capsid protein comprising the amino acid sequence of SEQ ID NO: 2.

9. The method according to claim 7, wherein the AAVrh91 capsid comprises a capsid protein produced by expression of a nucleotide sequence of SEQ ID NO: 1 or 3, or a sequence sharing at least 90%, at least 95%, at least 97%, at least 98% or at least 99% identity a nucleotide sequence of SEQ ID NO: 1 or 3.

10. The method according to claim 7, wherein the AAVrh91 capsid comprises a capsid protein wherein the capsid protein is encoded by a nucleotide sequence of SEQ ID NO: 1 or 3.

11. The method according to claim 1, wherein the AAVrh91 capsid comprises capsid proteins comprising:
(1) a heterogeneous population of AAVrh91 vp1 proteins selected from: vp1 proteins produced by expression from a nucleic acid sequence which encodes the predicted amino acid sequence of 1 to 736 of SEQ ID NO: 2, vp1 proteins produced from SEQ ID NO: 1 or 3, or vp1 proteins produced from a nucleic acid sequence at least 70% identical to SEQ ID NO: 1 or 3 which encodes the predicted amino acid sequence of 1 to 736 of SEQ ID NO: 2,
a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 2, vp2 proteins produced from a sequence comprising at least nucleotides 412 to 2208 of SEQ ID NO: 1 or 3, or vp2 proteins produced from a nucleic acid sequence at least 70% identical to at least nucleotides 412 to 2208 of SEQ ID NO: 1 or 3 which encodes the predicted amino acid sequence of at least about amino acids 138 to 736 of SEQ ID NO: 2,
a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 2, vp3 proteins produced from a sequence comprising at least nucleotides 607 to 2208 of SEQ ID NO: 1 or 3, or vp3 proteins produced from a nucleic acid sequence at least 70% identical to at least nucleotides 607 to 2208 of SEQ ID NO: 1 or 3 which encodes the predicted amino acid sequence of at least about amino acids 203 to 736 of SEQ ID NO: 2; and/or
(2) a heterogeneous population of vp1 proteins which are the product of a nucleic acid sequence encoding the amino acid sequence of SEQ ID NO: 2, a heterogeneous population of vp2 proteins which are the product of a nucleic acid sequence encoding the amino acid sequence of at least about amino acids 138 to 736 of SEQ ID NO: 2, and a heterogeneous population of vp3 proteins which are the product of a nucleic acid sequence encoding at least amino acids 203 to 736 of SEQ ID NO: 2, wherein: the vp1, vp2 and vp3 proteins contain subpopulations with amino acid modifications comprising at least two highly deamidated asparagines (N) in asparagine-glycine pairs in SEQ ID NO: 2 and optionally further comprising subpopulations comprising other deamidated amino acids, wherein the deamidation results in an amino acid change.

12. The method according to claim 11, wherein the nucleic acid sequence encoding the capsid proteins is SEQ ID NO: 1 or 3, or a sequence at least 80% to at least 99% identical to SEQ ID NO: 1 or 3 which encodes the amino acid sequence of SEQ ID NO: 2.

13. The method according to claim 11, wherein the nucleic acid sequence is at least 80% identical to SEQ ID NO: 1 or 3.

14-20. (canceled)

21. A method of generating a recombinant AAV comprising the steps of culturing a host cell containing: (a) a nucleic acid molecule encoding an AAV capsid protein having an amino acid substitution at one or more of position 418, 547, 584, 588, 598, and 642 (when aligned with SEQ ID NO: 2); (b) a functional rep gene; (c) a minigene comprising an AAV 5′ ITR, an AAV 3′ ITR, and a transgene; and (d) sufficient helper functions to permit packaging of the minigene into an AAV capsid.

22. The method according to claim 21, wherein the generated recombinant AAV has improved production yields and/or altered cell or tissue tropism relative to an unmodified capsid protein.

23. The method according to claim 21, wherein the generated recombinant AAV transduces cells of the CNS at higher levels relative to an unmodified capsid protein.

24. The method according to claim 21, wherein the nucleotide sequence of (a) encodes an clade A capsid protein having a substitution at one or more of the recited positions.

25. The method according to claim 21, wherein the nucleotide sequence of (a) encodes an AAV1, AAVhu48R3, AAVhu48, AAVhu44, AAV.VR-355, AAV.VR-195, AAV6, or AAV6.2 capsid having one or more of the recited substitutions.

26. The method according to claim 21, wherein the nucleotide sequence of (a) encodes the amino acid sequence of a capsid protein having one or more amino substitutions selected from: Asp at position 418, Asn at position 547, Leu at position 584, Asn at position at 588, Val at position 598, and His at position 642.

27. The method according to claim 21, wherein the nucleotide sequence of (a) encodes the amino acid sequence of SEQ ID NO: 8 (AAV1) having amino acid substitutions at Glu418, Ser547, Phe584, Ser588, Ala598, and/or Asn642, and wherein the encoded amino acid sequence is at least 95% identical or at least 99% identical to SEQ IN NO: 8.

28. The method according to claim 21, wherein the nucleotide sequence of (a) encodes the amino acid sequence of SEQ ID NO: 8 (AAV1) having one or more amino substitutions selected from: Asp at position 418, Asn at position 547, Leu at position 584, Asn at position at 588, Val at position 598, and His at position 642, and wherein the encoded amino acid sequence is at least 95% identical or at least 99% identical to SEQ IN NO: 8.

29-41. (canceled)
